(19) United States
(12) Patent Application Publication
(10) Pub. No.: US 2010/0099737 A1
(43) Pub. Date: Apr. 22, 2010

(54) COMPOSITIONS AND METHODS FOR TREATING MYELOSUPPRESSION

(76) Inventors: Gerald Krystal, Vancouver (CA); Christopher Ong, Vancouver (CA); Alice Mui, Vancouver (CA)

Correspondence Address: CLARK & ELBING LLP, 101 FEDERAL STREET, BOSTON, MA, 02110, US

(21) Appl. No.: 12/438,471

(22) PCT Filed: Aug. 24, 2007
(86) PCT No.: PCT/CA07/01501

§ 371 Date: Nov. 13, 2009

Related U.S. Application Data
(60) Provisional application No. 60/823,404, filed on Aug. 24, 2006.

(51) Int. Cl. A61K 31/7105; G01N 33/53; A61P 35/00

(52) U.S. Cl. 514/44 A; 435/7.21

(57) ABSTRACT

The invention provides, in part, compositions and methods for protecting a hemopoietic cell, or for treating myelosuppression, in a subject in need thereof, by administering an effective amount of an inhibitor of a SH2-containing inositol-5′-phosphatase.

### CROSS-REFERENCE TO RELATED APPLICATIONS

This application claims the benefit of U.S. provisional application No. 60/823,404, filed Aug. 24, 2006, which is hereby incorporated by reference.

### FIELD OF INVENTION

The invention provides compositions and methods for protection against and treatment of myelosuppression. More specifically, the invention provides inhibitors of SH2-containing inositol-5′-phosphatase for protection against hemodepletion and treatment of myelosuppression.

### BACKGROUND OF THE INVENTION

The phosphatidylinositol (PI) 3-kinase (PI3K) pathway plays a central role in regulating many biological processes, including survival and proliferation, through the generation of the potent second messenger, PIP3. This phospholipid is present at low levels in the plasma membrane of unstimulated cells but is rapidly synthesized from PI-4,5-P2 by PI3K in response to a diverse array of extracellular stimuli (reviewed in 11). This transiently generated PIP3 attracts pleckstrin homology (PH) domain-containing proteins, such as the survival/proliferation enhancing serine/threonine kinase Akt (also known as protein kinase B (PKB)), to the plasma membrane to mediate its effects (reviewed in 1,12). Activation of the PI3K/Akt pathway has been linked with resistance to chemotherapeutic drugs and to radiation₁₃, and its down regulation via PI3K inhibitors lowers the resistance of tumour cell lines to various types of therapy^{14,15}. To ensure that activation of the PI3K pathway is appropriately suppressed/terminated, the ubiquitously expressed tumour suppressor PTEN hydrolyzes PIP3 to PI-4,5-P2 while the hemopoietic restricted SH2-containing inositol-5′-phosphatase 1 (SHIP1), stem cell SHIP (sSHIP) (which is transcribed from a promoter between exons 5 and 6 of the SHIP gene and is expressed in embryonic stem (ES) cells¹⁶ and co-expressed, albeit at low levels, with SHIP1 in HSCs¹⁶), and the more widely expressed SHIP2 break it down to PI-3,4-P2. Within non-hemopoietic cells, PTEN and SHIP2 appear to be the key enzymes that keep PIP3 levels suppressed while in hemopoietic cells, SHIP1 is the central player.

SHIP1 (also known as SHIP), has been implicated as a negative regulator of proliferation/survival, differentiation and end cell activation in hemopoietic cells by translocating to membranes following extracellular stimulation and hydrolysing the PI3K-generated second messenger, PI-3,4,5-P3 (PIP3) to PI-3,4-P2₁. Myeloid progenitors in SHIP−/− mice display enhanced survival and proliferation and this results in an increased number of mature neutrophils and monocyte/macrophages².

A major limitation in treating patients with chemotherapies or radiotherapies is the toxicity of these treatments to bone marrow (BM) cells. This leads to myelosuppression which results in anemia, requiring red blood cell transfusions, and increased susceptibility to infections because of a drop in white blood cells (leukocytes) and/or increased bleeding because of insufficient numbers of platelets. This myelosuppression limits the chemotherapy or radiation doses that can be given, for example, to cancer patients which in turn limits the likelihood of tumour eradication. Current strategies to replenish the BM deficit that follows these treatments include BM transplantation (which is costly and exposes patients to potentially lethal graft versus host disease) and the administration of cytokines such as erythropoietin (Epo or Epogen), G-CSF (Neupogen) and GM-CSF) to stimulate hemopoietic progenitor proliferation along various differentiation pathways. However, some patients do not respond to these cytokines and none of these treatments reverse the fall in platelet numbers. Additionally, the cost of administering even single cytokines is so prohibitive that most BM transplant facilities do not currently use them to narrow the “septic window” following these transplants and these patients are thus at high risk of dying from trivial infections.

### SUMMARY OF THE INVENTION

The invention provides, in part, compositions and methods for protecting a hemopoietic cell, or for treating myelosuppression, in a subject in need thereof, by administering an effective amount of an inhibitor of a SH2-containing inositol-5′-phosphatase.

In one aspect, the invention provides a method of protecting a hemopoietic cell in a subject in need thereof by administering an effective amount of an inhibitor of a hemopoietic-restricted SH2-containing inositol-5′-phosphatase to the subject.

In alternative embodiments, the hemopoietic cell may be a hemopoietic progenitor cell, such as a myeloid progenitor cell or a lymphoid progenitor cell, or may be a mature cell. In alternative embodiments, the protecting includes decreasing cell death (e.g., apoptosis). In alternative embodiments, the cell death may be induced by chemotherapy or by radiotherapy. In alternative embodiments the hemopoietic-restricted SH2-containing inositol-5′-phosphatase may be a SHIP1 molecule. In alternative embodiments, the subject may be a human. In alternative embodiments, the subject may have, or may be suspected of having, a cancer (e.g., a solid tumor). In alternative embodiments, the subject may be undergoing chemotherapy or radiotherapy. In alternative embodiments, the chemotherapy may be a cancer therapy (e.g., cisplatin, doxorubicin, or taxotere). In alternative embodiments, the method further comprises administering a chemotherapeutic agent (e.g., a cancer therapeutic agent, such as cisplatin, doxorubicin, or taxotere) or administering a radiotherapy. The inhibitor may be administered before, during or after administration of said chemotherapeutic agent or said radiotherapy. The inhibitor may be a siRNA, e.g., a sequence consisting essentially of AAGAGTCAGGAAGGAGAGAAT (SEQ ID NO: 10) or AAGAGTCAGGAAGGAGAAAAT (SEQ ID NO: 11), or a small molecule.

In alternative aspects, the invention provides a method of treating myelosuppression (e.g., immune suppression) in a subject in need thereof by administering an effective amount of an inhibitor of a hemopoietic-restricted SH2-containing inositol-5′-phosphatase to the subject.

In alternative embodiments, the myelosuppression includes a decrease in hemopoietic progenitor cells or mature cells. In alternative embodiments, the treating includes increasing proliferation of a hemopoietic cell or includes reducing death of a hemopoietic cell. In alternative embodiments, the myelosuppression may be induced by chemotherapy or by radiotherapy. In alternative embodiments, the hemopoietic-restricted SH2-containing inositol-5′-phosphatase may be a SHIP1 molecule. In alternative embodiments, the subject may have, or may be suspected of having, a cancer e.g., a solid tumor. In alternative embodiments, the subject may be a human. In alternative embodiments, the subject may be undergoing chemotherapy or radiotherapy. In alternative embodiments, the chemotherapy may be a cancer therapy. In alternative embodiments, the cancer therapy may be one or more of cisplatin, doxorubicin, or taxotere. In alternative embodiments, the inhibitor may be administered after administration of said chemotherapy or said radiotherapy. In alternative embodiments, the inhibitor may be a siRNA or a small molecule. In alternative embodiments, the siRNA may consist essentially of the sequence AAGAGTCAGGAAGGAGAGAAT (SEQ ID NO: 10) or AAGAGTCAGGAAGGAGAAAAT (SEQ ID NO: 11).

In an alternative aspect, the invention provides an siRNA molecule consisting essentially of the sequence AAGAGTCAGGAAGGAGAGAAT (SEQ ID NO: 10) or AAGAGTCAGGAAGGAGAAAAT (SEQ ID NO: 11).

In an alternative aspect, the invention provides a pharmaceutical composition comprising an siRNA molecule as described herein in combination with a pharmaceutically acceptable carrier.

In an alternative aspect, the invention provides a pharmaceutical composition as described herein further comprising a chemotherapeutic agent. The chemotherapeutic agent may be one or more of cisplatin, doxorubicin, or taxotere.

In an alternative aspect, the invention provides a kit comprising an siRNA molecule as described herein, together with instructions for use in treating myelosuppression.

In an alternative aspect, the invention provides a use of an inhibitor of a SH2-containing inositol-5′-phosphatase in the preparation of a medicament for protecting a hemopoietic cell in a subject in need thereof.

In an alternative aspect, the invention provides a use of an inhibitor of a SH2-containing inositol-5′-phosphatase in the preparation of a medicament for treating myelosuppression in a subject in need thereof. In alternative embodiments, the myelosuppression includes immune suppression.

In an alternative aspect, the invention provides a method for screening for an inhibitor of a hemopoietic-restricted SH2-containing inositol-5′-phosphatase, by providing a test compound and a control compound; contacting a hemopoietic cell with the test compound or the control compound; and determining whether the test compound may be capable of increasing the survival or proliferation of the hemopoietic cell compared to the control compound; where a test compound that increases the survival or proliferation of the hemopoietic cell compared to the control compound may be an inhibitor of a SH2-containing inositol-5′-phosphatase.

This summary of the invention does not necessarily describe all features of the invention.

### BRIEF DESCRIPTION OF THE DRAWINGS

These and other features of the invention will become more apparent from the following description in which reference is made to the appended drawings wherein:

FIGS. 1A-H show siRNA-mediated inhibition of SHIP expression. (A-C) Immunoblot analyses of the EL-4 cell line transduced with siRNAs to SHIP, as indicated or a control non-silencing siRNA (NS) and assessed for SHIP and control GAPDH protein expression on the indicated days; (D-E) Immunoblot analyses of the TF1 hemopoietic progenitor cell line transduced with siRNA to SHIP (siSHIP or as indicated) or a control non-silencing siRNA (siNS or NS) and assessed for SHIP and control GAPDH protein expression on the indicated days. (F) Immunoblot analyses of TF1 cells transfected with siSHIP or siNS, stimulated with the cytokine GM-CSF for the indicated length of time, and probed with antibodies against SHIP, the PIP3 dependent kinase PKB or phospho PKB (Ser 473). (G) Graph of TF-1 cells transfected with siSHIP (triangles) or siNS (squares) in the absence of growth factors. (H) Graph of TF-1 cells transfected with siSHIP (open diamonds) and control siNS (solid diamonds), cultured in the presence of increasing concentrations of the growth promoting cytokine interleukin-5 (IL-5), 2 days after siRNA transfection.

FIG. 2 shows a bar graph of TF1 cells transfected with SHIP siRNA or control siRNA and proliferation assessed by ³H-thymidine incorporation at the indicated concentrations of cisplatin, doxorubicin and taxotere.

FIGS. 3A-C show (A-B) the nucleotide (SEQ ID NO: 1) and (C) amino acid (SEQ ID NO: 2) sequence of human SHIP1; GenBank Accession No. U57650.

FIGS. 4A-C show (A-B) the nucleotide (SEQ ID NO: 3) and (C) amino add sequence (SEQ ID NO: 4) of mouse SHIP1; GenBank Accession No. U39203.

### DETAILED DESCRIPTION

The invention provides, in part, compositions and methods for down-modulating SH2-containing inositol-5′-phosphatase (SHIP) to protect hemopoietic cells, for example, during chemotherapy or radiotherapy of solid tumours and/or accelerate the recovery of blood forming cells following chemotherapy or radiotherapy (e.g., of solid tumours). Reducing the levels of SHIP in hemopoietic cells enhances their proliferation and survival and significantly increases their resistance to chemotherapy-induced cell death. SHIP levels may be reduced using SHIP inhibitors, e.g., siRNA molecules selective for SHIP. Redaction of SHIP using siRNA increases the survival and/or proliferation of a wide range of hemopoietic cells, including platelets, and enhances the survival of hemopoietic cells during or following chemo- or radio-therapy.

### Hemopoietic Cells

By “hemopoietic” or “hematopoietic” is meant blood or blood cells formed by hematopoiesis or haemopoiesis in bone marrow and peripheral blood.

Hemopoietic Stem Cells (HSCs) are the most primitive cells present in the blood system and are capable of generating all of the cell populations present in the blood. HSCS are also capable of virtually indefinite self renewal (i.e., remaining a stem cell after cell division), and have the ability to choose between self-renewal and differentiation (ultimately, into a mature hemopoietic cell). HSCs also migrate in a regulated fashion, and are subject to regulation by apoptosis. HSCs are rare and are thought to account for an estimated 1 in 10,000 to 15,000 nucleated cells in the bone marrow, and an estimated 1 in 100,000 in the peripheral blood.

Hemopoietic Progenitor Cells (HPCs) are cells that are derived from and further differentiated from HSCs. When compared to HSCs, HPCs have a relatively reduced capacity to differentiate (they can generate only a subset of the possible lineages), although they are capable of extensive and rapid proliferation and can typically generate a large number of mature cells. Importantly, HPCs have a limited capacity to self-renew and therefore require regeneration from HSCs. A subset of HPCs can be held in a “pool” i.e., where the cells are not actively cycling. HPCs are generally present in larger numbers than HSCs and can therefore be more rapidly mobilized or expanded in the hemopoietic recovery process. HPCs include Common Lymphoid Progenitors (CLPs), which in adults, have the potential to generate all of we lymphoid but not the myeloerythroid cells, and Common Myeloid Progenitors (CMPs), which have the potential to generate all of the mature myeloerythroid cells, but not lymphoid cells.

HPCs give rise to the different blood cell types of the myeloid and lymphoid lineages. The myeloerythroid lineage includes granulocytes (neutrophils, eosinophils, basophils), mast cells, monocytes (histiocytes, macrophages, dendritic cells, Langerhans cells, microglia, Kupffer cells, osteoclasts), megakaryoblasts, megakaryocytes, erythrocytes, platelets and their various progenitors, e.g., colony forming units of the granulocytic/monocytic lineage (CFU-GM), burst forming units of the erythroid lineage (BFU-E), etc. The lymphoid lineage includes T-cells, B-cells, NK-cells and their progenitors, etc.

HSCs and/or HPCs may be obtained from bone marrow, or from peripheral blood upon pre-treatment with cytokines, such as granulocyte colony stimulating factor (G-CSF), which induces release of HSCs and/or HPCs from the bone marrow. HSCs and/or HPCs may also be obtained from umbilical cord blood, placenta, fetal liver or spleen, etc. Markers specific for HSCs and/or HPCs are known in the art, as are assays for detecting and isolating HSCs and/or HPCs and more differentiated hemopoietic cells. In alternative embodiments, HSCs are excluded from the methods and uses according to the invention. In alternative embodiments, the hemopoietic cell is a mature cell, a myeloid progenitor cell or a CMP. In alternative embodiments, the hemopoietic cell is a lymphoid cell, a lymphoid progenitor cell or a CLP.

Mature hemopoietic cells are terminally differentiated cells and include neutrophils, eosinophils, basophils, histiocytes, macrophages, dendritic cells, langerhans cells, microglia, Kupffer cells, osteoclasts, erythrocytes, platelets, T-cells, B-cells, and NK-cells. In alternative embodiments, lymphoid cells, e.g., NK cells, are excluded from the methods and uses according to the invention.

By “protecting a hemopoietic cell” or “enhancing the resistance of a hemopoietic cell” is meant increasing the survival of a hemopoietic cell, such as a hemopoietic progenitor cell or a mature hemopoietic cell, by for example decreasing cell death (e.g. by apoptosis). It is to be understood that decreasing cell death includes the prevention or slowing of cell death and may be partial, as long as the subject exhibits less cell death when compared with a control or reference subject, sample or compound. The increase in survival of the hemopoietic cell, or decrease in cell death, may be a change of any integer value between 10% and 90%, e.g., 10%, 20%, 30%, 40%, 50%, 60%, 70%, 80%, 90%, or may be over 100%, such as 200%, 300%, 500% or more, when compared with a control or reference subject, sample or compound. A control or reference subject, sample or compound may be a subject, sample or compound that has not been, or is not being, exposed to an inhibitor of a SH2-containing inositol-5′-phosphatase, or an inhibitor of SHIP1.

In alternative embodiments, “protecting a hemopoietic cell” or “enhancing the resistance of a hemopoietic cell” also includes increasing the proliferation of a hemopoietic cell, such as a hemopoietic progenitor cell or a mature hemopoietic cell. It is to be understood that the increase in cell proliferation may be partial, as long as the subject exhibits more cell proliferation when compared with a control or reference subject, sample or compound. The increase in proliferation of the hemopoietic cell may be a change of any integer value between 10% and 90%, e.g., 10%, 20%, 30%, 40%, 50%, 60%, 70%, 80%, 90%, or may be over 100%, such as 200%, 300%, 500% or more, when compared with a control or reference subject, sample or compound. A control or reference subject, sample or compound may be a subject, sample or compound that has not been, or is not being, exposed to an inhibitor of a SH2-containing inositol-5′-phosphatase, or an inhibitor of SHIP1.

Myelosuppression

Myelosuppression refers, in general, to a reduction in the production of blood cells. Myelosuppression therefore results in anemia, neutropenia, and thrombocytopenia.

Myelosuppression may result from a number of different factors, including stress, illness (such as cancer), drugs (such as chemotherapeutics), radiation therapy, infection (e.g., by HIV virus, other viruses or bacteria), environmental insults (such as accidental or deliberate exposure to chemicals, toxins, radiation, biological or chemical weapons), aging or other natural processes, etc.

Conventional treatments for myelosuppression include transfusion of blood, packed red blood cells, or platelets, or administration of growth factors such as erythropoietin, granulocyte colony stimulating factor (G-CSF), granulocyte-macrophage colony stimulating factor (GM-CSF), interleukin-11, etc.

Myeloablation generally refers to a severe form of myelosuppression that is typically induced by treatment with a regimen of chemotherapeutic agents, optionally combined with irradiation, that destroys host blood cells and bone marrow tissues. Myeloablation is used to prepare subjects for autologous or allogeneic bone marrow or stem cell transplantation, to prevent an undesired immune response of host cells against the graft cells, or to destroy aberrant cells, such as in leukemias and lymphomas. Full myeloablation refers to the complete destruction of host blood cells and bone marrow tissue. In general, the immune suppression or myelosuppression induced by standard chemotherapy or radiotherapy regimens do not result in full myeloablation. Accordingly, in alternative embodiments, myeloablation or full myeloablation is specifically excluded from the methods and uses according to the invention.

Immune suppression refers, in general, to a systemic reduction in immune function as evidenced by, for example, compromised in vitro proliferative response of B and T lymphocytes to mitogens, reduced natural killer (NK) cell cytotoxicity in vitro, reduced delayed type hypersensitivity (DTH) skin test responses to recall antigens. Immune suppression may result from a number of different factors, including stress, illness (such as cancer), drugs (such as chemotherapeutics), radiation therapy, infection (e.g., by HIV virus, other viruses or bacteria), transplantation (e.g., of bone marrow, or stem cells, or solid organs), environmental insults (such as accidental or deliberate exposure to chemicals, toxins, radiation, biological or chemical weapons), aging or other natural processes, etc.

### SH2-Containing Inositol-5′-Phosphatase (SHIP) Molecules

SH2-containing inositol-5′-phosphatases (or SH2-containing phosphatidylinositol phosphatase) are phosphatases that selectively remove the phosphate from the 5-position of the inositol ring in phosphoinositol-containing lipids.

The first such phosphatase identified, known as “SHIP” or “SHIP1,” is restricted to hemopoietic cells and is a 145 kDa protein that becomes both tyrosine phosphorylated and associated with the adaptor protein, Shc, after extracellular stimulation of hemopoietic cells. SHIP1 contains an N-terminal Src homology 2 (SH2) domain that binds preferentially to the amino acid sequence pY(Y/D)X(L/I/V), a centrally located 5′-phosphatase that selectively hydrolyses PI-3,4,5-P₃ and Ins(1,3,4,5)P₄(IP₄) in vitro, two NPXY amino acid sequences that, when phosphorylated, bind the phosphotyrosine binding (PTB) domains of Shc, Dok1 and Dok2 and a proline-rich C-terminus that binds a subset of Src homology 3 (SH3)-containing proteins. SHIP1 includes alternatively spliced forms (Lucas, D. M. and Rohrschneider, L. R. (1999) Blood 93, 1922-1933; Wolf, I., Lucas, D. M., Algate, P. A. and Rohrschneider, L. R. (2000) Genomics 69, 104-112) and C-terminal truncations (Damen, J. E., Liu, L., Ware, M. D., Ermolaeva, M., Majerus, P. W. and Krystal, G. (1998) Blood 92, 1199-1205). In alternative embodiments, SHIP1 includes, without limitation, alternative splice forms and truncations. In alternative embodiments, SHIP1 includes the sequences disclosed in U.S. Pat. No. 6,283,903 (issued to Krystal, May 29, 2001), PCT publication WO 97/10252 (naming Rohrschneider and Lioubin as inventors and published Mar. 20, 1997), or as set forth in SEQ ID NOs 1 to 4 or described in GenBank Accession Nos. U57650, U39203, U51742, NM_{—}001017915, or other SHIP1 mouse and human sequences, or SHIP1 sequences from other species.

A 104 kDa protein termed “stem cell SHIP” or “sSHIP” is only expressed in stem cells and HSCs (Tu, Z., Ninos, J. M., Ma, Z., Wang, J.-W., Lemos, M. P., Desponts, C, Ghansah, T., Howson, J. M. and Kerr, W. G. (2001) Blood 98, 2028-2038), but not in HPCs. sSHIP is generated by transcription from a promoter within the intron between exons 5 and 6 of the SHIP1 gene and is neither tyrosine phosphorylated nor associated with Shc following stimulation, but binds constitutively to Grb2. sSHIP is described in the GenBank Accession No. AF184912.

SHIP2, which is a more widely expressed 150 kDa protein that also becomes tyrosine phosphorylated and associated with Shc in response to extracellular stimulation, exists, like SHIP and sSHIP, in lower-molecular-mass forms and specifically hydrolyses the 5′-phosphate from PI-3,4,5-P₃ and IP₄ in vitro.

### SHIP Inhibitors

SHIP inhibitors include compounds that block SHIP function or SHIP levels directly or indirectly by, for example, targeting of a SHIP signal transduction pathway; inhibition of SHIP activation; inhibition of SHIP mRNA transcription; increased SHIP mRNA degradation; or inhibition of SHIP protein translation, stability or activity. In alternative embodiments, SHIP inhibitors include small molecules, such as LY288975 (Abstract #1225, Blood 98: p 291a, Nov. 16, 2001), antibodies or fragments thereof, such as humanized anti-SHIP1 antibodies, peptides and peptide fragments, such as SHIP1 dominant negative peptides and peptide fragments; ribozymes; and other nucleic acid molecules, including antisense oligonucleotides, shRNA, microRNA (miRNA) RNAi molecules, and siRNA molecules. In alternative embodiments, SHIP inhibitors include small molecules, such as LY288975 (Abstract #1225, Blood 98: p 291a, Nov. 16, 2001), antibodies or fragments thereof, such as humanized anti-SHIP1 antibodies, peptides and peptide fragments, such as SHIP1 dominant negative peptides and peptide fragments; ribozymes; and other nucleic acid molecules, shRNA, microRNA (miRNA)RNAi molecules, and siRNA molecules.

Polynucleotide-based inhibitors of SHIP may be single-stranded, double-stranded, or triplexes. In addition, they may be RNA, DNA, or contain both RNA and DNA. They may further include oligonucleotides and plasmids, including expression plasmids. In particular embodiments, expression plasmids express a polypeptide or polynucleotide inhibitor of SHIP, e.g., an siRNA, miRNA, shRNA or antisense oligonucleotide inhibitor of SHIP. In alternative embodiments, expression plasmids express a polypeptide or polynucleotide inhibitor of SHIP, e.g., an siRNA, miRNA, or shRNA. Additional SHIP inhibitors may be identified using commercially available libraries and standard screening and assay techniques. In alternative embodiments, SHIP inhibitors are not antisense oligonucleotide molecules.

In alternative embodiments, SHIP inhibitors specifically inhibit SHIP1, i.e., inhibit SHIP1 with a greater specificity when compared to inhibition of sSHIP, SHIP2, or other molecules. In particular embodiments, SHIP1-specific inhibitors reduce SHIP1 activity or expression to a level below 90%, below 80%, below 70%, below 60%, below 50%, below 40%, below 30%, below 20%, below 10%, below 5%, or below 2% as compared to SHIP1 activity or expression in the absence of said inhibitor. In related embodiments, SHIP 1-specific inhibitors do not significantly reduce the expression or activity of sSHIP, SHIP2, or other molecules. In particular embodiments, a SHIP1-specific inhibitor targets or binds a region of a SHIP1 protein or polynucleotide that is not present in a sSHIP or SHIP2 protein or polynucleotide. For example, a SHIP1-specific inhibitor may target the ATG sequence at the start of the coding region for SHIP1 or may target SHIP1 polypeptide or polynucleotide sequences corresponding to or encoding the approximately 300 bp SHIP1 SH2 domain, which follows the ATG region. In alternative embodiments, a SHIP1-specific inhibitor may target any sequence from positions 1 to 505 of SEQ ID NO: 1or 3, or may target SHIP1 polypeptide or polynucleotide sequences corresponding to or encoding the sequence from positions 1 to 505 of SEQ ID NO: 1 or 3.

RNA Interference and siRNA

Expression of a gene or coding or non-coding region of interest may be inhibited or prevented using RNA interference (RNAi) technology, a type of post-transcriptional gene silencing. RNAi may be used to create a functional “knockout”, i.e. a system in which the expression of a gene or coding or non-coding region of interest is reduced, resulting in an overall reduction of the encoded product. As such, RNAi may be performed to target a nucleic acid of interest or fragment or variant thereof, to in turn reduce its expression and the level of activity of the product which it encodes. Such a system may be used for functional studies of the product, as well as to treat disorders related to the activity of such a product. RNAi is described in for example Hammond S M, et al. (2001) Nature Rev Gen 2: 110-119, Sharp Pa. (2001) Genes Dev 15: 485-490, Caplen N J, et al. (2001) Proc. Natl. Acad. Sci. USA 98: 9746-9747 and published US patent applications 20020173478 (Gewirtz; published Nov. 21, 2002) and 20020132788 (Lewis et al.; published Nov. 7, 2002), all of which are herein incorporated by reference. Reagents and kits for performing RNAi are available commercially from for example Ambion Inc. (Austin, Tex., USA) and New England Biolabs hie. (Beverly, Mass., USA).

The initial agent for RNAi is a dsRNA molecule corresponding to a target nucleic acid. The dsRNA is then cleaved into short interfering RNAs (siRNAs) which are 21-23 nucleotides in length (19-21 bp duplexes, each with 2 nucleotide 3′ overhangs). The enzyme effecting this first cleavage step is referred to as “Dicer” and is categorized as a member of the RNase III family of dsRNA-specific ribonucleases. Alternatively, RNAi may be directly introduced into the cell, or generated within the cell by introducing into the cell a suitable precursor (e.g. vector) of such an siRNA or siRNA-like molecule. An siRNA may then associate with other intracellular components to form an RNA-induced silencing complex (RISC). The RISC thus formed may subsequently target a transcript of interest via base-pairing interactions between its siRNA component and the target transcript by virtue of homology, resulting in the cleavage of the target transcript approximately 12 nucleotides from the 3′ end of the siRNA. Thus the target mRNA is cleaved and the level of protein product it encodes is reduced.

RNAi may also be effected by the introduction of suitable in vitro synthesized siRNA or siRNA-like molecules into cells. RNAi may for example be performed using chemically-synthesized RNA (Brown D, et al. (2002) TechNotes 9: 3-5), for which suitable RNA molecules may be chemically synthesized using known methods. siRNA molecules may comprise two RNA strands, or they may comprise an RNA strand and a DNA strand, as described, e.g., in U.S Patent Application Publication No. 2004/0087526. Alternatively, suitable expression vectors may be used to transcribe such RNA either in vitro or in vivo. In vitro transcription of sense and antisense strands (encoded by sequences present on the same vector or on separate vectors) may be effected using for example T7 RNA polymerase, in which case the vector may comprise a suitable coding sequence operably-linked to a T7 promoter. The in vitro-transcribed RNA may in embodiments be processed (e.g. using E. coli RNase III) in vitro to a size conducive to RNAi. The sense and antisense transcripts combine to form an RNA duplex which is introduced into a target cell of interest. Other vectors may be used, which express short hairpin RNAs (shRNAs) which can be processed into siRNA-like molecules. Various vector-based methods are described in for example Brummelkamp T R, et al. (2002) Science 296:550-553, Lee N S, et al. (2002) Nature Biotechnol. 20:500-505, Miyagishi M, and Taira K. (2002) Nature Biotechnol. 20:497-500, Paddison P J, et al. (2002). Genes & Dev. 16:948-958, Paul C P, et al. (2002) Nature Biotechnol. 20:505-508, Sui G, et al. (2002) Proc. Natl. Acad. Sci. USA 99:5515-5520, and Yu J-Y, et al. (2002) Proc. Natl. Acad. Set. USA 99:6047-6052, all of which are herein incorporated by reference. Various methods for introducing such vectors into cells, either in vitro or in vivo (e.g. gene therapy) are known in the art.

Accordingly, SHIP expression may be inhibited by introducing into or generating within a cell an siRNA or siRNA-like molecule corresponding to a SHIP-encoding nucleic acid or fragment thereof, or to an nucleic acid homologous thereto. In particular embodiments, the siRNA specifically targets SHIP1. In various embodiments such a method may entail the direct administration of the siRNA or siRNA-like molecule into a cell, or use of the vector-based methods described above.

The present invention specifically provides siRNAs consisting of, consisting essentially of or comprising at least 15 or more contiguous nucleotides of one of the SHIP genes, particularly the SHIP1, sSHIP, or SHIP2 genes of any species, including human and mouse. In particular embodiments, the siRNA comprises less than 30 nucleotides per strand, e.g., 21-23 nucleotides. The double stranded siRNA agent can either have blunt ends or may have overhangs of 1-4 nucleotides from one or both 3′ ends of the agent. In an embodiment, siRNA or siRNA-like molecules comprise a 19-21 bp duplex portion, each strand having a 2 nucleotide 3′ overhang.

Further, the siRNA may contain additional modifications. For example, the siRNA may either contain only naturally occurring ribonucleotide subunits, or it can be synthesized to contain one or more modifications to the sugar or base of one or more of the ribonucleotide subunits that is included in the siRNA. The siRNA can be further modified so as to be attached to a ligand that is selected to improve stability, distribution or cellular uptake of the agent. One aspect of the present invention relates to a double-stranded siRNA comprising at least one non-natural nucleobase. In certain embodiments, the non-natural nucleobase is difluorotolyl, nitroindolyl, nitropyrrolyl, or nitroimidazolyl. In certain embodiments, only one of the two oligonucleotide strands of the double-stranded oligonucleotide contains a non-natural nucleobase. In certain embodiments, both of the oligonucleotide strands of the double-stranded oligonucleotide independently contain a non-natural nucleobase. Thus, in alternative embodiments, siRNA molecules may include a duplex having two strands and at least one modified nucleotide in the double-stranded region, where each strand is about 15 to about 60 nucleotides in length. Modified nucleotides suitable for use with siRNA are known.

siRNA molecules selective for a SHIP molecule may be determined using appropriate software programs, such as Promega (www.promega.com/siRNADesigner/program/); Whitehead (jura.wi.mit.edu/bioc/siRNAext/); Dharmacon (www.dharmacon.com/DesignCenter/DesignCenterPage.aspx); CSHL Jack Lin (www.ic.sunysb.edu/stu/shilin/rnai.html); Ambion (www.ambion.com/techlib/misc/siRNA finder.html); GeneScript (www-genscript.com/ssl-in/app/mai); Deqor (cluster-1.mpi-cbg.de/Deqor/deqor.html) by, for example, entering the human SHIP sequence into the query field of the search engine. In alternative embodiments, an siRNA molecule selective for SHIP1 includes one or more of the molecules listed in Table 1.

TABLE 1

| | | | |
|---|---|---|---|
| Promega | | | |
| GUUUACACUUACAGAAUUCUU | SEQ ID NO: 15 | UUCAAAUGUGAAUGUCUUAAG | SEQ ID NO: 16 |
| GUAUCGGAAUUGCGUUUACUU | SEQ ID NO: 17 | UUCAUAGCCUUAACGCAAAUG | SEQ ID NO: 18 |
| GGUGACCCAUCUGCAAUACUU | SEQ ID NO: 19 | UUCCACUGGGUAGACGUUAUG | SEQ ID NO: 20 |
| GGAAGUCAGUCAGUUAAGCUU | SEQ ID NO: 21 | UUCCUUCAGUCAGUCAAUUCG | SEQ ID NO: 22 |
| GAAUUGCGUUUACACUUACUU | SEQ ID NO: 23 | UUCUUAACGCAAAUGUGAAUG | SEQ ID NO: 24 |
| GUGACCCAUCUGCAAUACCUU | SEQ ID NO: 25 | UUCACUGGGUAGACGUUAUGG | SEQ ID NO: 26 |
| GCGCUCUGCGUGCUGUAUCUU | SEQ ID NO: 27 | UUCGCGAGACGCACGACAUAG | SEQ ID NO: 28 |
| GUUCCAGCGACUGCAAAGCUU | SEQ ID NO: 29 | UUCAAGGUCGCUGACGUUUCG | SEQ ID NO: 30 |
| GCAGCUCAGUUUCCUUUCCUU | SEQ ID NO: 31 | UUCGUCGAGUCAAAGGAAAGG | SEQ ID NO: 32 |
| GGCGGAGGAGCUGCUUUCCUU | SEQ ID NO: 33 | UUCCGCCUCCUCGACGAAAGG | SEQ ID NO: 34 |
| GAUGAUAAAUUCACUGUUCUU | SEQ ID NO: 35 | UUCUACUAUUUAAGUGACAAG | SEQ ID NO: 36 |
| GCCGCAGAAGAACCACUUCUU | SEQ ID NO: 37 | UUCGGCGUCUUCUUGGUGAAG | SEQ ID NO: 38 |
| GGAACCAUGGCAACAUCACUU | SEQ ID NO: 39 | UUCCUUGGUACCGUUGUAGUG | SEQ ID NO: 40 |
| GCGCCUGAAACAGGAAGUCUU | SEQ ID NO: 41 | UUCGCGGACUUUGUCCUUCAG | SEQ ID NO: 42 |
| GUGCCAGCGAGUCCAUCUCUU | SEQ ID NO: 43 | UUCACGGUCGCUCAGGUAGAG | SEQ ID NO: 44 |
| GUCCAUCUCCCGGGCAUACUU | SEQ ID NO: 45 | UUCAGGUAGAGGGCCCGUAUG | SEQ ID NO: 46 |
| GUACGCUGCGACCAGUUGCUU | SEQ ID NO: 47 | UUCAUGCGACGCUGGUCAACG | SEQ ID NO: 48 |
| GCACGGCCGCAGAAGAACCUU | SEQ ID NO: 49 | UUCGUGCCGGCGUCUUCUUGG | SEQ ID NO: 50 |
| GUAGUCCCAGUUGAGAAGCUU | SEQ ID NO: 51 | UUCAUCAGGGUCAACUCUUCG | SEQ ID NO: 52 |
| GCCUGCUAGGCCAUGCUUCUU | SEQ ID NO: 53 | UUCGGACGAUCCGGUACGAAG | SEQ ID NO: 54 |
| GGGUCCAGCAGUCUUCCUCUU | SEQ ID NO: 55 | UUCCCAGGUCGUCAGAAGGAG | SEQ ID NO: 56 |
| GGAGGACACAGAAAGUGUCUU | SEQ ID NO: 57 | UUCCUCCUGUGUCUUUCACAG | SEQ ID NO: 58 |
| GGGCUGGUGACCCAUCUGCUU | SEQ ID NO: 59 | UUCCCGACCACUGGGUAGACG | SEQ ID NO: 60 |
| GAACCAUGGCAACAUCACCUU | SEQ ID NO: 61 | UUCUUGGUACCGUUGUAGUGG | SEQ ID NO: 62 |
| GGGCUUCCAGAAGAGCAUCUU | SEQ ID NO: 63 | UUCCCGAAGGUCUUCUCGUAG | SEQ ID NO: 64 |
| GCGACUGCAAAGCAUGGACUU | SEQ ID NO: 65 | UUCGCUGACGUUUCGUACCUG | SEQ ID NO: 66 |
| GCGUGCCAGCGAGUCCAUCUU | SEQ ID NO: 67 | UUCGCACGGUCGCUCAGGUAG | SEQ ID NO: 68 |
| GGACACUUCAAUUGUACGCUU | SEQ ID NO: 69 | UUCCUGUGAAGUUAACAUGCG | SEQ ID NO: 70 |
| GAAAGUGUCGUGUCUCCACUU | SEQ ID NO: 71 | UUCUUUCACAGCACAGAGGUG | SEQ ID NO: 72 |
| GGCAAGGACGGGAGCUUCCUU | SEQ ID NO: 73 | UUCCGUUCCUGCCCUCGAAGG | SEQ ID NO: 74 |
| GAUUAUUUAAGCACUCAGCUU | SEQ ID NO: 75 | UUCUAAUAAAUUCGUGAGUCG | SEQ ID NO: 76 |
| GUCCAGCAGUCUUCCUCACUU | SEQ ID NO: 77 | UUCAGGUCGUCAGAAGGAGUG | SEQ ID NO: 78 |
| GAAACUGACCACACUGCUCUU | SEQ ID NO: 79 | UUCUUUGACUGGUGUGACGAG | SEQ ID NO: 80 |
| GAGGCCACCAAGAGGCAACUU | SEQ ID NO: 81 | UUCUCCGGUGGUUCUCCGUUG | SEQ ID NO: 82 |
| GUCUAGGGCAUGGCAUCCCUU | SEQ ID NO: 83 | UUCAGAUCCCGUACCGUAGGG | SEQ ID NO: 84 |
| GAGCAUCUUAAGGCCAUCCUU | SEQ ID NO: 85 | UUCUCGUAGAAUUCCGGUAGG | SEQ ID NO: 86 |
| GGCCUGUCUAGGGCAUGGCUU | SEQ ID NO: 87 | UUCCGGACAGAUCCCGUACCG | SEQ ID NO: 88 |
| GAAGUGGCCACAACUCUCCUU | SEQ ID NO: 89 | UUCUUCACCGGUGUUGAGAGG | SEQ ID NO: 90 |
| GUUCUUCACCAAGCUGGACUU | SEQ ID NO: 91 | UUCAAGAAGUGGUUCGACCUG | SEQ ID NO: 92 |
| GUGAGGCCAAGGAGGUUCCUU | SEQ ID NO: 93 | UUCACUCCGGUUCCUCCAAGG | SEQ ID NO: 94 |
| GAAACAUCCCGCUGACUGCUU | SEQ ID NO: 95 | UUCUUUGUAGGGCGACUGACG | SEQ ID NO: 96 |
| GGCAUCCGAAGGCGUCUCCUU | SEQ ID NO: 97 | UUCCGUAGGCUUCCGCAGAGG | SEQ ID NO: 98 |
| GGCAUCCCACGUGGGUGUCUU | SEQ ID NO: 99 | UUCCGUAGGGUGCACCCACAG | SEQ ID NO: 100 |
| GACUGCAAAGCAUGGACACUU | SEQ ID NO: 101 | UUCUGACGUUUCGUACCUGUG | SEQ ID NO: 102 |
| GCCGCUGGAGGAAGAGGACUU | SEQ ID NO: 103 | UUCGGCGACCUCCUUCUCCUG | SEQ ID NO: 104 |
| GAGGACACAGGCGACGACCUU | SEQ ID NO: 105 | UUCUCCUGUGUCCGUGCUGG | SEQ ID NO: 106 |
| GAGACAUUGUUCCAGCGACUU | SEQ ID NO: 107 | UUCUCUGUAACAAGGUCGCUG | SEQ ID NO: 108 |
| GACGGGAGCUUCCUCGUGCUU | SEQ ID NO: 109 | UUCUGCCCUCGAAGGAGCACG | SEQ ID NO: 110 |
| GUGUCUCCACCCGAGCUGCUU | SEQ ID NO: 111 | UUCACAGAGGUGGGCUCGACG | SEQ ID NO: 112 |
| GAGGGCUGGCAAGAGAGCCUU | SEQ ID NO: 113 | UUCUCCCGACCGUUCUCUCGG | SEQ ID NO: 114 |
| GCUGCUUUCCAGGACAGGCUU | SEQ ID NO: 115 | UUCGACGAAAGGUCCUGUCCG | SEQ ID NO: 116 |
| GGAGGAAGAGGACACAGGCUU | SEQ ID NO: 117 | UUCCUCCUUCUCCUGUGUCCG | SEQ ID NO: 118 |
| GAGGGAGAGCAGAAGGCUCUU | SEQ ID NO: 119 | UUCUCCCUCUCGUCUUCCGAG | SEQ ID NO: 120 |
| GCUCAGUUUCCUUUCCCUCUU | SEQ ID NO: 121 | UUCGAGUCAAAGGAAAGGGAG | SEQ ID NO: 122 |
| GCCAGCGAGUCCAUCUCCCUU | SEQ ID NO: 123 | UUCGGUCGCUCAGGUAGAGGG | SEQ ID NO: 124 |
| GAAGAACCACUUCUCUGGCUU | SEQ ID NO: 125 | UUCUUCUUGGUGAAGAGACCG | SEQ ID NO: 126 |
| GAGCUUCCUCGUGCGUGCCUU | SEQ ID NO: 127 | UUCUCGAAGGAGCACGCACGG | SEQ ID NO: 128 |
| GUCGUGUCUCCACCCGAGCUU | SEQ ID NO: 129 | UUCAGCACAGAGGUGGGCUCG | SEQ ID NO: 130 |
| GCAUACGCGCUCUGCGUGCUU | SEQ ID NO: 131 | UUCGUAUGCGCGAGACGCACG | SEQ ID NO: 132 |
| GGAGCUUCCUCGUGCGUGCUU | SEQ ID NO: 133 | UUCCUCGAAGGAGCACGCACG | SEQ ID NO: 134 |
| GACGUCCUCAGAGCCGGUCUU | SEQ ID NO: 135 | UUCUGCAGAGGUCUCGGCCAG | SEQ ID NO: 136 |
| GGAAGAGGACACAGGCGACUU | SEQ ID NO: 137 | UUCCUUCUCCUGUGUCCGCUG | SEQ ID NO: 138 |
| GGAGGUUCAGGGUGGGUGCUU | SEQ ID NO: 139 | UUCCUCCAAGUCCCACCCACG | SEQ ID NO: 140 |
| GACAGGCAAGGACGGGAGCUU | SEQ ID NO: 141 | UUCUGUCCGUUCCUGCCCUCG | SEQ ID NO: 142 |
| Whitehead | | | |
| S 5′:GCUGGACCAGCUCAUCGAGdTdT | SEQ ID NO: 143 | cDNA:AAGCTGGACCAGCTCATCGAGTTAS | SEQ ID NO: 144 |
| 3′:TdTdCGACCUGGUCGAGUAGCUC | SEQ ID NO: 145 | S 5′-AGCAUGGACACCAGUGGGCdTdT | SEQ ID NO: 146 |
| cDNA:AAAGCATGGACACCAGTGGGCTTAS | SEQ ID NO: 147 | 3′:TdTdUCGUACCUGUGGUCACCCG | SEQ ID NO: 148 |
| Dharmacon Sense Strand Sequence | | | |
| GCAAGGAGCTCTATGGGTA | SEQ ID NO: 149 | GGAATTGCGTTTACACTTA | SEQ ID NO: 150 |
| GGAGAGGGCTGGCAAGAGA | SEQ ID NO: 151 | GCCCAATGAAGATGATAAA | SEQ ID NO: 152 |
| ACAGGAAGTCAGTCAGTTA | SEQ ID NO: 153 | GCGTTTACACTTACAGAAT | SEQ ID NO: 154 |
| AGACATTGTTCCAGCGACT | SEQ ID NO: 155 | CAAGGAGCTCTATGGGTAA | SEQ ID NO: 156 |
| GGAAGGAGAGGGCTGGCAA | SEQ ID NO: 157 | CCTGAGGAGGACACAGAAA | SEQ ID NO: 158 |
| TGAAACAGGAAGTCAGTCA | SEQ ID NO: 159 | CCATGAGGTTCTTCACCAA | SEQ ID NO: 160 |
| GGACCAGCTCATCGAGTTT | SEQ ID NO: 161 | TCACTGAGCGCCTGAAACA | SEQ ID NO: 162 |
| CCGTAGTCCCAGTTGAGAA | SEQ ID NO: 163 | CTGTATCGGAATTGCGTTT | SEQ ID NO: 164 |
| CGGAATTGCGTTTACACTT | SEQ ID NO: 165 | AGGAAGAGGACACAGGCGA | SEQ ID NO: 166 |
| CCAGTTGCCAGGAAGGAGA | SEQ ID NO: 167 | CAGGAAGTCAGTCAGTTAA | SEQ ID NO: 168 |
| TGCCCAATGAAGATGATAA | SEQ ID NO: 169 | TGGTTTCTGTAATGAGGAA | SEQ ID NO: 170 |
| CGTTTACACTTACAGAATT | SEQ ID NO: 171 | GTTTACACTTACAGAATTC | SEQ ID NO: 172 |
| TCATCGAGTTTTACAAGAA | SEQ ID NO: 173 | CTGTGCCGCTGGAGGAAGA | SEQ ID NO: 174 |
| CCAAGAAACATCCCGCTGA | SEQ ID NO: 175 | CGCCCAGGACTCTGAATTT | SEQ ID NO: 176 |
| ACTCTGAATTTGTGAAGAC | SEQ ID NO: 177 | CAGGCAAGGACGGGAGCTT | SEQ ID NO: 178 |
| CATGGACACCAGTGGGCTT | SEQ ID NO: 179 | CTTAAGGCCATCCAAGATT | SEQ ID NO: 180 |
| CCAAGATTATTTAAGCACT | SEQ ID NO: 181 | CAAGATTATTTAAGCACTC | SEQ ID NO: 182 |
| GAATTCTGCCCAATGAAGA | SEQ ID NO: 183 | TGAGGAGGACACAGAAAGT | SEQ ID NO: 184 |
| CACAGAAAGTGTCGTGTCT | SEQ ID NO: 185 | TTAAGGCCATCCAAGATTA | SEQ ID NO: 186 |
| TGAAGAAACTGACCACACT | SEQ ID NO: 187 | GCTGGTGACCCATCTGCAA | SEQ ID NO: 188 |
| GGAAGAGGACACAGGCGAC | SEQ ID NO: 189 | CCTGTGAGGCCAAGGAGGT | SEQ ID NO: 190 |
| CTGAAGAAACTGACCACAC | SEQ ID NO: 191 | TGACCACACTGCTCTGCAA | SEQ ID NO: 182 |
| GTTTCTGTAATGAGGAAGT | SEQ ID NO: 193 | CCTGCTGGAACCATGGCAA | SEQ ID NO: 194 |
| GGAGCTGCTTTCCAGGACA | SEQ ID NO: 195 | CGACTGCAAAGCATGGACA | SEQ ID NO: 196 |
| CTGCAAAGCATGGACACCA | SEQ ID NO: 197 | | |
| Jack Lin | | | |
| AAAGCATGGACACCAGTGGGCTT | SEQ ID NO: 198 | AAGCTGGACCAGCTCATCGAGTT | SEQ ID NO: 199 |
| AAGCTGTGCCCCCTTGGGTGTTT | SEQ ID NO: 200 | AAGAAACTGACCACACTGCTCTG | SEQ ID NO: 201 |
| AAGGCGTCTCCATGAGGTTCTTC | SEQ ID NO: 202 | AATGAGGAAGTTCTCCGCAGCTC | SEQ ID NO: 203 |
| AAGCATGGACACCAGTGGGCTTC | SEQ ID NO: 204 | AAACATGGGGCTGGTGACCCATC | SEQ ID NO: 205 |
| AACCATGGCAACATCACCCGCTC | SEQ ID NO: 206 | AACATCCCGCTGACTGCCAGCTC | SEQ ID NO: 207 |
| AAGAGGCAACGGGCGGCAGGTTG | SEQ ID NO: 208 | AAGATTATTTAAGCACTCAGCTC | SEQ ID NO: 209 |
| AATTCTGCCCAATGAAGATGATA | SEQ ID NO: 210 | GAAGGCGTCTCCATGAGGTTCTT | SEQ ID NO: 211 |
| CAGCTCGCCCAGGACTCTGAATT | SEQ ID NO: 212 | CAGTTGAGAAGCTGTGCCCCCTT | SEQ ID NO: 213 |
| GAAGCTGTGCCCCCTTGGGTGTT | SEQ ID NO: 214 | CAAGAGGCAACGGGCGGCAGGTT | SEQ ID NO: 215 |
| CAGGGCCCCCCCCTCTCTCTCTT | SEQ ID NO: 216 | CAGGAAGTCAGTCAGTTAAGCTG | SEQ ID NO: 217 |
| CAATTGTACGCTGCGACCAGTTG | SEQ ID NO: 218 | CAGAAGAGCATCTTAAGGCCATC | SEQ ID NO: 219 |
| GAAGTCAGTCAGTTAAGCTGGTG | SEQ ID NO: 220 | GAGGCCAAGGAGGTTCCTTTTTC | SEQ ID NO: 221 |
| GAGGACACAGAAAGTGTCGTGTC | SEQ ID NO: 222 | GAAGTTCTCCGCAGCTCAGTTTC | SEQ ID NO: 223 |
| TAGGCCATGCTTCTTCAGAAGTG | SEQ ID NO: 224 | CAGAATTCTGCCCAATGAAGATG | SEQ ID NO: 225 |
| CACTTACAGAATTCTGCCCAATG | SEQ ID NO: 226 | CAGAAGTGGCCACAACTCTCCTG | SEQ ID NO: 227 |
| CAGTGGGCTTCCAGAAGAGCATC | SEQ ID NO: 228 | GAAGACAGGGTCCAGCAGTCTTC | SEQ ID NO: 229 |
| CATGGTCCCCTGCTGGAACCATG | SEQ ID NO: 230 | CACGGCCGCAGAAGAACCACTTC | SEQ ID NO: 231 |
| CATCTTAAGGCCATCCAAGATTA | SEQ ID NO: 232 | GACAGGGTCCAGCAGTCTTCCTC | SEQ ID NO: 233 |
| GATAAATTCACTGTTCAGGCATC | SEQ ID NO: 234 | GACGTCTCCAGAGCCGGTCATTC | SEQ ID NO: 235 |
| CACTCAGCTCGCCCAGGACTCTG | SEQ ID NO: 236 | CAGGGGGACTTCAGCTGCCACTG | SEQ ID NO: 237 |
| CATCCAAGATTATTTAAGCACTC | SEQ ID NO: 238 | CAGGCAAGGACGGGAGCTTCCTC | SEQ ID NO: 239 |
| CAGAAGGCTCGGGGGCCTGTCTA | SEQ ID NO: 240 | GAGAAGCTGTGCCCCCTTGGGTG | SEQ ID NO: 241 |
| TATCGGAATTGCGTTTACACTTA | SEQ ID NO: 242 | CAATGAAGATGATAAATTCACTG | SEQ ID NO: 243 |
| GAGACCAGCCGGCCGAGCCTCTC | SEQ ID NO: 244 | GACGGCCAGGGCCCCCCCCTCTC | SEQ ID NO: 245 |
| Ambion | | | |
| AAGCTGTGCCCCCTTGGGTGT | SEQ ID NO: 246 | AAGCCCTGAGGGAGAGCAGAA | SEQ ID NO: 247 |
| AAGGCTCGGGGGCCTGTCTAG | SEQ ID NO: 248 | AAGAACCACTTCTCTGGCCCA | SEQ ID NO: 249 |
| AACCACTTCTCTGGCCCACCC | SEQ ID NO: 250 | AAGTGGCCACAACTCTCCTGA | SEQ ID NO: 251 |
| AACTCTCCTGACGTCTCCAGA | SEQ ID NO: 252 | AATTGTACGCTGCGACCAGTT | SEQ ID NO: 253 |
| AAGGAGAGGGCTGGCAAGAGA | SEQ ID NO: 254 | AAGAGAGCCGCGGCAGCCGTG | SEQ ID NO: 255 |
| AATGAGGAAGTTCTCCGCAGC | SEQ ID NO: 256 | AAGTTCTCCGCAGCTCAGTTT | SEQ ID NO: 257 |
| AAACAGGAAGTCAGTCAGTTA | SEQ ID NO: 258 | AAGTCAGTCAGTTAAGCTGGT | SEQ ID NO: 259 |
| AAGCTGGTGGCAGCAGCCGAG | SEQ ID NO: 260 | AAGAGGCAACGGGCGGCAGGT | SEQ ID NO: 261 |
| AACGGGCGGCAGGTTGCAGTG | SEQ ID NO: 262 | AACCATGGCAACATCACCCGC | SEQ ID NO: 263 |
| AACATCACCCGCTCCAAGGCG | SEQ ID NO: 264 | AAGGCGGAGGAGCTGCTTTCC | SEQ ID NO: 265 |
| AAGGACGGGAGCTTCCTCGTG | SEQ ID NO: 266 | AATTGCGTTTACACTTACAGA | SEQ ID NO: 267 |
| AATTCTGCCCAATGAAGATGA | SEQ ID NO: 268 | AATGAAGATGATAAATTCACT | SEQ ID NO: 269 |
| AAGATGATAAATTCACTGTTC | SEQ ID NO: 270 | AAATTCACTGTTCAGGCATCC | SEQ ID NO: 271 |
| AAGGCGTCTCCATGAGGTTCT | SEQ ID NO: 272 | AAGCTGGACCAGCTCATCGAG | SEQ ID NO: 273 |
| AACATGGGGCTGGTGACCCAT | SEQ ID NO: 274 | AATACCCTGTGCCGCTGGAGG | SEQ ID NO: 275 |
| AAGAGGACACAGGCGACGACC | SEQ ID NO: 276 | AAAGTGTCGTGTCTCCACCCG | SEQ ID NO: 277 |
| AAGAAACATCCCGCTGACTGC | SEQ ID NO: 278 | AAACATCCCGCTGACTGCCAG | SEQ ID NO: 279 |
| AAACGAGAATCCCCGAGCGAC | SEQ ID NO: 280 | AATCCCCGAGCGACCGAGACC | SEQ ID NO: 281 |
| AAAGCATGGACACCAGTGGGC | SEQ ID NO: 282 | AAGAGCATCTTAAGGCCATCC | SEQ ID NO: 283 |
| AAGGCCATCCAAGATTATTTA | SEQ ID NO: 284 | AAGATTATTTAAGCACTCAGC | SEQ ID NO: 285 |
| AAGCACTCAGCTCGCCCAGGA | SEQ ID NO: 286 | AATTTGTGAAGACAGGGTCCA | SEQ ID NO: 287 |
| AAGACAGGGTCCAGCAGTCTT | SEQ ID NO: 288 | AAGAAACTGACCACACTGCTC | SEQ ID NO: 289 |
| AAACTGACCACACTGCTCTGC | SEQ ID NO: 290 | | |
| Genscript | | | |
| GTCGGTTTCTATCTACTTAAA | SEQ ID NO: 291 | TACGAAAGGACAAGAGAATTA | SEQ ID NO: 292 |
| GCTAAGACTTCACCAGTCAAA | SEQ ID NO: 293 | ATAACTTGACCAACGGAACAA | SEQ ID NO: 294 |
| ATCCTATTCTAGAGTCCATAT | SEQ ID NO: 295 | CCAATGGTGCAGCCGCTATTA | SEQ ID NO: 296 |
| TCTGAGTTCAGACCGGAGTAA | SEQ ID NO: 297 | CAGTCAAAGCGAACTACTATA | SEQ ID NO: 298 |
| CGCTATTAAAGGTTCGTTTGT | SEQ ID NO: 299 | GAGTAATCCAGGTCGGTTTCT | SEQ ID NO: 300 |
| Deqor | | | |
| GACTCTGAATTTGTGAAGACA | SEQ ID NO: 301 | TCCTGAGACTTAAACACTTCT | SEQ ID NO: 302 |
| (->sense) | | (<-antisense) | |
| CTTTCTCTCTCTCTCTCTTGC | SEQ ID NO: 303 | GAGAAAGAGAGAGAGAGAGAA | SEQ ID NO: 304 |
| (->sense) | | (<-antisense) | |
| CTTAAGGCCATCCAAGATTAT | SEQ ID NO: 305 | TAGAATTCCGGTAGGTTCTAA | SEQ ID NO: 306 |
| (->sense) | | (<-antisense) | |
| CACCTGAAGAAACTGACCACA | SEQ ID NO: 307 | GAGTGGACTTCTTTGACTGGT | SEQ ID NO: 308 |
| (->sense) | | (<-antisense) | |
| CTCTCTCTCTTGCTTGGTTTC | SEQ ID NO: 309 | GAGAGAGAGAGAACGAACCAA | SEQ ID NO: 310 |
| (->sense) | | (<-antisense) | |
| GCGTTTACACTTACAGAATTC | SEQ ID NO: 311 | AACGCAAATGTGAATGTCTTA | SEQ ID NO: 312 |
| (->sense) | | (<-antisense) | |
| CGTTTACACTTACAGAATTCT | SEQ ID NO: 313 | ACGCAAATGTGAATGTCTTAA | SEQ ID NO: 314 |
| (->sense) | | (<-antisense) | |
| GCCATCCAAGATTATTTAAGC | SEQ ID NO: 315 | TCCGGTAGGTTCTAATAAATT | SEQ ID NO: 316 |
| (->sense) | | (<-antisense) | |
| CCTGAAGAAACTGACCACACT | SEQ ID NO: 317 | GTGGACTTCTTTGACTGGTGT | SEQ ID NO: 318 |
| (->sense) | | (<-antisense) | |
| CAGGACTCTGAATTTGTGAAG | SEQ ID NO: 319 | GGGTCCTGAGACTTAAACACT | SEQ ID NO: 320 |
| (->sense) | | (<-antisense) | |
| Cross silencers | | | |
| TCTCTCTCTCTCTCTTGCTTG | SEQ ID NO: 321 | AAAGAGAGAGAGAGAGAACGA | SEQ ID NO: 322 |
| (->sense) | | (<-antisense) | |
| TGCCCAATGAAGATGATAAAT | SEQ ID NO: 323 | AGACGGGTTACTTCTACTATT | SEQ ID NO: 324 |
| (->sense) | | (<-antisense) | |
| GGAGGAGCTGCTTTCCAGGAC | SEQ ID NO: 325 | CGCCTCCTCGACGAAAGGTCC | SEQ ID NO: 326 |
| (->sense) | | (<-antisense) | |
| CCCCCCTCTCTCTCTTTCTCT | SEQ ID NO: 327 | GGGGGGGGAGAGAGAGAAAGA | SEQ ID NO: 328 |
| (->sense) | | (<-antisense) | |
| AGTTTCCTTTCCCTCACTGAG | SEQ ID NO: 329 | AGTCAAAGGAAAGGGAGTGAC | SEQ ID NO: 330 |
| (->sense) | | (<-antisense) | |
| CCCCCTCTCTCTCTTTCTCTC | SEQ ID NO: 331 | GGGGGGGAGAGAGAGAAAGAG | SEQ ID NO: 332 |
| (->sense) | | (<-antisense) | |
| GGTGGTGTGTGGGTCCTGGGG | SEQ ID NO: 333 | AGCCACCACACACCCAGGACC | SEQ ID NO: 334 |
| (->sense) | | (<-antisense) | |
| CCGAGGAGGCCCACGCCCACC | SEQ ID NO: 335 | CCGGCTCCTCCGGGTGCGGGT | SEQ ID NO: 336 |
| (->sense) | | (<-antisense) | |
| CCCTCTCTCTCTTTCTCTCTC | SEQ ID NO: 337 | GGGGGAGAGAGAGAAAGAGAG | SEQ ID NO: 338 |
| (->sense) | | (<-antisense) | |
| GGCCGAGGAGGCCCACGCCCA | SEQ ID NO: 339 | GGCCGGCTCCTCCGGGTGCGG | SEQ ID NO: 340 |
| (->sense) | | (<-antisense) | |
| GCCGAGGAGGCCCACGCCCAC | SEQ ID NO: 341 | GCCGGCTCCTCCGGGTGCGGG | SEQ ID NO: 342 |
| (->sense) | | (<-antisense) | |
| TCTTTCTCTCTCTCTCTCTTG | SEQ ID NO: 343 | AGAGAAAGAGAGAGAGAGAGA | SEQ ID NO: 344 |
| (->sense) | | (<-antisense) | |
| CCTCTCTCTCTTTCTCTCTCT | SEQ ID NO: 345 | GGGGAGAGAGAGAAAGAGAGA | SEQ ID NO: 346 |
| (->sense) | | (<-antisense) | |
| CTCTCTCTCTTTCTCTCTCTC | SEQ ID NO: 347 | GGGAGAGAGAGAAAGAGAGAG | SEQ ID NO: 348 |
| (->sense) | | (<-antisense) | |
| CTCTTTCTCTCTCTCTCTCTT | SEQ ID NO: 349 | GAGAGAAAGAGAGAGAGAGAG | SEQ ID NO: 350 |
| (->sense) | | (<-antisense) | |
| TCTCTCTCTTTCTCTCTCTCT | SEQ ID NO: 351 | GGAGAGAGAGAAAGAGAGAGA | SEQ ID NO: 352 |
| (->sense) | | (<-antisense) | |
| CTCTCTCTTTCTCTCTCTCTC | SEQ ID NO: 353 | GAGAGAGAGAAAGAGAGAGAG | SEQ ID NO: 354 |
| (->sense) | | (<-antisense) | |
| TCTCTCTTTCTCTCTCTCTCT | SEQ ID NO: 355 | AGAGAGAGAAAGAGAGAGAGA | SEQ ID NO: 356 |
| (->sense) | | (<-antisense) | |
| CTCTCTTTCTCTCTCTCTCTC | SEQ ID NO: 357 | GAGAGAGAAAGAGAGAGAGAG | SEQ ID NO: 358 |
| (->sense) | | (<-antisense) | |
| TCTCTTTCTCTCTCTCTCTCT | SEQ ID NO: 359 | AGAGAGAAAGAGAGAGAGAGA | SEQ ID NO: 360 |
| (->sense) | | (<-antisense) | |

In alternative embodiments, the siRNA or siRNA-like molecule is substantially identical to a SHIP-encoding nucleic acid or a fragment or variant (or a fragment of a variant) thereof. In alternative embodiments, the sense strand of the siRNA or siRNA-like molecule is substantially identical to SEQ ID NOs: 1 or 3 or a fragment thereof (RNA having U in place of T residues of the DNA sequence). In alternative embodiments, the siRNA molecule targeting SHIP with the sequence AAGAGTCAGGAAGGAGAGAAT (SEQ ID NO: 10) or AAGAGTCAGGAAGGAGAAAAT (SEQ ID NO: 11) is used to treat myelosuppression.

In alternative embodiments, a RNA interference, shRNA or siRNA molecule selective for SHIP 1 includes one or more of the sequences listed in Table 2. Table 3 lists sequences specific for human SHIP1.

TABLE 2

| Mature | | | |
|---|---|---|---|
| product | | Hairpin* | |
| CCCATATC | SEQ ID | TGCTGTTGACAGTGAGCGAGCCCATATC | SEQ ID |
| ACCCAAGA | NO: | ACCCAAGAAGTTTAGTGAAG | NO: |
| AGT | 361 | CCACAGATGTAAACTTCTTGGGTGATAT | 362 |
| | | GGGCGTGCCTACTGCCTCGGA | |
| GCTTCCAG | SEQ ID | TGCTGTTGACAGTGAGCGAGGCTTCCA | SEQ ID |
| AAGAGCAT | NO: | GAAGAGCATCTTATAGTGAAG | NO: |
| CTT | 363 | CCACAGATGTATAAGATGCTCTTCTGGA | 364 |
| | | AGCCCTGCCTACTGCCTCGGA | |
| CATATCCT | SEQ ID | TGCTGTTGACAGTGAGCGCGCATATCCT | SEQ ID |
| GATCAGCA | NO: | GATCAGCATTAATAGTGAAG | NO: |
| TTA | 365 | CCACAGATGTATTAATGCTGATCAGGAT | 366 |
| | | ATGCTTGCCTACTGCCTCGGA | |
| CTGTATCG | SEQ ID | TGCTGTTGACAGTGAGCGCGCTGTATCG | SEQ ID |
| GAATTGCG | NO: | GAATTGCGTTTATAGTGAAGC | NO: |
| TTT | 367 | CACAGATGTATAAACGCAATTCCGATA | 368 |
| | | CAGCATGCCTACTGCCTCGGA | |
| CTTATGAG | SEQ ID | TGCTGTTGACAGTGAGCGCGCTTATGA | SEQ ID |
| GATGGAAG | NO: | GGATGGAAGGAATTAGTGAAG | NO: |
| GAA | 369 | CCACAGATGTAATTCCTTCCATCCTCAT | 370 |
| | | AAGCTTGCCTACTGCCTCGGA | |
| CTGCTTTC | SEQ ID | TGCTGTTGACAGTGAGCGCGCTGCTTTC | SEQ ID |
| CAGGACAG | NO: | CAGGACAGGCAATAGTGAAGC | NO: |
| GCA | 371 | CACAGATGTATTGCCTGTCCTGGAAAGC | 372 |
| | | AGCTTGCCTACTGCCTCGGA | |
| CTGAAAGC | SEQ ID | TGCTGTTGACAGTGAGCGCCCTGAAAG | SEQ ID |
| CATCCAGG | NO: | CCATCCAGGATTATAGTGAAG | NO: |
| ATT | 373 | CCACAGATGTATAATCCTGGATGGCTTT | 374 |
| | | CAGGTTGCCTACTGCCTCGGA | |
| CCGCCCAT | SEQ ID | TGCTGTTGACAGTGAGCGCGCCGCCCAT | SEQ ID |
| ATCACCCA | NO: | ATCACCCAAGAATAGTGAAGC | NO: |
| AGA | 375 | CACAGATGTATTCTTGGGTGATATGGGC | 376 |
| | | GGCTTGCCTACTGCCTCGGA | |
| GTGCGTGC | SEQ ID | TGCTGTTGACAGTGAGCGCCGTGCGTG | SEQ ID |
| CAGCGAGT | NO: | CCAGCGAGTCCATTAGTGAAGC | NO: |
| CCA | 377 | CACAGATGTAATGGACTCGCTGGCACG | 378 |
| | | CACGATGCCTACTGCCTCGGA | |
| CTCTGCGT | SEQ ID | TGCTGTTGACAGTGAGCGAGCTCTGCGT | SEQ ID |
| GCTGTATC | NO: | GCTGTATCGGAATAGTGAAGC | NO: |
| GGA | 379 | CACAGATGTATTCCGATACAGCACGCA | 380 |
| | | GAGCGTGCCTACTGCCTCGGA | |
| CTCATTAA | SEQ ID | TGCTGTTGACAGTGAGCGCGCTCATTAA | SEQ ID |
| GTCACAGA | NO: | GTCACAGAAATTTAGTGAAG | NO: |
| AAT | 381 | CCACAGATGTAAATTTCTGTGACTTAAT | 382 |
| | | GAGCTTGCCTACTGCCTCGGA | |
| CGAGTCCT | SEQ ID | TGCTGTTGACAGTGAGCGCCCGAGTCCT | SEQ ID |
| CTGGAAGT | NO: | CTGGAAGTCTTATAGTGAAGC | NO: |
| CTT | 383 | CACAGATGTATAAGACTTCCAGAGGAC | 384 |
| | | TCGGTTGCCTACTGCCTCGGA | |
| GAGTCCAT | SEQ ID | TGCTGTTGACAGTGAGCGACGAGTCCA | SEQ ID |
| CTCCCGGG | NO: | TCTCCCGGGCATATAGTGAAG | NO: |
| CAT | 385 | CCACAGATGTATATGCCCGGGAGATGG | 386 |
| | | ACTCGCTGCCTACTGCCTCGGA | |
| GAGACACT | SEQ ID | TGCTGTTGACAGTGAGCGCGGAGAGAC | SEQ ID |
| CTTCCCAA | NO: | TCTTCCCAAGCTATAGTGAAG | NO: |
| GCT | 387 | CCACAGATGTATAGCTTGGGAAGAGTC | 388 |
| | | TCTCCATGCCTACTGCCTCGGA | |
| CGGGATGA | SEQ ID | TGCTGTTGACAGTGAGCGCGCGGGATG | SEQ ID |
| ATCCAGTG | NO: | AATCCAGTGGAATTAGTGAAG | NO: |
| GAA | 389 | CCACAGATGTAATTCCACTGGATTCATC | 390 |
| | | CCGCTTGCCTACTGCCTCGGA | |
| CCGAGCCT | SEQ ID | TGCTGTTGACAGTGAGCGAGCCGAGCC | SEQ ID |
| CTCCGAGA | NO: | TCTCCGAGACATTTAGTGAAGC | NO: |
| CAT | 391 | CACAGATGTAAATGTCTCGGAGAGGCT | 392 |
| | | CGGCCTGCCTACTGCCTCGGA | |
| CCCAAACC | SEQ ID | TGCTGTTGACAGTGAGCGCGCCCAAAC | SEQ ID |
| CACCAGTT | NO: | CCACCAGTTTAAATAGTGAAG | NO: |
| TAA | 393 | CCACAGATGTATTTAAACTGGTGGGTTT | 394 |
| | | GGGCATGCCTACTGCCTCGGA | |
| GCTGGTGA | SEQ ID | TGCTGTTGACAGTGAGCGAGGCTGGTG | SEQ ID |
| CCCATCTG | NO: | ACCCATCTGCAATTAGTGAAGC | NO: |
| CAA | 395 | CACAGATGTAATTGCAGATGGGTCACC | 396 |
| | | AGCCCTGCCTACTGCCTCGGA | |
| CTGACGAA | SEQ ID | TGCTGTTGACAGTGAGCGAGCTGACGA | SEQ ID |
| GCCCGAGA | NO: | AGCCCGAGATGTTTAGTGAAG | NO: |
| TGT | 397 | CCACAGATGTAAACATCTCGGGCTTCGT | 398 |
| | | CAGCGTGCCTACTGCCTCGGA | |

TABLE 3

| Mature | | | |
|---|---|---|---|
| product | | Hairpin* | |
| CCCATATC | SEQ ID | TGCTGTTGACAGTGAGCGAGCCCATATC | SEQ ID |
| ACCCAAGA | NO: | ACCCAAGAAGTTTAGTGAAG | NO: |
| AGT | 399 | CCACAGATGTAAACTTCTTGGGTGATAT | 400 |
| | | GGGCGTGCCTACTGCCTCGGA | |
| GCTTCCAG | SEQ ID | TGCTGTTGACAGTGAGCGAGGCTTCCA | SEQ ID |
| AAGAGCAT | NO: | GAAGAGCATCTTATAGTGAAG | NO: |
| CTT | 401 | CCACAGATGTATAAGATGCTCTTCTGGA | 402 |
| | | AGCCCTGCCTACTGCCTCGGA | |
| CTGTATCG | SEQ ID | TGCTGTTGACAGTGAGCGCGCTGTATCG | SEQ ID |
| GAATTGCG | NO: | GAATTGCGTTTATAGTGAAGC | NO: |
| TTT | 403 | CACAGATGTATAAACGCAATTCCGATA | 404 |
| | | CAGCATGCCTACTGCCTCGGA | |
| CTTATGAG | SEQ ID | TGCTGTTGACAGTGAGCGCGCTTATGA | SEQ ID |
| GATGGAAG | NO: | GGATGGAAGGAATTAGTGAAG | NO: |
| GAA | 405 | CCACAGATGTAATTCCTTCCATCCTCAT | 406 |
| | | AAGCTTGCCTACTGCCTCGGA | |
| CTGCTCTC | SEQ ID | TGCTGTTGACAGTGAGCGCGCTGCTTTC | SEQ ID |
| CAGGACAG | NO: | CAGGACAGGCAATAGTGAAG | NO: |
| GCA | 407 | CCACAGATGTATTGCCTGTCCTGGAAAG | 408 |
| | | CAGCTTGCCTACTGCCTCGGA | |
| CCGCCCAT | SEQ ID | TGCTGTTGACAGTGAGCGCGCCGCCCAT | SEQ ID |
| ATCACCCA | NO: | ATCACCCAAGAATAGTGAAG | NO: |
| AGA | 409 | CCACAGATGTATTCTTGGGTGATATGGG | 410 |
| | | CGGCTTGCCTACTGCCTCGGA | |
| GTGCGTGC | SEQ ID | TGCTGTTGACAGTGAGCGCCGTGCGTG | SEQ ID |
| CAGCGAGT | NO: | CCAGCGAGTCCATTAGTGAAGC | NO: |
| CCA | 411 | CACAGATGTAATGGACTCGCTGGCACG | 412 |
| | | CACGATGCCTACTGCCTCGGA | |
| CTCTGCGT | SEQ ID | TGCTGTTGACAGTGAGCGAGCTCTGCGT | SEQ ID |
| GCTGTATC | NO: | GCTGTATCGGAATAGTGAAGC | NO: |
| GGA | 413 | CACAGATGTATTCCGATACAGCACGCA | 414 |
| | | GAGCGTGCCTACTGCCTCGGA | |
| CTCATTAA | SEQ ID | TGCTGTTGACAGTGAGCGCGCTCATTAA | SEQ ID |
| GTCACAGA | NO: | GTCACAGAAATTTAGTGAAG | NO: |
| AAT | 415 | CCACAGATGTAAATTTCTGTGACTTAAT | 416 |
| | | GAGCTTGCCTACTGCCTCGGA | |
| CGAGTCCT | SEQ ID | TGCTGTTGACAGTGAGCGCCCGAGTCCT | SEQ ID |
| CTGGAAGT | NO: | CTGGAAGTCTTATAGTGAAGC | NO: |
| CTT | 417 | CACAGATGTATAAGACTTCCAGAGGAC | 418 |
| | | TCGGTTGCCTACTGCCTCGGA | |
| GAGTCCAT | SEQ ID | TGCTGTTGACAGTGAGCGACGAGTCCA | SEQ ID |
| CTCCCGGG | NO: | TCTCCCGGGCATATAGTGAAG | NO: |
| CAT | 419 | CCACAGATGTATATGCCCGGGAGATGG | 420 |
| | | ACTCGCTGCCTACCTGCCTCGGA | |
| CCGAGCCT | SEQ ID | TGCTGTTGACAGTGAGCGAGCCGAGCC | SEQ ID |
| CTCCGAGA | NO: | TCTCCGAGACATTTAGTGAAG | NO: |
| CAT | 421 | CCACAGATGTAAATGTCTCGGAGAGGC | 422 |
| | | TCGGCCTGCCTACTGCCTCGGA | |
| CCCAAACC | SEQ ID | TGCTGTTGACAGTGAGCGCGCCCAAAC | SEQ ID |
| CACCAGTT | NO: | CCACCAGTTTAAATAGTGAAG | NO: |
| TAA | 423 | CCACAGATGTATTTAAACTGGTGGGTTT | 424 |
| | | GGGCATGCCTACTGCCTCGGA | |
| GCTGGTGA | SEQ ID | TGCTGTTGACAGTGAGCGAGGCTGGTG | SEQ ID |
| CCCATCTG | NO: | ACCCATCTGCAATTAGTGAAG | NO: |
| CAA | 425 | CCACAGATGTAATTGCAGATGGGTCAC | 426 |
| | | CAGCCCTGCCTACTGCCTCGGA | |
| CTGACGAA | SEQ ID | TGCTGTTGACAGTGAGCGAGCTGACGA | SEQ ID |
| GCCCGAGA | NO: | AGCCCGAGATGTTTAGTGAAG | NO: |
| TGT | 427 | CCACAGATGTAAACATCTCGGGCTTCGT | 428 |
| | | CAGCGTGCCTACTGCCTCGGA | |

*shRNA sequences from Cold Spring Harbor RNAi Codex (//codex.cshl.edu/scripts/newmain.pl)

### Therapeutic Indications

As demonstrated herein, SHIP inhibitors, e.g., a SHIP1 siRNA, may be used to reduce the expression or activity of SHIP in hematopoietic cells. In addition, SHIP inhibitors may be used to reduce or prevent apoptosis of hematopoetic cells, including hematopoietic progenitor cells in particular. Such apoptosis may be naturally-occurring apoptosis or apoptosis induced by an agent or environmental stress, such as treatment with a chemotherapeutic agent or radiation. SHIP inhibitors may also be used to enhance proliferation of hematopoietic cells, including hematopoetic progenitor cells in particular.

SHIP inhibitors may be used to treat myelosuppression, e.g., immune suppression. In some embodiments, SHIP inhibitors may be used to accelerate or increase peripheral blood cell numbers after hemodepletion, for example, after chemotherapy or radiotherapy of solid tumours, or in any situation resulting in depletion of hemopoietic cells. In particular embodiments of the present invention, SHIP1-specific inhibitors are used to protect hematopoietic cells from cell death or increase their proliferation, e.g., before, during, or following treatment with one or more agents capable of inducing myelosuppression. Such SHIP1-specific inhibitors are advantageous as compared to drugs currently used to expand hematopoietic cells following chemotherapy, since SHIP1-specific inhibitors are pan-hematopoietic cell specific, while most currently used drugs act on only a subset or particular type of hematopoietic cell. By “hemodepletion” is meant a decrease in hematopoietic cells, including white blood cells, red blood cells, and platelets.

In alternative embodiments, SHIP inhibitors may be used, for example, in combination with erythropoietin (EPO) to reverse the anemia that is associated with advanced solid cancers or to increase neutrophils during a systemic infection. In alternative embodiments, SHIP inhibitors may be used to protect hemopoietic cells such as progenitors and mature blood cells, for example, before or during solid tumour chemotherapy and radiotherapy. Thus, in various embodiments, a SHIP inhibitor may be provided to a patient before, during, or after (or any combination thereof) treatment with a chemotherapeutic agent and/or radiotherapy.

In one embodiment, a SHIP1 inhibitor is used in combination with one or more chemotherapeutic agents and/or radiation to treat a solid tumor. The SHIP1 inhibitor protects the hematopoietic cells from killing by the chemotherapeutic agent(s) and/or radiation, thereby allowing the patient to be treated with an increased total amount or higher dosage of the chemotherapeutic agent(s) and/or radiation. For example, one or more chemotherapeutic agents and/or radiation may be administered to the patient in an amount or dosage higher than those normally used or approved, when provided in combination with a SHIP inhibitor.

In a related embodiment, a SHIP inhibitor is provided to a patient in combination with another agent used to stimulate hematopoietic cell proliferation following chemotherapy, such as, e.g., granulocyte colony stimulating factor (G-CSF), granulocyte macrophage colony stimulating factor (GM-CSF), macrophage colony stimulating factor (M-CSF), interleukin 3, or thrombopoietin. In an alternative embodiment, a SHIP inhibitor is provided to a patient to expand hemopoietic cells, e.g., red blood cells, following dialysis.

Cancers include solid tumours and non-solid tumours. Solid tumours include carcinomas, which are the predominant cancers and are cancers of epithelial cells or cells covering the external or internal surfaces of organs, glands, or other body structures (e.g., skin, uterus, lung, breast, prostate, stomach, bowel), and which tend to metastasize; sarcomas, which are derived from connective or supportive tissue (e.g., bone, cartilage, tendons, ligaments, fat, muscle); Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). In addition, solid tumours include mixed type cancers, such as adenosquamous carcinomas, mixed mesodermal tumors, carcinosarcomas, or teratocarcinomas.

Hematologic tumours are derived from bone marrow and lymphatic tissue. Hematologic tumours may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythrernia (various blood cell products, but with red cells predominating); or lymphomas, which may be solid tumors and which develop in the glands or nodes of the lymphatic system, and which may be Hodgkin or Non-Hodgkin lymphomas. In some embodiments, hematologic tumours, such as leukemias or lymphomas (e.g., acute lymphoblastic leukemia, acute myeloblastic leukemia, chronic myelogenous leukemia, Hodgkin's disease, multiple myeloma, non-Hodgkin's lymphoma), are specifically excluded.

### Test Compounds

SHIP inhibitors according to the invention include, without limitation, molecules selective for SHIP, analogs and variants thereof, including, for example, the molecules described herein. SHIP inhibitors may be identified using a variety of techniques, including screening of combinatorial libraries or using predictive software. In general, test compounds are identified from large libraries of both natural products or synthetic (or semi-synthetic) extracts or chemical libraries according to methods known in the art. Those skilled in the field of drug discovery and development will understand that the precise source of test extracts or compounds is not critical to the method(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available. Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla., USA), and PharmaMar, MA, USA. Furthermore, if desired, any library or compound is readily modified using standard chemical, physical, or biochemical methods.

SHIP inhibitors may be identified based upon the ability of a test compound to inhibit SHIP expression or activity, using routine methods available in the art. Identified SHIP inhibitors may be subsequently evaluated for their ability to protect hematopoietic cells, e.g., from a chemotherapeutic agent or radiation. In one embodiment, when a crude extract is found to protect hemopoietic cells, further fractionation of the positive lead extract is necessary to isolate chemical constituents responsible for the observed effect. Thus, the goal of the extraction, fractionation, and purification process is the careful characterization and identification of a chemical entity within the crude extract having protective, e.g., myeloprotective, activities. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic, prophylactic, diagnostic, or other value may be subsequently analyzed using a SHIP knockout animal model, or any other animal model suitable for immune suppression or myelosuppression.

### Chemotherapeutic Agents

A “chemotherapeutic agent” or “chemotherapeutic” refers to a chemical compound or composition mat may be used to treat a disease in a patient. In alternative embodiments, chemotherapeutics include cancer chemotherapeutics. In alternative embodiments, chemotherapeutics include alkylating and oxidizing agents, antimetabolites, antibiotics, mitotic inhibitors, chromatin function inhibitors, hormone and hormone inhibitors, antibodies, immunomodulators, angiogenesis inhibitors, rescue/protective agents, etc.

Alkylating and oxidizing agents include nitrogen mustards, ethylenimines, alkyl sulfonates, nitrosureas, triazenes, platinum coordinating complexes, etc. Nitrogen mustards include mechlorethamine (Mustargen™), cyclophosphamide (Cytoxan™ and Neosar™), ifosfamide (Ifex™), phenylalanine mustard, melphalen (Alkeran™), chlorambucil (Leukeran™), uracil mustard and estramustine (Emcyt™); ethylenimines include thiotepa (Thioplex™); alkyl sulfonates include busulfan (Myerlan™); nitrosureas include lomustine (CeeNU™), carmustine (BiCNU™ and BCNU™) streptozocin (Zanosar™), etc.; triazines include dicarbazine (DTIC-Dome™), temozolamide (Temodar™), etc.; platinum coordination complexes include cis-platinum, cisplatin (Platinol™ and Platinol AQ™), carboplatin (Paraplatin™), etc. Other examples of alkylating and oxidizing agents include altretamine (Hexalen™) and arsenic (Trisenox™).

Antimetabolites include folic acid analogs, pyrimidine analogs and purine analogs. Folic acids include methotrexate (Amethopterin™, Folex™, Mexate™, Rheumatrex™), etc.; pyrimidine analogs include 5-fluoruracil (Adrucil™, Efudex™, Fluoroplex™), floxuridine, 5-fluorodeoxyuridine (FUDR™), capecitabine (Xeloda™), flurdarabine (Fludara™), cytosine arabinoside (Cytaribine™, Cyrosar™, ARA-C™), etc.; purine analogs include 6-mercaptopurine (Purinethol), 6-thioguanine (Thioguanine™), gemcitabine (Gemzar™), cladribine (Leustatin™), deoxycoformycin and pentostatin (Nipent™), etc.

Antibiotics include doxorubicin (Adriamycin™, Rubex™, Doxil™, Daunoxome™-liposomal preparation), daunorubicin (Daunomycin™, Cerubidine™), idarubicin (Idamycin™), valrubicin (Valstar™), epirubicin, mitoxantrone (Novantrone™), dactinomycin (Actinomycin D™, Cosmegen™), mithramycin, plicamycin (Mithracin™), mitomycin C (Mutamycin™), bleomycin (Blenoxane™), procarbazine (Matulane™), etc.

Mitotic inhibitors include taxanes or diterpenes and vinca alkaloids. Examples of taxanes include paclitaxel (Taxol™) and docetaxel (Taxotere™). Examples of vinca alkaloids include vinblastine sulfate (Velban™, Velsar™, VLB™), vincristine sulfate (Oncovin™, Vincasa PFS™, Vincrex™) and vinorelbine sulfate (Navelbine™).

Chromatin function inhibitors include camptothecins and epipodophyllotoxins. Examples of camptothecins include topotecan (Camptosar™) and irinotecan (Hycamtin™). Examples of epipodophyllotoxins include etoposide (VP-16™, VePesid™ and Toposar™) and teniposide (VM-26™ and Vumon™).

Hormone and hormone inhibitors include estrogens, antiestrogens, aromatase inhibitors, progestins, GnRH agonists, androgens, antiandrogens and inhibitors of syntheses. Examples of estrogens include diethylstilbesterol (Stilbesterol™ and Stilphostrol™), estradiol, estrogen, esterified estrogens (Estratab™ and Menest™) and estramustine (Emcyt™). Examples of anti-estrogens include tamoxifin (Nolvadex™) and torernifene (Fareston™). Examples of aromatase inhibitors include anastrozole (Arimidex™) and letrozol (Femara™). Examples of progestins include 17-OH-progesterone, medroxyprogesterone, and megastrol acetate (Megace™). Examples of GnRH agonists include gosereline (Zoladex™) and leuprolide (Leupron™). Examples of androgens include testosterone, methyltestosterone and fluoxmesterone (Android-F™, Halotestin™). Examples of antiandrogens include flutamide (Eulexin™), bicalutamide (Casodex™) and nilutamide (Nilandron™). Examples of inhibitors of synthesis include aminoglutethimide (Cytadren™) and ketoconozole (Nizoral™).

Antibodies include rituximab (Rituxan™), trastuzumab (Herceptin™), gemtuzumab ozogamicin (Mylotarg™), tositumomab (Bexxar™) and bevacizumab. These chemotherapeutics may be antibodies that are targeted to a particular protein on the cell surface of a cancer cell. These antibodies may provide a motif for generating an immune response to the antibody and hence the cancer cell or possibly induce apoptosis. Other mechanisms of action of this class of chemotherapeutic include inhibiting stimulation from growth factors by binding to receptors on cancer cells.

Immunomodulators include denileukin diftitox (Ontak™), levamisole (Ergamisol™), bacillus Calmette-Gueran, BCG (TheraCys™, TICE BCG™), interferon alpha-2a, interferon alpha-2b (Roferon-A™, Intron A™) and interleukin-2 and aldesleukin (ProLeukin™).

Angiogenesis inhibitors include thalidomide (Thalomid™), angiostatin and endostatin. Rescue/protective agents include dexrazoxane (Zinecard™), amifostine (Ethyol™), G-CSF (Neupogen™), GM-CSF (Leukine™), erythopoetin (Epogen™, Procrit™), oprelvekin and IL-11 (Neumega™). Other cancer chemotherapeutics include imatinib mesylate, STI-571 (Gleevec™), 1-aspariginase (Elspar™, Kidrolase™), pegaspasgase (Oncaspar™), hydroxyurea (Hydrea™, Doxia™), leucovorin (Wellcovorin™), mitotane (Lysodren™), porfimer (Photofrin™), tretinoin (Veasnoid™), oxaliplatin, etc.

In alternative embodiments, compositions according to the invention may be administered in combination with radiotherapy or a chemotherapeutic agent, such as a cancer therapeutic, as described herein or known in the art. In alternative embodiments, the chemotherapeutic is known to induce immune suppression or myelosuppression. In alternative embodiments, the chemotherapeutic is suspected of causing, or belongs to a class of compounds that induce, immune suppression or myelosuppression.

### Pharmaceutical Compositions and Administration

SHIP inhibitors may be provided alone or in combination with other compounds (for example, chemotherapeutics), in the presence of a liposome, an adjuvant, or any pharmaceutically acceptable carrier, in a form suitable for administration to mammals, for example, humans, cattle, sheep, etc. If desired, treatment with a compound according to the invention may be combined with more traditional and existing therapies for immune suppression or myelosuppression. SHIP inhibitors may also be provided in combination with radiotherapy.

SHIP inhibitors may be provided chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature. In alternative embodiments, SHIP inhibitors are administered to a subject in need of such inhibitors, e.g., a subject undergoing a chemotherapy or a radiotherapy, or any therapy likely to cause depletion of hemopoietic cells, such as HPCs. In alternative embodiments, SHIP inhibitors may be administered to a subject for short periods of time e.g, 1 or 2 days, or up to 48 hours, or for sufficient time to protect HPCs. In alternative embodiments, SHIP inhibitors may be administered to a subject before or during a chemotherapy or a radiotherapy, or any therapy likely to cause depletion of hemopoietic cells, such as HPCs. In alternative embodiments, SHIP inhibitors may be administered to a subject after a chemotherapy or a radiotherapy, or any therapy likely to cause depletion of hemopoietic cells.

In alternative embodiments, a SHIP inhibitor, e.g., a siRNA selective for SHIP1, may be effectively delivered to hemopoietic cells by a variety of methods known to those skilled in the art. Such methods include but are not limited to liposomal encapsulation/delivery, vector-based gene transfer, fusion to peptide or immunoglobulin sequences for enhanced cell targeting and other techniques.

In alternative embodiments, a SHIP inhibitor, e.g., an siRNA selective for SHIP1, may also be formulated in pharmaceutical compositions well known to those in the field. These include liposomal formulations and combinations with other agents or vehicles/excipients such as cyclodextrins which may enhance delivery of the active siRNA. In alternative embodiments, suitable carriers include lipid-based carriers such as a stabilized nucleic acid-lipid particle (e.g., SNALP or SPLP), cationic lipid or liposome nucleic acid complexes (i.e., lipoplexes), a liposome, a micelle, a virosome, or a mixture thereof. In other embodiments, the carrier system is a polymer-based carrier system such as a cationic polymer-nucleic acid complex (i.e., polyplex). In alternative embodiments, the carrier system is a cyclodextrin-based carrier system such as a cyclodextrin polymer-nucleic acid complex. In further embodiments, the carrier system is a protein-based carrier system such as a cationic peptide-nucleic acid complex.

Suitable carriers are known in the art and are described in, without limitation, is United States Patent Application Nos. 20070173476 published Jul. 26, 2007; 20050008617 published Jan. 13, 2005; 20050014962 published Jan. 20, 2005; 20050064595 published Mar. 24, 2005; 20060008910 published Jan. 12, 2006; 20060051405 published Mar. 9, 2006; 20060083780 published Apr. 20, 2006; 20050008689 published Jan. 13, 2005; 20070172950 published Jul. 26, 2007; U.S. Pat. No. 7,101,995 issued Sep. 5, 2006 to Lewis, et al.; U.S. Pat. No. 7,220,400 issued May 22, 2007, to Monahan, et al.; U.S. Pat. No. 5,705,385 issued Jan. 6, 1998 to Bally, et al.; U.S. Pat. No. 5,965,542 issued Oct. 12, 1999 to Wasan, et al.; U.S. Pat. No. 6,287,591 issued Sep. 11, 2001 to Semple, et al., all of which are hereby incorporated by reference.

In one embodiment, the present invention contemplates a nucleic acid-lipid particle comprising a nucleic acid inhibitor of a SHIP, such as an siRNA specific for a SHIP, e.g., SHIP1. In addition to the references described above, suitable nucleic acid-lipid particles and their use are described in U.S. Pat. Nos. 6,815,432, 6,586,410, and 6,534,484. In particular embodiments, the nucleic acid-lipid particle comprises a nucleic acid inhibitor of SHIP, a cationic lipid, and a modified lipid that prevents aggregation of particles. The particle may further comprise a non-cationic lipid. In particular embodiments, the nucleic acid inhibitor of SHIP is an antisense oligonucleotide, an siRNA, or a miRNA that specifically targets a SHIP polynucleotide.

Conventional pharmaceutical practice may be employed to provide suitable formulations or compositions to administer the compounds to subjects suffering from, at risk of, or presymptomatic for immune suppression or myelosuppression. Suitable pharmaceutical compositions may be formulated by means known in the art and their mode of administration and dose determined by the skilled practitioner. Any appropriate route of administration may be employed, for example, parenteral, intravenous, subcutaneous, intramuscular, intracranial, intraorbital, ophthalmic, intraventricular, intracapsular, intraspinal, intrathecal, intracisternal, intraperitoneal, intranasal, aerosol, lavage, topical, oral administration, or any mode suitable for the selected treatment. Therapeutic formulations may be in the form of liquid solutions or suspensions. For enteral administration, the compound may be administered in a tablet, capsule or dissolved in liquid form. The table or capsule may be enteric coated, or in a formulation for sustained release. For intranasal formulations, in the form of powders, nasal drops, or aerosols. For parenteral administration, a compound may be dissolved in sterile water or saline or a pharmaceutically acceptable vehicle used for administration of non-water soluble compounds such as those used for vitamin K.

Methods well known in the art for making formulations are found in, for example, Remington: the Science & Practice of Pharmacy by Alfonso Gennaro, 20^{th} ed., Williams & Wilkins, (2000). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. For therapeutic or prophylactic compositions, the compounds are administered to an individual in an amount sufficient to stop or slow hemopoietic cell death, or to enhance the proliferation of hemopoietic cells.

An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as treatment of immune suppression or myelosuppression. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as prevention or protection against hemopoietic cell death or maintenance of hemopoietic cells. Typically, a prophylactic dose is used in subjects prior to or at an earlier stage of disease, so that a prophylactically effective amount may be less than a therapeutically effective amount. A preferred range for therapeutically or prophylactically effective amounts of a compound may be any integer from 0.1 nM-0.1M, 0.1 nM-0.05M, 0.05 nM-15 μM or 0.01 nM-10 μM.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

As used herein, a subject may be a human, non-human primate, rat, mouse, cow, horse, pig, sheep, goat, dog, cat, etc. The subject may be a clinical patient, a clinical trial volunteer, an experimental animal, etc. The subject may be suspected of having or at risk for immune suppression or myelosuppression, be diagnosed with immune suppression or myelosuppression, or be a control subject that is confirmed to not have immune suppression or myelosuppression. Diagnostic methods for immune suppression or myelosuppression and the clinical delineation of immune suppression or myelosuppression diagnoses are known to those of ordinary skill in the art.

The present invention will be further illustrated in the following examples.

### Example 1

### siRNA Mediated Knock-Down of SHIP Expression Enhances PIP3Dependent Signaling

Small interfering (si)RNAs were demonstrated to markedly reduce SHIP levels when transfected into the human erythroleukemic cell line, TF1, or the mouse cell line, EL-4. More specifically, various siRNAs selective for mouse and human SHIP 1 sequences were tested.

The following siRNAs (with their position relative to the target sequence indicated) were directed against the sequence described in GenBank Accession No. U51742, which describes mouse SHIP mRNA:

| | |
|---|---|
| SHIP1(sSHIP): | |
| CCC ACT AGT TGT TGA ACT TTA | (SEQ ID NO: 5) |
| SHIP2(2080): | |
| AAC AGG GAT GAA GTA CAA CTT | (SEQ ID No: 6) |
| SHIP3(1509): | |
| AAG TCA CCA GCA TGA CAT TTA | (SEQ ID NO: 7) |
| SHIP4(2991): | |
| AAC CAC CTC TGT CGC CAA AGA | (SEQ ID NO: 8) |
| SHIP2a(AS/188): | |
| ATG GAC TCG CTG GCA CGC AC | (SEQ ID NO: 9) |
| SHIP1a(2381): | |
| AAG AGT CAG GAA GGA GAG AAT | (SEQ ID NO: 10) |

The following siRNAs (with their position relative to the target sequence indicated) were directed against the sequence described in GenBank Accession No. NM_{—}001017915, which describes human SHIP mRNA:

| | |
|---|---|
| C) 2437-AAGAGTCAGGAAGGAGAAAAT | (SEQ ID NO: 11) |
| B) 1749-AACCTCCTTAGGGTTCGTCAA | (SEQ ID NO: 12) |
| A) 359-AAGGCGTCTCCATGAGGTTCT | (SEQ ID NO: 13) |
| D) 2728-AAGACGAGGGAGAAGCTCTAT | (SEQ ID NO: 14) |

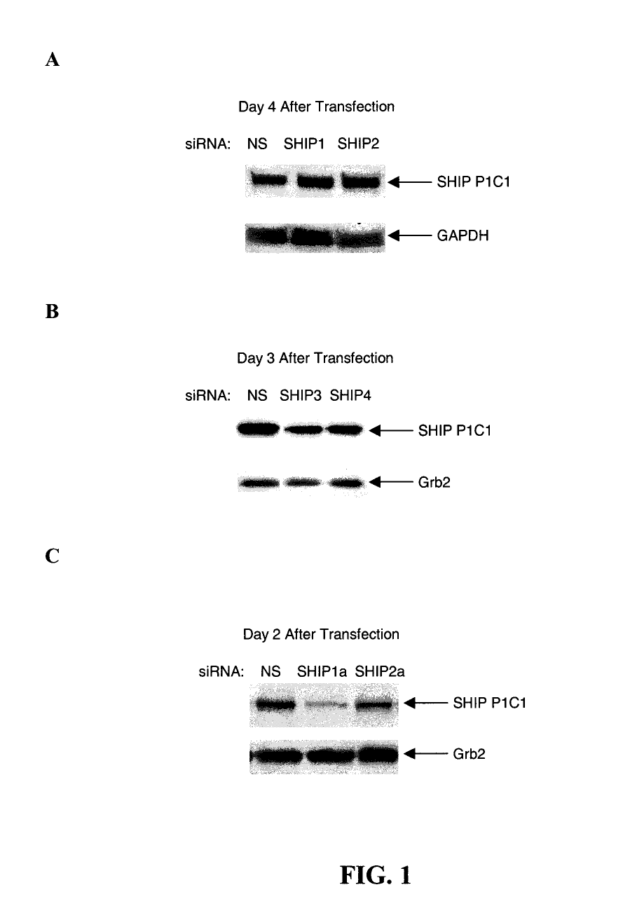

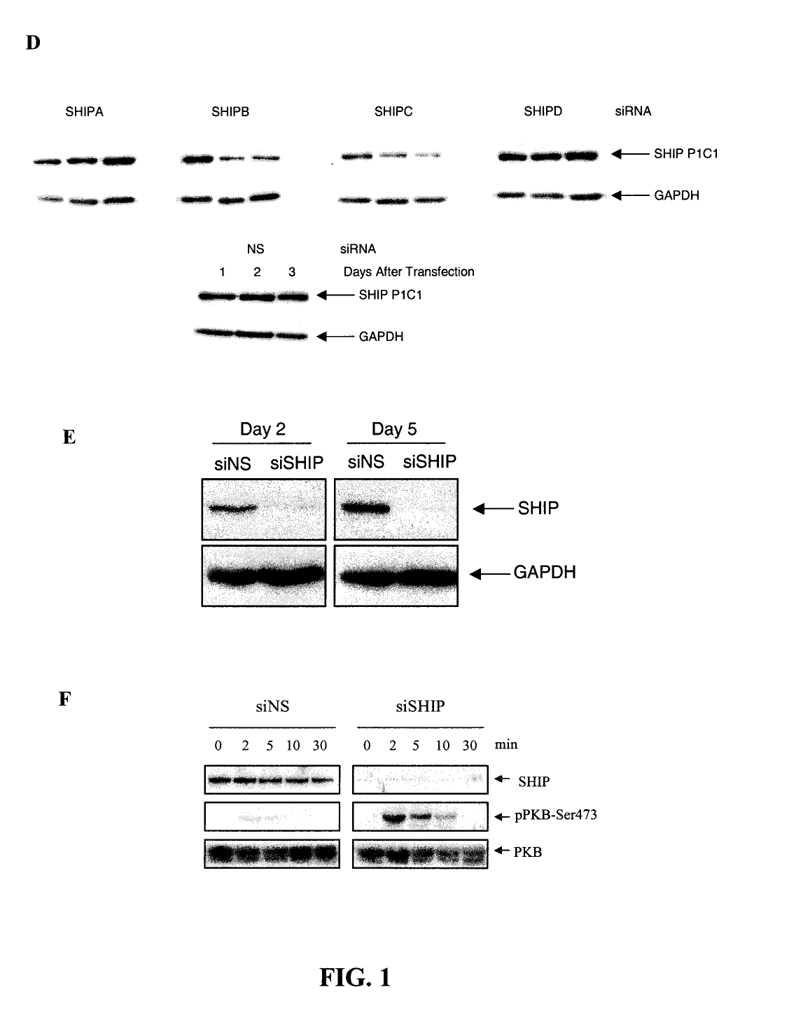

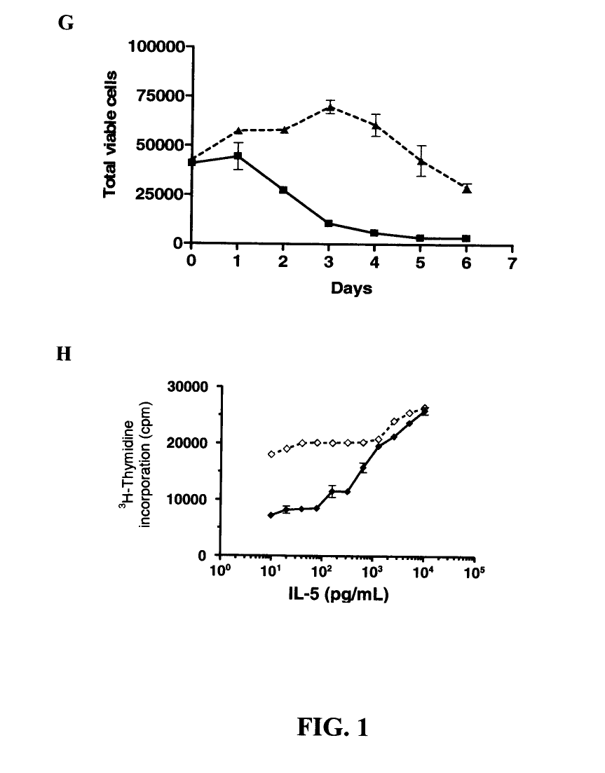

EL-4 (mouse) or TF1 (human) hemopoietic progenitor lines were transduced with the indicated siRNAs to SHIP1 or a control non-silencing siRNA (NS or siNS). Cell lysates were prepared on the indicated days and assessed for SHIP1 and control GAPDH protein expression by immunoblot analyses (FIGS. 1A-C, siRNA to mouse SHIP1 in EL-4 cells; FIGS. 1D-E, siRNA to human SHIP1 in TF-1 cells).

TF1 cells transfected with siSHIP (AAGAGTCAGGAAGGAGAAAAT, SEQ ID NO: 11) or siNS were stimulated with the cytokine GM-CSF for the indicated length of time. Cell lysates were prepared and subjected to immunoblot analysis with antibodies against SHIP, the PIP3 dependent kinase PKB or phospho PKB (Ser 473) (FIG. 1B), siRNAs effectively reduced SHIP1 levels, as assessed by both Western analysis (FIGS. 1A-E). Inhibition of SHIP1 expression enhanced the activation of the PIP3 dependent kinase PKB (FIG. 1F).

### Example 2

### siRNA Mediated Inhibition of SHIP1 Expression Enhances Cell Survival and Proliferation

TF1 cells transfected with siSHIP (triangles) or siNS (squares) were cultured in the absence of growth factors and the total number of viable cells counted daily by trypan blue exclusion (FIG. 1G), TF1 cells were cultured in the presence of increasing concentrations of the growth promoting cytokine IL-5, 2 days after siRNA transfection. Proliferation of siSHIP (diamonds) and control siNS (solid diamonds) transfected TF-1 cells was measured by [³H]-thymidine incorporation (FIG. 1H). Inhibition of SHIP expression considerably increased survival of these cells (FIG. 1G) and proliferation in response to sub-optimal levels of IL-5 (FIG. 1H).

### Example 3

### siRNA-Mediated Knock-Down of SHIP 1 Expression Enhances Resistance to Chemotherapy Drugs

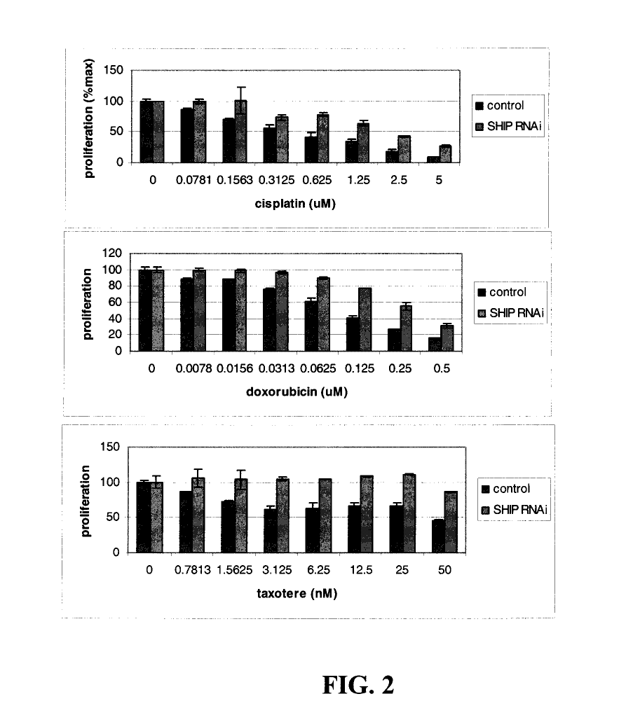

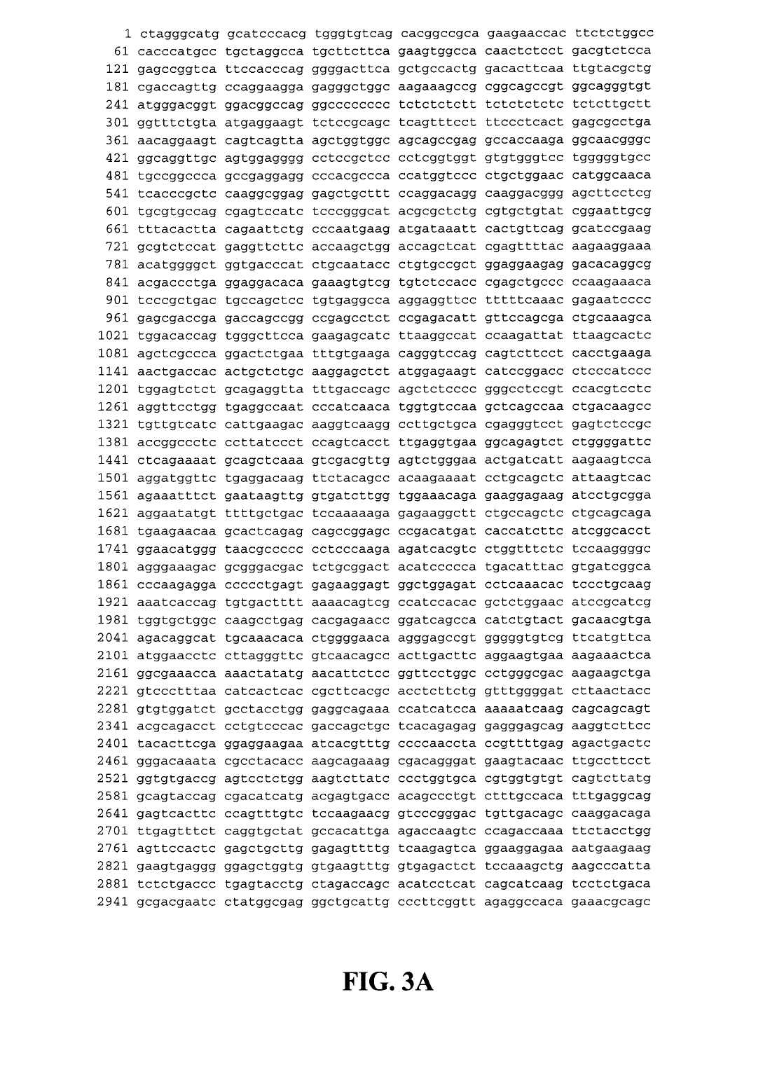

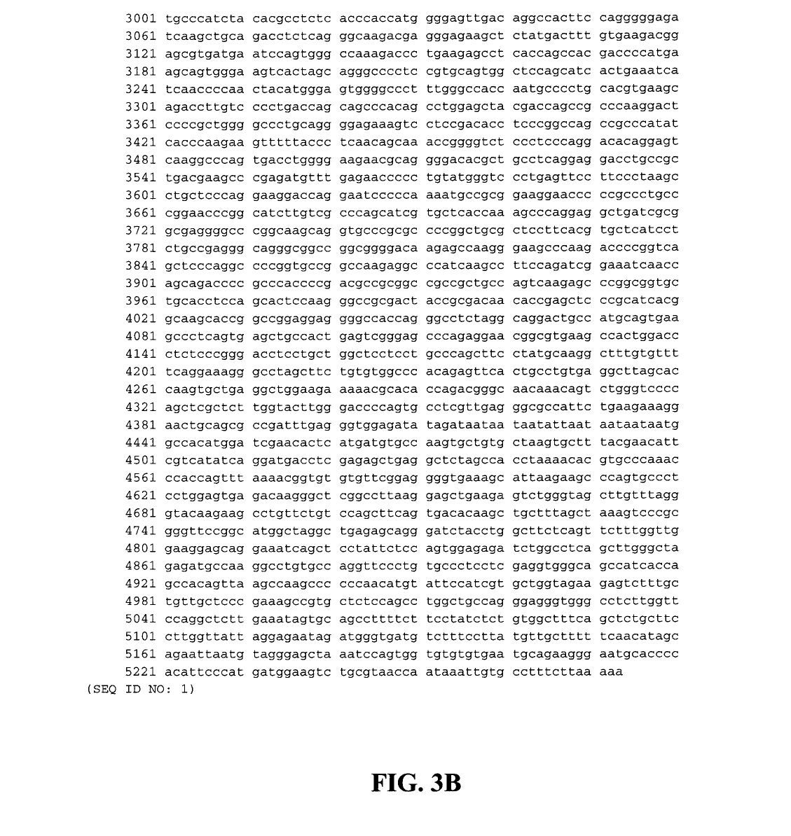

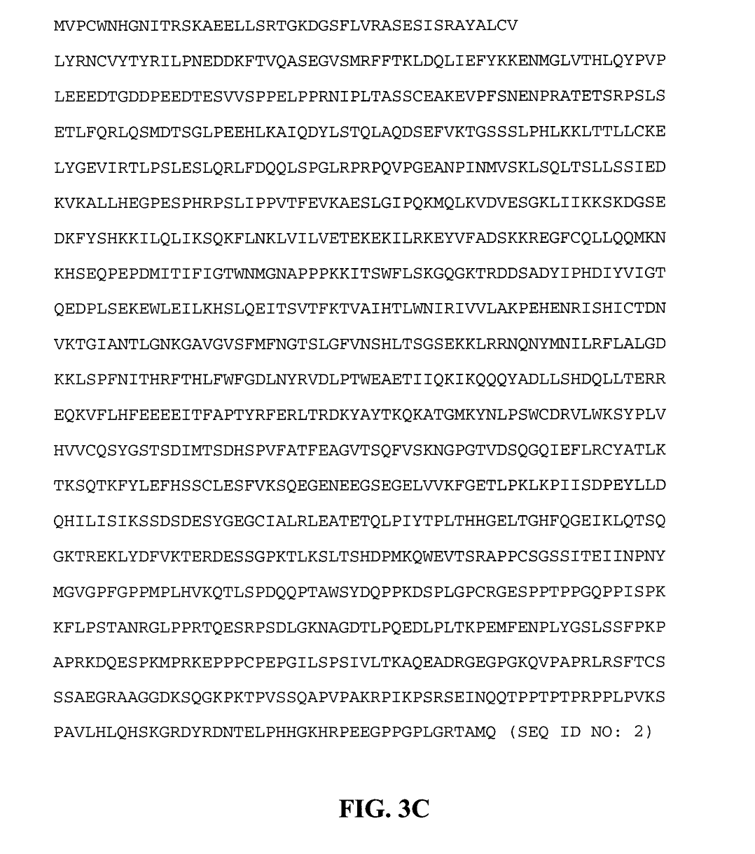

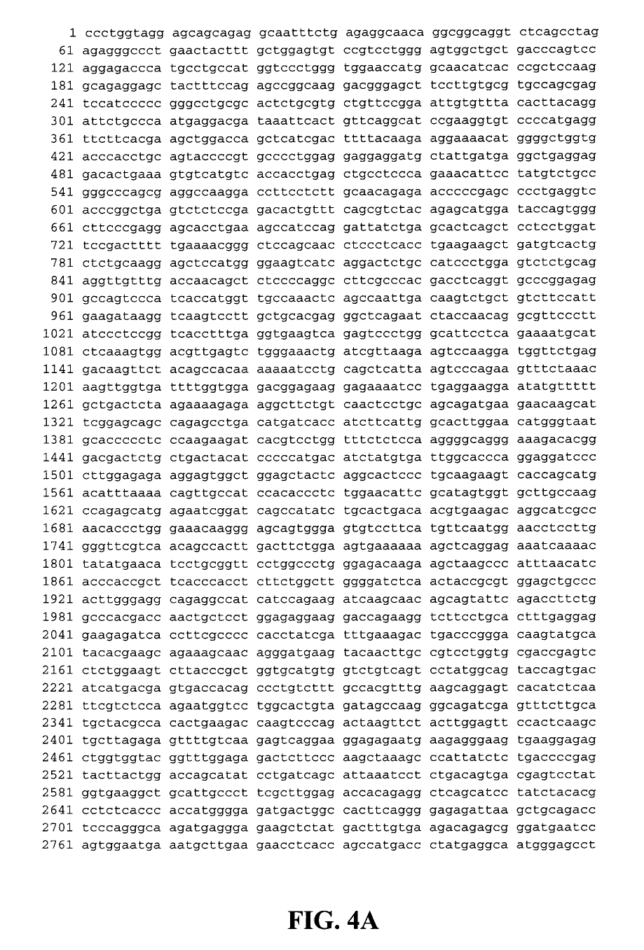

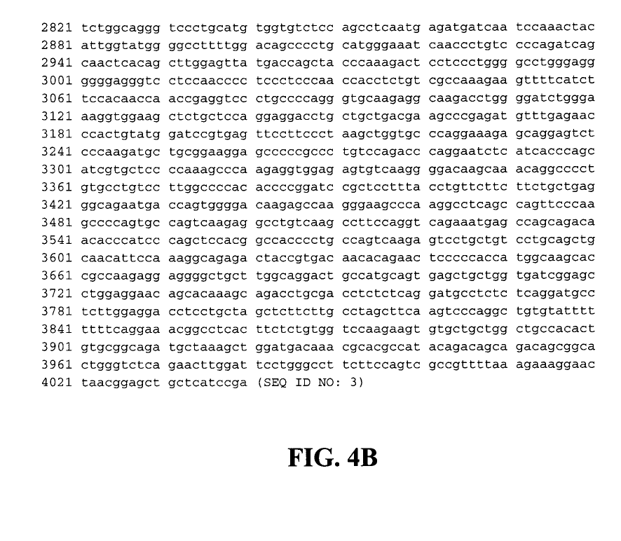

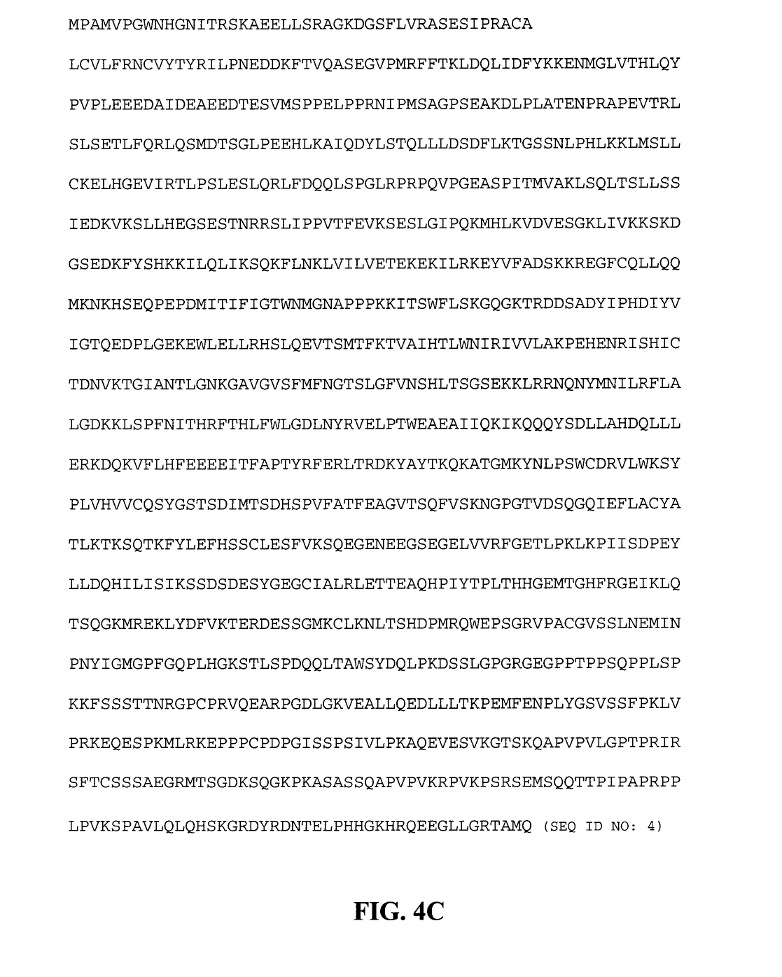

The TF1 hemopoietic progenitor cell line was transfected with SHIP1 siRNA or control siRNA as in FIG. 1. After 4 days, the cells were assessed at the indicated concentrations of cisplatin, doxorubicin and taxotere in the presence of complete growth media, [³H]-thymidine incorporation was measured 2 days later. The results indicate that TF1 cells in which SHIP1 is silenced are significantly more resistant to three common chemotherapy drugs used to treat solid tumours (FIG. 2).

### REFERENCES

1. Sly L M, Rauh M J, Kalesnikoff J, Buchse T, Krystal G. SHIP, SHIP2 and PTEN activities are regulated in vivo by modulation of their protein levels; SHIP is upregulated in macrophages and mast cells by lipopolysaccharide. Exp Hematol. 2003; 31:1170-1181.
2. Helgason C D, Damen J E, Rosten P, et al. Targeted disruption of SHIP leads to hemopoietic perturbations, lung pathology, and a shortened life span. Genes Dev. 1998; 12:1610-1620.
3. Liu L, Damen J E, Huber M, et al. SHIP accelerates ceramide induced apoptosis in hemopoietic cell lines [abstract]. Blood, 1 997; 90,307a.
4. Liu Q, Sasaki T5 Kozieradzki I, et al. SHIP is a negative regulator of growth factor receptor-mediated PKB/Akt activation and myeloid cell survival. Genes Dev. 1999; 13:786-791.
5. Wang H, Li M, Rinehart J J, Zhang R, Dexamethasone as a chemoprotectant in cancer chemotherapy: hematoprotective effects and altered pharmacokinetics and tissue distribution of carboplatin and gemcitabine. Cancer Chemother Pharmacol. 2004; 53:459-467.
6. Sorrentino B P. Gene therapy to protect haematopoietic cells from cytotoxic cancer drugs. Nat Rev Cancer. 2002; 2:431-441.
7. Rosenfeld C S, Nemunaitis J. The role of granulocyte-macrophage colony-stimulating factor-stimulated progenitor cells in oncology. Semin Hematol. 1992; 29:19-26,
8. Bronchud M H, Howell A, Crowther D, Hopwood P, Souza L, Dexter T M. The use of granulocyte colony-stimulating factor to increase the intensity of treatment with doxorubicin in patients with advanced breast and ovarian cancer. Br J Cancer. 1989; 60:121-125.
9. Armitage J O. Emerging applications of recombinant human granulocyte-macrophage colony-stimulating factor. Blood. 1 998; 92:4491-4508,
10. Armstrong D K, Davidson N E. Dose intensity for breast cancer. Oncology (Williston Park), 2001; 15:701-8, 712.
11. Rameh L E, Cantley L C. The role of phosphoinositide 3-kinase lipid products in cell function, J Biol Chem. 1999; 274:8347-8350.
12. Maxwell M J, Yuan Y, Anderson K E, Hibbs M L, Salem H H, Jackson S P. SHIP1 and lyn kinase negatively regulate integrin aIIbb3 signalling in platelets, J Biol Chem. 2004; 279:32196-32204.
13. Riesterer O, Tenzer A, Zingg D, et al. Novel radiosensitizers for locally advanced epithelial tumors: inhibition of the PI3K/Akt survival pathway in tumor cells and in tumor-associated endothelial cells as a novel treatment strategy? Int J Radiat Oncol Biol Phys. 2004; 58:361-368.
14. Kim L A, Bae S S, Fernandes A, et al. Selective inhibition of Ras, phosphoinositide 3 kinase, and Akt isoforms increases the radiosensitivity of human carcinoma cell lines. Cancer Res. 2005; 65:7902-7910.
15. Coffey J C, Wang J H, Smith M J, et al. Phosphoinositide 3-kinase accelerates postoperative tumor growth by inhibiting apoptosis and enhancing resistance to chemotherapy-induced apoptosis. Novel role for an old enemy. J Biol Chem. 2005; 280:20968-20977.
16. Tu Z, Ninos J M, Ma Z, et al. Embryonic and hematopoietic stem cells express a novel SH2-containing inositol 5′-phosphatase isoform that partners with the Grb2 adapter protein. Blood. 2001; 98:2028-2038.
17. Kim C H, Hangoc G, Cooper S, et al. Altered responsiveness to chemokines due to targeted disruption of SHIP. J Clin Invest. 1999; 104:1751-1759,
18. Sly L M, Rauh M J, Kalesnikoff J, Song C H, Krystal G, LPS-induced upregulation of SHIP is essential for endotoxin tolerance. Immunity, 2004; 21.227-239.
19. Gardai S, Whitlock B B, Helgason C, et al. Activation of SHIP by NADPH oxidasestimulated Lyn leads to enhanced apoptosis in neutrophils. J Biol Chem. 2002; 277:5236-5246.
20. Cox D, Dale B M, Kashiwada M, Helgason C, Greenberg S. A regulatory role for Src homology 2 domain-containing inositol 5′-phosphatase (SHIP) in phagocytosis mediated by Fcg receptors and complement receptor 3 (aMb2; CD11 b/CD1 8). J Exp Med. 2001; 193:61-71.
21. Mason J M, Halupa A, Hyam D, Iscove N N, Dumont D J, Barber D L. SHIP1 regulates the proliferation and mobilization of the erythroid lineage [abstract]. Blood. 2002; 100, 51 9a

All citations are hereby incorporated by reference.

The present invention has been described with regard to one or more embodiments. However, it will be apparent to persons skilled in the art that a number of variations and modifications can be made without departing from the scope of the invention as defined in the claims.

1-45. (canceled)

46. A method of protecting a hemopoietic cell in a subject in need thereof, the method comprising administering an effective amount of an inhibitor of a hemopoietic-restricted SH2-containing inositol-5′-phosphatase to said subject.

47. The method of claim 46, wherein the hemopoietic-restricted SH2-containing inositol-5′-phosphatase is a SHIP1 molecule.

48. The method of claim 46, wherein the protecting comprises decreasing cell death.

49. The method of claim 46, wherein the subject has, or is suspected of having, a cancer.

50. The method of claim 46, wherein the subject is undergoing chemotherapy or radiotherapy.

51. The method of claim 46, further comprising administering a chemotherapeutic agent or administering a radiotherapy to said subject.

52. The method of claim 46, wherein the inhibitor is a siRNA or a small molecule.

53. The method of claim 52, wherein the siRNA comprises a sense strand consisting essentially of the sequence AAGAGTCAGGAAGGAGAGAAT (SEQ ID NO:10) or AAGAGTCAGGAAGGAGAAAAT (SEQ ID NO:11) or the complement thereof.

54. A method of treating or preventing myelosuppression in a subject in need thereof, comprising administering an effective amount of an inhibitor of a hemopoietic-restricted SH2-containing inositol-5′-phosphatase to said subject.

55. The method of claim 54, wherein the hemopoietic-restricted SH2-containing inositol-5′-phosphatase is a SHIP1 molecule.

56. The method of claim 54, wherein the myelosuppression comprises immune suppression.

57. The method of claim 54, wherein the myelosuppression is induced by chemotherapy or by radiotherapy.

58. The method of claim 54, wherein the treating comprises increasing proliferation or reducing death of a hemopoietic cell.

59. The method of claim 54, wherein the inhibitor is a siRNA or a small molecule.

60. The method of claim 59, wherein the siRNA comprises a sense strand consisting essentially of the sequence AAGAGTCAGGAAGGAGAGAAT (SEQ ID NO:10) or AAGAGTCAGGAAGGAGAAAAT (SEQ ID NO:11) or the complement thereof.

61. A siRNA molecule comprising a sense strand consisting essentially of the sequence AAGAGTCAGGAAGGAGAGAAT (SEQ ID NO:10) or AAGAGTCAGGAAGGAGAAAAT (SEQ ID NO:11) or the complement thereof.

62. A pharmaceutical composition comprising the siRNA molecule of claim 61 in combination with a pharmaceutically acceptable carrier.

63. The pharmaceutical composition of claim 62, further comprising a chemotherapeutic agent.

64. A kit comprising the siRNA molecule of claim 61, together with instructions for use in treating myelosuppression.

65. A method for screening for an inhibitor of a hemopoietic-restricted SH2-containing inositol-5′-phosphatase, the method comprising:
i) providing a test compound and a control compound;
ii) contacting a hemopoietic cell with the test compound or the control compound; and
iii) determining whether the test compound is capable of increasing the survival or proliferation of the hemopoietic cell compared to the control compound, wherein a test compound that increases the survival or proliferation of the hemopoietic cell compared to the control compound is an inhibitor of a SH2-containing inositol-5′-phosphatase.


SEQUENCE LISTING (428 sequences)

SEQ ID NO: 1 - DNA, 5273 bp, H. sapiens
ctagggcatggcatcccacgtgggtgtcagcacggccgcagaagaaccacttctctggcccacccatgcctgctaggccatgcttcttcagaagtggccacaactctcctgacgtctccagagccggtcattccacccagggggacttcagctgccactggacacttcaattgtacgctgcgaccagttgccaggaaggagagggctggcaagaaagccgcggcagccgtggcagggtgtatgggacggtggacggccagggcccccccctctctctctttctctctctctctcttgcttggtttctgtaatgaggaagttctccgcagctcagtttcctttccctcactgagcgcctgaaacaggaagtcagtcagttaagctggtggcagcagccgaggccaccaagaggcaacgggcggcaggttgcagtggaggggcctccgctcccctcggtggtgtgtgggtcctgggggtgcctgccggcccagccgaggaggcccacgcccaccatggtcccctgctggaaccatggcaacatcacccgctccaaggcggaggagctgctttccaggacaggcaaggacgggagcttcctcgtgcgtgccagcgagtccatctcccgggcatacgcgctctgcgtgctgtatcggaattgcgtttacacttacagaattctgcccaatgaagatgataaattcactgttcaggcatccgaaggcgtctccatgaggttcttcaccaagctggaccagctcatcgagttttacaagaaggaaaacatggggctggtgacccatctgcaataccctgtgccgctggaggaagaggacacaggcgacgaccctgaggaggacacagaaagtgtcgtgtctccacccgagctgcccccaagaaacatcccgctgactgccagctcctgtgaggccaaggaggttcctttttcaaacgagaatccccgagcgaccgagaccagccggccgagcctctccgagacattgttccagcgactgcaaagcatggacaccagtgggcttccagaagagcatcttaaggccatccaagattatttaagcactcagctcgcccaggactctgaatttgtgaagacagggtccagcagtcttcctcacctgaagaaactgaccacactgctctgcaaggagctctatggagaagtcatccggaccctcccatccctggagtctctgcagaggttatttgaccagcagctctccccgggcctccgtccacgtcctcaggttcctggtgaggccaatcccatcaacatggtgtccaagctcagccaactgacaagcctgttgtcatccattgaagacaaggtcaaggccttgctgcacgagggtcctgagtctccgcaccggccctcccttatccctccagtcacctttgaggtgaaggcagagtctctggggattcctcagaaaatgcagctcaaagtcgacgttgagtctgggaaactgatcattaagaagtccaaggatggttctgaggacaagttctacagccacaagaaaatcctgcagctcattaagtcacagaaatttctgaataagttggtgatcttggtggaaacagagaaggagaagatcctgcggaaggaatatgtttttgctgactccaaaaagagagaaggcttctgccagctcctgcagcagatgaagaacaagcactcagagcagccggagcccgacatgatcaccatcttcatcggcacctggaacatgggtaacgccccccctcccaagaagatcacgtcctggtttctctccaaggggcagggaaagacgcgggacgactctgcggactacatcccccatgacatttacgtgatcggcacccaagaggaccccctgagtgagaaggagtggctggagatcctcaaacactccctgcaagaaatcaccagtgtgacttttaaaacagtcgccatccacacgctctggaacatccgcatcgtggtgctggccaagcctgagcacgagaaccggatcagccacatctgtactgacaacgtgaagacaggcattgcaaacacactggggaacaagggagccgtgggggtgtcgttcatgttcaatggaacctccttagggttcgtcaacagccacttgacttcaggaagtgaaaagaaactcaggcgaaaccaaaactatatgaacattctccggttcctggccctgggcgacaagaagctgagtccctttaacatcactcaccgcttcacgcacctcttctggtttggggatcttaactaccgtgtggatctgcctacctgggaggcagaaaccatcatccaaaaaatcaagcagcagcagtacgcagacctcctgtcccacgaccagctgctcacagagaggagggagcagaaggtcttcctacacttcgaggaggaagaaatcacgtttgccccaacctaccgttttgagagactgactcgggacaaatacgcctacaccaagcagaaagcgacagggatgaagtacaacttgccttcctggtgtgaccgagtcctctggaagtcttatcccctggtgcacgtggtgtgtcagtcttatggcagtaccagcgacatcatgacgagtgaccacagccctgtctttgccacatttgaggcaggagtcacttcccagtttgtctccaagaacggtcccgggactgttgacagccaaggacagattgagtttctcaggtgctatgccacattgaagaccaagtcccagaccaaattctacctggagttccactcgagctgcttggagagttttgtcaagagtcaggaaggagaaaatgaagaaggaagtgagggggagctggtggtgaagtttggtgagactcttccaaagctgaagcccattatctctgaccctgagtacctgctagaccagcacatcctcatcagcatcaagtcctctgacagcgacgaatcctatggcgagggctgcattgcccttcggttagaggccacagaaacgcagctgcccatctacacgcctctcacccaccatggggagttgacaggccacttccagggggagatcaagctgcagacctctcagggcaagacgagggagaagctctatgactttgtgaagacggagcgtgatgaatccagtgggccaaagaccctgaagagcctcaccagccacgaccccatgaagcagtgggaagtcactagcagggcccctccgtgcagtggctccagcatcactgaaatcatcaaccccaactacatgggagtggggccctttgggccaccaatgcccctgcacgtgaagcagaccttgtcccctgaccagcagcccacagcctggagctacgaccagccgcccaaggactccccgctggggccctgcaggggagaaagtcctccgacacctcccggccagccgcccatatcacccaagaagtttttaccctcaacagcaaaccggggtctccctcccaggacacaggagtcaaggcccagtgacctggggaagaacgcaggggacacgctgcctcaggaggacctgccgctgacgaagcccgagatgtttgagaaccccctgtatgggtccctgagttccttccctaagcctgctcccaggaaggaccaggaatcccccaaaatgccgcggaaggaacccccgccctgcccggaacccggcatcttgtcgcccagcatcgtgctcaccaaagcccaggaggctgatcgcggcgaggggcccggcaagcaggtgcccgcgccccggctgcgctccttcacgtgctcatcctctgccgagggcagggcggccggcggggacaagagccaagggaagcccaagaccccggtcagctcccaggccccggtgccggccaagaggcccatcaagccttccagatcggaaatcaaccagcagaccccgcccaccccgacgccgcggccgccgctgccagtcaagagcccggcggtgctgcacctccagcactccaagggccgcgactaccgcgacaacaccgagctcccgcatcacggcaagcaccggccggaggaggggccaccagggcctctaggcaggactgccatgcagtgaagccctcagtgagctgccactgagtcgggagcccagaggaacggcgtgaagccactggaccctctcccgggacctcctgctggctcctcctgcccagcttcctatgcaaggctttgtgttttcaggaaagggcctagcttctgtgtggcccacagagttcactgcctgtgaggcttagcaccaagtgctgaggctggaagaaaaacgcacaccagacgggcaacaaacagtctgggtccccagctcgctcttggtacttgggaccccagtgcctcgttgagggcgccattctgaagaaaggaactgcagcgccgatttgagggtggagatatagataataataatattaataataataatggccacatggatcgaacactcatgatgtgccaagtgctgtgctaagtgctttacgaacattcgtcatatcaggatgacctcgagagctgaggctctagccacctaaaacacgtgcccaaacccaccagtttaaaacggtgtgtgttcggaggggtgaaagcattaagaagcccagtgccctcctggagtgagacaagggctcggccttaaggagctgaagagtctgggtagcttgtttagggtacaagaagcctgttctgtccagcttcagtgacacaagctgctttagctaaagtcccgcgggttccggcatggctaggctgagagcagggatctacctggcttctcagttctttggttggaaggagcaggaaatcagctcctattctccagtggagagatctggcctcagcttgggctagagatgccaaggcctgtgccaggttccctgtgccctcctcgaggtgggcagccatcaccagccacagttaagccaagccccccaacatgtattccatcgtgctggtagaagagtctttgctgttgctcccgaaagccgtgctctccagcctggctgccagggagggtgggcctcttggttccaggctcttgaaatagtgcagccttttcttcctatctctgtggctttcagctctgcttccttggttattaggagaatagatgggtgatgtctttccttatgttgctttttcaacatagcagaattaatgtagggagctaaatccagtggtgtgtgtgaatgcagaagggaatgcaccccacattcccatgatggaagtctgcgtaaccaataaattgtgcctttcttaaaaa

SEQ ID NO: 2 - protein, 1188 aa, H. sapiens
MVPCWNHGNITRSKAEELLSRTGKDGSFLVRASESISRAYALCVLYRNCVYTYRILPNEDDKFTVQASEGVSMRFFTKLDQLIEFYKKENMGLVTHLQYPVPLEEEDTGDDPEEDTESVVSPPELPPRNIPLTASSCEAKEVPFSNENPRATETSRPSLSETLFQRLQSMDTSGLPEEHLKAIQDYLSTQLAQDSEFVKTGSSSLPHLKKLTTLLCKELYGEVIRTLPSLESLQRLFDQQLSPGLRPRPQVPGEANPINMVSKLSQLTSLLSSIEDKVKALLHEGPESPHRPSLIPPVTFEVKAESLGIPQKMQLKVDVESGKLIIKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLRCYATLKTKSQTKFYLEFHSSCLESFVKSQEGENEEGSEGELVVKFGETLPKLKPIISDPEYLLDQHILISIKSSDSDESYGEGCIALRLEATETQLPIYTPLTHHGELTGHFQGEIKLQTSQGKTREKLYDFVKTERDESSGPKTLKSLTSHDPMKQWEVTSRAPPCSGSSITEIINPNYMGVGPFGPPMPLHVKQTLSPDQQPTAWSYDQPPKDSPLGPCRGESPPTPPGQPPISPKKFLPSTANRGLPPRTQESRPSDLGKNAGDTLPQEDLPLTKPEMFENPLYGSLSSFPKPAPRKDQESPKMPRKEPPPCPEPGILSPSIVLTKAQEADRGEGPGKQVPAPRLRSFTCSSSAEGRAAGGDKSQGKPKTPVSSQAPVPAKRPIKPSRSEINQQTPPTPTPRPPLPVKSPAVLHLQHSKGRDYRDNTELPHHGKHRPEEGPPGPLGRTAMQ

SEQ ID NO: 3 - DNA, 4040 bp, M. musculus
ccctggtaggagcagcagaggcaatttctgagaggcaacaggcggcaggtctcagcctagagagggccctgaactactttgctggagtgtccgtcctgggagtggctgctgacccagtccaggagacccatgcctgccatggtccctgggtggaaccatggcaacatcacccgctccaaggcagaggagctactttccagagccggcaaggacgggagcttccttgtgcgtgccagcgagtccatcccccgggcctgcgcactctgcgtgctgttccggaattgtgtttacacttacaggattctgcccaatgaggacgataaattcactgttcaggcatccgaaggtgtccccatgaggttcttcacgaagctggaccagctcatcgacttttacaagaaggaaaacatggggctggtgacccacctgcagtaccccgtgcccctggaggaggaggatgctattgatgaggctgaggaggacactgaaagtgtcatgtcaccacctgagctgcctcccagaaacattcctatgtctgccgggcccagcgaggccaaggaccttcctcttgcaacagagaacccccgagcccctgaggtcacccggctgagtctctccgagacactgtttcagcgtctacagagcatggataccagtgggcttcccgaggagcacctgaaagccatccaggattatctgagcactcagctcctcctggattccgactttttgaaaacgggctccagcaacctccctcacctgaagaagctgatgtcactgctctgcaaggagctccatggggaagtcatcaggactctgccatccctggagtctctgcagaggttgtttgaccaacagctctccccaggccttcgcccacgacctcaggtgcccggagaggccagtcccatcaccatggttgccaaactcagccaattgacaagtctgctgtcttccattgaagataaggtcaagtccttgctgcacgagggctcagaatctaccaacaggcgttcccttatccctccggtcacctttgaggtgaagtcagagtccctgggcattcctcagaaaatgcatctcaaagtggacgttgagtctgggaaactgatcgttaagaagtccaaggatggttctgaggacaagttctacagccacaaaaaaatcctgcagctcattaagtcccagaagtttctaaacaagttggtgattttggtggagacggagaaggagaaaatcctgaggaaggaatatgtttttgctgactctaagaaaagagaaggcttctgtcaactcctgcagcagatgaagaacaagcattcggagcagccagagcctgacatgatcaccatcttcattggcacttggaacatgggtaatgcaccccctcccaagaagatcacgtcctggtttctctccaaggggcagggaaagacacgggacgactctgctgactacatcccccatgacatctatgtgattggcacccaggaggatccccttggagagaaggagtggctggagctactcaggcactccctgcaagaagtcaccagcatgacatttaaaacagttgccatccacaccctctggaacattcgcatagtggtgcttgccaagccagagcatgagaatcggatcagccatatctgcactgacaacgtgaagacaggcatcgccaacaccctgggaaacaagggagcagtgggagtgtccttcatgttcaatggaacctccttggggttcgtcaacagccacttgacttctggaagtgaaaaaaagctcaggagaaatcaaaactatatgaacatcctgcggttcctggccctgggagacaagaagctaagcccatttaacatcacccaccgcttcacccacctcttctggcttggggatctcaactaccgcgtggagctgcccacttgggaggcagaggccatcatccagaagatcaagcaacagcagtattcagaccttctggcccacgaccaactgctcctggagaggaaggaccagaaggtcttcctgcactttgaggaggaagagatcaccttcgcccccacctatcgatttgaaagactgacccgggacaagtatgcatacacgaagcagaaagcaacagggatgaagtacaacttgccgtcctggtgcgaccgagtcctctggaagtcttacccgctggtgcatgtggtctgtcagtcctatggcagtaccagtgacatcatgacgagtgaccacagccctgtctttgccacgtttgaagcaggagtcacatctcaattcgtctccaagaatggtcctggcactgtagatagccaagggcagatcgagtttcttgcatgctacgccacactgaagaccaagtcccagactaagttctacttggagttccactcaagctgcttagagagttttgtcaagagtcaggaaggagagaatgaagagggaagtgaaggagagctggtggtacggtttggagagactcttcccaagctaaagcccattatctctgaccccgagtacttactggaccagcatatcctgatcagcattaaatcctctgacagtgacgagtcctatggtgaaggctgcattgcccttcgcttggagaccacagaggctcagcatcctatctacacgcctctcacccaccatggggagatgactggccacttcaggggagagattaagctgcagacctcccagggcaagatgagggagaagctctatgactttgtgaagacagagcgggatgaatccagtggaatgaaatgcttgaagaacctcaccagccatgaccctatgaggcaatgggagccttctggcagggtccctgcatgtggtgtctccagcctcaatgagatgatcaatccaaactacattggtatggggccttttggacagcccctgcatgggaaatcaaccctgtccccagatcagcaactcacagcttggagttatgaccagctacccaaagactcctccctggggcctgggaggggggagggtcctccaacccctccctcccaaccacctctgtcgccaaagaagttttcatcttccacaaccaaccgaggtccctgccccagggtgcaagaggcaagacctggggatctgggaaaggtggaagctctgctccaggaggacctgctgctgacgaagcccgagatgtttgagaacccactgtatggatccgtgagttccttccctaagctggtgcccaggaaagagcaggagtctcccaagatgctgcggaaggagcccccgccctgtccagacccaggaatctcatcacccagcatcgtgctccccaaagcccaagaggtggagagtgtcaaggggacaagcaaacaggcccctgtgcctgtccttggccccacaccccggatccgctcctttacctgttcttcttctgctgagggcagaatgaccagtggggacaagagccaagggaagcccaaggcctcagccagttcccaagccccagtgccagtcaagaggcctgtcaagccttccaggtcagaaatgagccagcagacaacacccatcccagctccacggccacccctgccagtcaagagtcctgctgtcctgcagctgcaacattccaaaggcagagactaccgtgacaacacagaactcccccaccatggcaagcaccgccaagaggaggggctgcttggcaggactgccatgcagtgagctgctggtgatcggagcctggaggaacagcacaaagcagacctgcgacctctctcaggatgcctctctcaggatgcctcttggaggacctcctgctagctcttcttgcctagcttcaagtcccaggctgtgtattttttttcaggaaacggcctcacttctctgtggtccaagaagtgtgctgctggctgccacactgtgcggcagatgctaaagctggatgacaaacgcacgccatacagacagcagacagcggcactgggtctcagaacttggattcctgggccttcttccagtcgccgttttaaagaaaggaactaacggagctgctcatccga

SEQ ID NO: 4 - protein, 1190 aa, M. Musculus
MPAMVPGWNHGNITRSKAEELLSRAGKDGSFLVRASESIPRACALCVLFRNCVYTYRILPNEDDKFTVQASEGVPMRFFTKLDQLIDFYKKENMGLVTHLQYPVPLEEEDAIDEAEEDTESVMSPPELPPRNIPMSAGPSEAKDLPLATENPRAPEVTRLSLSETLFQRLQSMDTSGLPEEHLKAIQDYLSTQLLLDSDFLKTGSSNLPHLKKLMSLLCKELHGEVIRTLPSLESLQRLFDQQLSPGLRPRPQVPGEASPITMVAKLSQLTSLLSSIEDKVKSLLHEGSESTNRRSLIPPVTFEVKSESLGIPQKMHLKVDVESGKLIVKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLGEKEWLELLRHSLQEVTSMTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWLGDLNYRVELPTWEAEAIIQKIKQQQYSDLLAHDQLLLERKDQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLACYATLKTKSQTKFYLEFHSSCLESFVKSQEGENEEGSEGELVVRFGETLPKLKPIISDPEYLLDQHILISIKSSDSDESYGEGCIALRLETTEAQHPIYTPLTHHGEMTGHFRGEIKLQTSQGKMREKLYDFVKTERDESSGMKCLKNLTSHDPMRQWEPSGRVPACGVSSLNEMINPNYIGMGPFGQPLHGKSTLSPDQQLTAWSYDQLPKDSSLGPGRGEGPPTPPSQPPLSPKKFSSSTTNRGPCPRVQEARPGDLGKVEALLQEDLLLTKPEMFENPLYGSVSSFPKLVPRKEQESPKMLRKEPPPCPDPGISSPSIVLPKAQEVESVKGTSKQAPVPVLGPTPRIRSFTCSSSAEGRMTSGDKSQGKPKASASSQAPVPVKRPVKPSRSEMSQQTTPIPAPRPPLPVKSPAVLQLQHSKGRDYRDNTELPHHGKHRQEEGLLGRTAMQ

SEQ ID NO: 5 - DNA, 21 bp, Artificial Sequence
  siRNA directed against the sequence described in GenBank Accession No. U51742
cccactagttgttgaacttta

SEQ ID NO: 6 - DNA, 21 bp, Artificial Sequence
  siRNA directed against the sequence described in GenBank Accession No. U51742
aacagggatgaagtacaactt

SEQ ID NO: 7 - DNA, 21 bp, Artificial Sequence
  siRNA directed against the sequence described in GenBank Accession No. U51742
aagtcaccagcatgacattta

SEQ ID NO: 8 - DNA, 21 bp, Artificial Sequence
  siRNA directed against the sequence described in GenBank Accession No. U51742
aaccacctctgtcgccaaaga

SEQ ID NO: 9 - DNA, 20 bp, Artificial Sequence
  siRNA directed against the sequence described in GenBank Accession No. U51742
atggactcgctggcacgcac

SEQ ID NO: 10 - DNA, 21 bp, Artificial Sequence
  siRNA directed against the sequence described in GenBank Accession No. U51742
aagagtcaggaaggagagaat

SEQ ID NO: 11 - DNA, 21 bp, Artificial Sequence
  siRNA directed against the sequence described in GenBank Accession No. NM_001017915
aagagtcaggaaggagaaaat

SEQ ID NO: 12 - DNA, 21 bp, Artificial Sequence
  siRNA directed against the sequence described in GenBank Accession No. NM_001017915
aacctccttagggttcgtcaa

SEQ ID NO: 13 - DNA, 21 bp, Artificial Sequence
  siRNA directed against the sequence described in GenBank Accession No. NM_001017915
aaggcgtctccatgaggttct

SEQ ID NO: 14 - DNA, 21 bp, Artificial Sequence
  siRNA directed against the sequence described in GenBank Accession No. NM_001017915
aagacgagggagaagctctat

SEQ ID NO: 15 - RNA, 21 nt, Artificial Sequence
  siRNA selective for SHIP1
guuuacacuuacagaauucuu

SEQ ID NO: 16 - RNA, 21 nt, Artificial Sequence
  siRNA selective for SHIP1
uucaaaugugaaugucuuaag

SEQ ID NO: 17 - RNA, 21 nt, Artificial Sequence
  siRNA selective for SHIP1
guaucggaauugcguuuacuu

SEQ ID NO: 18 - RNA, 21 nt, Artificial Sequence
  siRNA selective for SHIP1
uucauagccuuaacgcaaaug

SEQ ID NO: 19 - RNA, 21 nt, Artificial Sequence
  siRNA selective for SHIP1
ggugacccaucugcaauacuu

SEQ ID NO: 20 - RNA, 21 nt, Artificial Sequence
  siRNA selective for SHIP1
uuccacuggguagacguuaug

SEQ ID NO: 21 - RNA, 21 nt, Artificial Sequence
  siRNA selective for SHIP1
ggaagucagucaguuaagcuu

SEQ ID NO: 22 - RNA, 21 nt, Artificial Sequence
  siRNA selective for SHIP1
uuccuucagucagucaauucg

SEQ ID NO: 23 - RNA, 21 nt, Artificial Sequence
  siRNA selective for SHIP1
gaauugcguuuacacuuacuu

SEQ ID NO: 24 - RNA, 21 nt, Artificial Sequence
  siRNA selective for SHIP1
uucuuaacgcaaaugugaaug

SEQ ID NO: 25 - RNA, 21 nt, Artificial Sequence
  siRNA selective for SHIP1
gugacccaucugcaauaccuu

SEQ ID NO: 26 - RNA, 21 nt, Artificial Sequence
  siRNA selective for SHIP1
uucacuggguagacguuaugg

SEQ ID NO: 27 - RNA, 21 nt, Artificial Sequence
  siRNA selective for SHIP1
gcgcucugcgugcuguaucuu

SEQ ID NO: 28 - RNA, 21 nt, Artificial Sequence
  siRNA selective for SHIP1
uucgcgagacgcacgacauag

SEQ ID NO: 29 - RNA, 21 nt, Artificial Sequence
  siRNA selective for SHIP1
guuccagcgacugcaaagcuu

SEQ ID NO: 30 - RNA, 21 nt, Artificial Sequence
  siRNA selective for SHIP1
uucaaggucgcugacguuucg

SEQ ID NO: 31 - RNA, 21 nt, Artificial Sequence
  siRNA selective for SHIP1
gcagcucaguuuccuuuccuu

SEQ ID NO: 32 - RNA, 21 nt, Artificial Sequence
  siRNA selective for SHIP1
uucgucgagucaaaggaaagg

SEQ ID NO: 33 - RNA, 21 nt, Artificial Sequence
  siRNA selective for SHIP1
ggcggaggagcugcuuuccuu

SEQ ID NO: 34 - RNA, 21 nt, Artificial Sequence
  siRNA selective for SHIP1
uuccgccuccucgacgaaagg

SEQ ID NO: 35 - RNA, 21 nt, Artificial Sequence
  siRNA selective for SHIP1
gaugauaaauucacuguucuu

SEQ ID NO: 36 - RNA, 21 nt, Artificial Sequence
  siRNA selective for SHIP1
uucuacuauuuaagugacaag

SEQ ID NO: 37 - RNA, 21 nt, Artificial Sequence
  siRNA selective for SHIP1
gccgcagaagaaccacuucuu

SEQ ID NO: 38 - RNA, 21 nt, Artificial Sequence
  siRNA selective for SHIP1
uucggcgucuucuuggugaag

SEQ ID NO: 39 - RNA, 21 nt, Artificial Sequence
  siRNA selective for SHIP1
ggaaccauggcaacaucacuu

SEQ ID NO: 40 - RNA, 21 nt, Artificial Sequence
  siRNA selective for SHIP1
uuccuugguaccguuguagug

SEQ ID NO: 41 - RNA, 21 nt, Artificial Sequence
  siRNA selective for SHIP1
gcgccugaaacaggaagucuu

SEQ ID NO: 42 - RNA, 21 nt, Artificial Sequence
  siRNA selective for SHIP1
uucgcggacuuuguccuucag

SEQ ID NO: 43 - RNA, 21 nt, Artificial Sequence
  siRNA selective for SHIP1
gugccagcgaguccaucucuu

SEQ ID NO: 44 - RNA, 21 nt, Artificial Sequence
  siRNA selective for SHIP1
uucacggucgcucagguagag

SEQ ID NO: 45 - RNA, 21 nt, Artificial Sequence
  siRNA selective for SHIP1
guccaucucccgggcauacuu

SEQ ID NO: 46 - RNA, 21 nt, Artificial Sequence
  siRNA selective for SHIP1
uucagguagagggcccguaug

SEQ ID NO: 47 - RNA, 21 nt, Artificial Sequence
  siRNA selective for SHIP1
guacgcugcgaccaguugcuu

SEQ ID NO: 48 - RNA, 21 nt, Artificial Sequence
  siRNA selective for SHIP1
uucaugcgacgcuggucaacg

SEQ ID NO: 49 - RNA, 21 nt, Artificial Sequence
  siRNA selective for SHIP1
gcacggccgcagaagaaccuu

SEQ ID NO: 50 - RNA, 21 nt, Artificial Sequence
  siRNA selective for SHIP1
uucgugccggcgucuucuugg

SEQ ID NO: 51 - RNA, 21 nt, Artificial Sequence
  siRNA selective for SHIP1
guagucccaguugagaagcuu

SEQ ID NO: 52 - RNA, 21 nt, Artificial Sequence
  siRNA selective for SHIP1
uucaucagggucaacucuucg

SEQ ID NO: 53 - RNA, 21 nt, Artificial Sequence
  siRNA selective for SHIP1
gccugcuaggccaugcuucuu

SEQ ID NO: 54 - RNA, 21 nt, Artificial Sequence
  siRNA selective for SHIP1
uucggacgauccgguacgaag

SEQ ID NO: 55 - RNA, 21 nt, Artificial Sequence
  siRNA selective for SHIP1
ggguccagcagucuuccucuu

SEQ ID NO: 56 - RNA, 21 nt, Artificial Sequence
  siRNA selective for SHIP1
uucccaggucgucagaaggag

SEQ ID NO: 57 - RNA, 21 nt, Artificial Sequence
  siRNA selective for SHIP1
ggaggacacagaaagugucuu

SEQ ID NO: 58 - RNA, 21 nt, Artificial Sequence
  siRNA selective for SHIP1
uuccuccugugucuuucacag

SEQ ID NO: 59 - RNA, 21 nt, Artificial Sequence
  siRNA selective for SHIP1
gggcuggugacccaucugcuu

SEQ ID NO: 60 - RNA, 21 nt, Artificial Sequence
  siRNA selective for SHIP1
uucccgaccacuggguagacg

SEQ ID NO: 61 - RNA, 21 nt, Artificial Sequence
  siRNA selective for SHIP1
gaaccauggcaacaucaccuu

SEQ ID NO: 62 - RNA, 21 nt, Artificial Sequence
  siRNA selective for SHIP1
uucuugguaccguuguagugg

SEQ ID NO: 63 - RNA, 21 nt, Artificial Sequence
  siRNA selective for SHIP1
gggcuuccagaagagcaucuu

SEQ ID NO: 64 - RNA, 21 nt, Artificial Sequence
  siRNA selective for SHIP1
uucccgaaggucuucucguag

SEQ ID NO: 65 - RNA, 21 nt, Artificial Sequence
  siRNA selective for SHIP1
gcgacugcaaagcauggacuu

SEQ ID NO: 66 - RNA, 21 nt, Artificial Sequence
  siRNA selective for SHIP1
uucgcugacguuucguaccug

SEQ ID NO: 67 - RNA, 21 nt, Artificial Sequence
  siRNA selective for SHIP1
gcgugccagcgaguccaucuu

SEQ ID NO: 68 - RNA, 21 nt, Artificial Sequence
  siRNA selective for SHIP1
uucgcacggucgcucagguag

SEQ ID NO: 69 - RNA, 21 nt, Artificial Sequence
  siRNA selective for SHIP1
ggacacuucaauuguacgcuu

SEQ ID NO: 70 - RNA, 21 nt, Artificial Sequence
  siRNA selective for SHIP1
uuccugugaaguuaacaugcg

SEQ ID NO: 71 - RNA, 21 nt, Artificial Sequence
  siRNA selective for SHIP1
gaaagugucgugucuccacuu

SEQ ID NO: 72 - RNA, 21 nt, Artificial Sequence
  siRNA selective for SHIP1
uucuuucacagcacagaggug

SEQ ID NO: 73 - RNA, 21 nt, Artificial Sequence
  siRNA selective for SHIP1
ggcaaggacgggagcuuccuu

SEQ ID NO: 74 - RNA, 21 nt, Artificial Sequence
  siRNA selective for SHIP1
uuccguuccugcccucgaagg

SEQ ID NO: 75 - RNA, 21 nt, Artificial Sequence
  siRNA selective for SHIP1
gauuauuuaagcacucagcuu

SEQ ID NO: 76 - RNA, 21 nt, Artificial Sequence
  siRNA selective for SHIP1
uucuaauaaauucgugagucg

SEQ ID NO: 77 - RNA, 21 nt, Artificial Sequence
  siRNA selective for SHIP1
guccagcagucuuccucacuu

SEQ ID NO: 78 - RNA, 21 nt, Artificial Sequence
  siRNA selective for SHIP1
uucaggucgucagaaggagug

SEQ ID NO: 79 - RNA, 21 nt, Artificial Sequence
  siRNA selective for SHIP1
gaaacugaccacacugcucuu

SEQ ID NO: 80 - RNA, 21 nt, Artificial Sequence
  siRNA selective for SHIP1
uucuuugacuggugugacgag

SEQ ID NO: 81 - RNA, 21 nt, Artificial Sequence
  siRNA selective for SHIP1
gaggccaccaagaggcaacuu

SEQ ID NO: 82 - RNA, 21 nt, Artificial Sequence
  siRNA selective for SHIP1
uucuccggugguucuccguug

SEQ ID NO: 83 - RNA, 21 nt, Artificial Sequence
  siRNA selective for SHIP1
gucuagggcauggcaucccuu

SEQ ID NO: 84 - RNA, 21 nt, Artificial Sequence
  siRNA selective for SHIP1
uucagaucccguaccguaggg

SEQ ID NO: 85 - RNA, 21 nt, Artificial Sequence
  siRNA selective for SHIP1
gagcaucuuaaggccauccuu

SEQ ID NO: 86 - RNA, 21 nt, Artificial Sequence
  siRNA selective for SHIP1
uucucguagaauuccgguagg

SEQ ID NO: 87 - RNA, 21 nt, Artificial Sequence
  siRNA selective for SHIP1
ggccugucuagggcauggcuu

SEQ ID NO: 88 - RNA, 21 nt, Artificial Sequence
  siRNA selective for SHIP1
uuccggacagaucccguaccg

SEQ ID NO: 89 - RNA, 21 nt, Artificial Sequence
  siRNA selective for SHIP1
gaaguggccacaacucuccuu

SEQ ID NO: 90 - RNA, 21 nt, Artificial Sequence
  siRNA selective for SHIP1
uucuucaccgguguugagagg

SEQ ID NO: 91 - RNA, 21 nt, Artificial Sequence
  siRNA selective for SHIP1
guucuucaccaagcuggacuu

SEQ ID NO: 92 - RNA, 21 nt, Artificial Sequence
  siRNA selective for SHIP1
uucaagaagugguucgaccug

SEQ ID NO: 93 - RNA, 21 nt, Artificial Sequence
  siRNA selective for SHIP1
gugaggccaaggagguuccuu

SEQ ID NO: 94 - RNA, 21 nt, Artificial Sequence
  siRNA selective for SHIP1
uucacuccgguuccuccaagg

SEQ ID NO: 95 - RNA, 21 nt, Artificial Sequence
  siRNA selective for SHIP1
gaaacaucccgcugacugcuu

SEQ ID NO: 96 - RNA, 21 nt, Artificial Sequence
  siRNA selective for SHIP1
uucuuuguagggcgacugacg

SEQ ID NO: 97 - RNA, 21 nt, Artificial Sequence
  siRNA selective for SHIP1
ggcauccgaaggcgucuccuu

SEQ ID NO: 98 - RNA, 21 nt, Artificial Sequence
  siRNA selective for SHIP1
uuccguaggcuuccgcagagg

SEQ ID NO: 99 - RNA, 21 nt, Artificial Sequence
  siRNA selective for SHIP1
ggcaucccacgugggugucuu

SEQ ID NO: 100 - RNA, 21 nt, Artificial Sequence
  siRNA selective for SHIP1
uuccguagggugcacccacag

SEQ ID NO: 101 - RNA, 21 nt, Artificial Sequence
  siRNA selective for SHIP1
gacugcaaagcauggacacuu

SEQ ID NO: 102 - RNA, 21 nt, Artificial Sequence
  siRNA selective for SHIP1
uucugacguuucguaccugug

SEQ ID NO: 103 - RNA, 21 nt, Artificial Sequence
  siRNA selective for SHIP1
gccgcuggaggaagaggacuu

SEQ ID NO: 104 - RNA, 21 nt, Artificial Sequence
  siRNA selective for SHIP1
uucggcgaccuccuucuccug

SEQ ID NO: 105 - RNA, 21 nt, Artificial Sequence
  siRNA selective for SHIP1
gaggacacaggcgacgaccuu

SEQ ID NO: 106 - RNA, 21 nt, Artificial Sequence
  siRNA selective for SHIP1
uucuccuguguccgcugcugg

SEQ ID NO: 107 - RNA, 21 nt, Artificial Sequence
  siRNA selective for SHIP1
gagacauuguuccagcgacuu

SEQ ID NO: 108 - RNA, 21 nt, Artificial Sequence
  siRNA selective for SHIP1
uucucuguaacaaggucgcug

SEQ ID NO: 109 - RNA, 21 nt, Artificial Sequence
  siRNA selective for SHIP1
gacgggagcuuccucgugcuu

SEQ ID NO: 110 - RNA, 21 nt, Artificial Sequence
  siRNA selective for SHIP1
uucugcccucgaaggagcacg

SEQ ID NO: 111 - RNA, 21 nt, Artificial Sequence
  siRNA selective for SHIP1
gugucuccacccgagcugcuu

SEQ ID NO: 112 - RNA, 21 nt, Artificial Sequence
  siRNA selective for SHIP1
uucacagaggugggcucgacg

SEQ ID NO: 113 - RNA, 21 nt, Artificial Sequence
  siRNA selective for SHIP1
gagggcuggcaagagagccuu

SEQ ID NO: 114 - RNA, 21 nt, Artificial Sequence
  siRNA selective for SHIP1
uucucccgaccguucucucgg

SEQ ID NO: 115 - RNA, 21 nt, Artificial Sequence
  siRNA selective for SHIP1
gcugcuuuccaggacaggcuu

SEQ ID NO: 116 - RNA, 21 nt, Artificial Sequence
  siRNA selective for SHIP1
uucgacgaaagguccuguccg

SEQ ID NO: 117 - RNA, 21 nt, Artificial Sequence
  siRNA selective for SHIP1
ggaggaagaggacacaggcuu

SEQ ID NO: 118 - RNA, 21 nt, Artificial Sequence
  siRNA selective for SHIP1
uuccuccuucuccuguguccg

SEQ ID NO: 119 - RNA, 21 nt, Artificial Sequence
  siRNA selective for SHIP1
gagggagagcagaaggcucuu

SEQ ID NO: 120 - RNA, 21 nt, Artificial Sequence
  siRNA selective for SHIP1
uucucccucucgucuuccgag

SEQ ID NO: 121 - RNA, 21 nt, Artificial Sequence
  siRNA selective for SHIP1
gcucaguuuccuuucccucuu

SEQ ID NO: 122 - RNA, 21 nt, Artificial Sequence
  siRNA selective for SHIP1
uucgagucaaaggaaagggag

SEQ ID NO: 123 - RNA, 21 nt, Artificial Sequence
  siRNA selective for SHIP1
gccagcgaguccaucucccuu

SEQ ID NO: 124 - RNA, 21 nt, Artificial Sequence
  siRNA selective for SHIP1
uucggucgcucagguagaggg

SEQ ID NO: 125 - RNA, 21 nt, Artificial Sequence
  siRNA selective for SHIP1
gaagaaccacuucucuggcuu

SEQ ID NO: 126 - RNA, 21 nt, Artificial Sequence
  siRNA selective for SHIP1
uucuucuuggugaagagaccg

SEQ ID NO: 127 - RNA, 21 nt, Artificial Sequence
  siRNA selective for SHIP1
gagcuuccucgugcgugccuu

SEQ ID NO: 128 - RNA, 21 nt, Artificial Sequence
  siRNA selective for SHIP1
uucucgaaggagcacgcacgg

SEQ ID NO: 129 - RNA, 21 nt, Artificial Sequence
  siRNA selective for SHIP1
gucgugucuccacccgagcuu

SEQ ID NO: 130 - RNA, 21 nt, Artificial Sequence
  siRNA selective for SHIP1
uucagcacagaggugggcucg

SEQ ID NO: 131 - RNA, 21 nt, Artificial Sequence
  siRNA selective for SHIP1
gcauacgcgcucugcgugcuu

SEQ ID NO: 132 - RNA, 21 nt, Artificial Sequence
  siRNA selective for SHIP1
uucguaugcgcgagacgcacg

SEQ ID NO: 133 - RNA, 21 nt, Artificial Sequence
  siRNA selective for SHIP1
ggagcuuccucgugcgugcuu

SEQ ID NO: 134 - RNA, 21 nt, Artificial Sequence
  siRNA selective for SHIP1
uuccucgaaggagcacgcacg

SEQ ID NO: 135 - RNA, 21 nt, Artificial Sequence
  siRNA selective for SHIP1
gacguccucagagccggucuu

SEQ ID NO: 136 - RNA, 21 nt, Artificial Sequence
  siRNA selective for SHIP1
uucugcagaggucucggccag

SEQ ID NO: 137 - RNA, 21 nt, Artificial Sequence
  siRNA selective for SHIP1
ggaagaggacacaggcgacuu

SEQ ID NO: 138 - RNA, 21 nt, Artificial Sequence
  siRNA selective for SHIP1
uuccuucuccuguguccgcug

SEQ ID NO: 139 - RNA, 21 nt, Artificial Sequence
  siRNA selective for SHIP1
ggagguucagggugggugcuu

SEQ ID NO: 140 - RNA, 21 nt, Artificial Sequence
  siRNA selective for SHIP1
uuccuccaagucccacccacg

SEQ ID NO: 141 - RNA, 21 nt, Artificial Sequence
  siRNA selective for SHIP1
gacaggcaaggacgggagcuu

SEQ ID NO: 142 - RNA, 21 nt, Artificial Sequence
  siRNA selective for SHIP1
uucuguccguuccugcccucg

SEQ ID NO: 143 - DNA, 21 bp, Artificial Sequence
  siRNA selective for SHIP1
gcuggaccagcucaucgagtt

SEQ ID NO: 144 - DNA, 25 bp, Artificial Sequence
  siRNA selective for SHIP1
aagctggaccagctcatcgagttas

SEQ ID NO: 145 - DNA, 21 bp, Artificial Sequence
  siRNA selective for SHIP1
ttcgaccuggucgaguagcuc

SEQ ID NO: 146 - DNA, 21 bp, Artificial Sequence
  siRNA selective for SHIP1
agcauggacaccagugggctt

SEQ ID NO: 147 - DNA, 25 bp, Artificial Sequence
  siRNA selective for SHIP1
aaagcatggacaccagtgggcttas

SEQ ID NO: 148 - DNA, 21 bp, Artificial Sequence
  siRNA selective for SHIP1
ttucguaccuguggucacccg

SEQ ID NO: 149 - DNA, 19 bp, Artificial Sequence
  siRNA selective for SHIP1
gcaaggagctctatgggta

SEQ ID NO: 150 - DNA, 19 bp, Artificial Sequence
  siRNA selective for SHIP1
ggaattgcgtttacactta

SEQ ID NO: 151 - DNA, 19 bp, Artificial Sequence
  siRNA selective for SHIP1
ggagagggctggcaagaga

SEQ ID NO: 152 - DNA, 19 bp, Artificial Sequence
  siRNA selective for SHIP1
gcccaatgaagatgataaa

SEQ ID NO: 153 - DNA, 19 bp, Artificial Sequence
  siRNA selective for SHIP1
acaggaagtcagtcagtta

SEQ ID NO: 154 - DNA, 19 bp, Artificial Sequence
  siRNA selective for SHIP1
gcgtttacacttacagaat

SEQ ID NO: 155 - DNA, 19 bp, Artificial Sequence
  siRNA selective for SHIP1
agacattgttccagcgact

SEQ ID NO: 156 - DNA, 19 bp, Artificial Sequence
  siRNA selective for SHIP1
caaggagctctatgggtaa

SEQ ID NO: 157 - DNA, 19 bp, Artificial Sequence
  siRNA selective for SHIP1
ggaaggagagggctggcaa

SEQ ID NO: 158 - DNA, 19 bp, Artificial Sequence
  siRNA selective for SHIP1
cctgaggaggacacagaaa

SEQ ID NO: 159 - DNA, 19 bp, Artificial Sequence
  siRNA selective for SHIP1
tgaaacaggaagtcagtca

SEQ ID NO: 160 - DNA, 19 bp, Artificial Sequence
  siRNA selective for SHIP1
ccatgaggttcttcaccaa

SEQ ID NO: 161 - DNA, 19 bp, Artificial Sequence
  siRNA selective for SHIP1
ggaccagctcatcgagttt

SEQ ID NO: 162 - DNA, 19 bp, Artificial Sequence
  siRNA selective for SHIP1
tcactgagcgcctgaaaca

SEQ ID NO: 163 - DNA, 19 bp, Artificial Sequence
  siRNA selective for SHIP1
ccgtagtcccagttgagaa

SEQ ID NO: 164 - DNA, 19 bp, Artificial Sequence
  siRNA selective for SHIP1
ctgtatcggaattgcgttt

SEQ ID NO: 165 - DNA, 19 bp, Artificial Sequence
  siRNA selective for SHIP1
cggaattgcgtttacactt

SEQ ID NO: 166 - DNA, 19 bp, Artificial Sequence
  siRNA selective for SHIP1
aggaagaggacacaggcga

SEQ ID NO: 167 - DNA, 19 bp, Artificial Sequence
  siRNA selective for SHIP1
ccagttgccaggaaggaga

SEQ ID NO: 168 - DNA, 19 bp, Artificial Sequence
  siRNA selective for SHIP1
caggaagtcagtcagttaa

SEQ ID NO: 169 - DNA, 19 bp, Artificial Sequence
  siRNA selective for SHIP1
tgcccaatgaagatgataa

SEQ ID NO: 170 - DNA, 19 bp, Artificial Sequence
  siRNA selective for SHIP1
tggtttctgtaatgaggaa

SEQ ID NO: 171 - DNA, 19 bp, Artificial Sequence
  siRNA selective for SHIP1
cgtttacacttacagaatt

SEQ ID NO: 172 - DNA, 19 bp, Artificial Sequence
  siRNA selective for SHIP1
gtttacacttacagaattc

SEQ ID NO: 173 - DNA, 19 bp, Artificial Sequence
  siRNA selective for SHIP1
tcatcgagttttacaagaa

SEQ ID NO: 174 - DNA, 19 bp, Artificial Sequence
  siRNA selective for SHIP1
ctgtgccgctggaggaaga

SEQ ID NO: 175 - DNA, 19 bp, Artificial Sequence
  siRNA selective for SHIP1
ccaagaaacatcccgctga

SEQ ID NO: 176 - DNA, 19 bp, Artificial Sequence
  siRNA selective for SHIP1
cgcccaggactctgaattt

SEQ ID NO: 177 - DNA, 19 bp, Artificial Sequence
  siRNA selective for SHIP1
actctgaatttgtgaagac

SEQ ID NO: 178 - DNA, 19 bp, Artificial Sequence
  siRNA selective for SHIP1
caggcaaggacgggagctt

SEQ ID NO: 179 - DNA, 19 bp, Artificial Sequence
  siRNA selective for SHIP1
catggacaccagtgggctt

SEQ ID NO: 180 - DNA, 19 bp, Artificial Sequence
  siRNA selective for SHIP1
cttaaggccatccaagatt

SEQ ID NO: 181 - DNA, 19 bp, Artificial Sequence
  siRNA selective for SHIP1
ccaagattatttaagcact

SEQ ID NO: 182 - DNA, 19 bp, Artificial Sequence
  siRNA selective for SHIP1
caagattatttaagcactc

SEQ ID NO: 183 - DNA, 19 bp, Artificial Sequence
  siRNA selective for SHIP1
gaattctgcccaatgaaga

SEQ ID NO: 184 - DNA, 19 bp, Artificial Sequence
  siRNA selective for SHIP1
tgaggaggacacagaaagt

SEQ ID NO: 185 - DNA, 19 bp, Artificial Sequence
  siRNA selective for SHIP1
cacagaaagtgtcgtgtct

SEQ ID NO: 186 - DNA, 19 bp, Artificial Sequence
  siRNA selective for SHIP1
ttaaggccatccaagatta

SEQ ID NO: 187 - DNA, 19 bp, Artificial Sequence
  siRNA selective for SHIP1
tgaagaaactgaccacact

SEQ ID NO: 188 - DNA, 19 bp, Artificial Sequence
  siRNA selective for SHIP1
gctggtgacccatctgcaa

SEQ ID NO: 189 - DNA, 19 bp, Artificial Sequence
  siRNA selective for SHIP1
ggaagaggacacaggcgac

SEQ ID NO: 190 - DNA, 19 bp, Artificial Sequence
  siRNA selective for SHIP1
cctgtgaggccaaggaggt

SEQ ID NO: 191 - DNA, 19 bp, Artificial Sequence
  siRNA selective for SHIP1
ctgaagaaactgaccacac

SEQ ID NO: 192 - DNA, 19 bp, Artificial Sequence
  siRNA selective for SHIP1
tgaccacactgctctgcaa

SEQ ID NO: 193 - DNA, 19 bp, Artificial Sequence
  siRNA selective for SHIP1
gtttctgtaatgaggaagt

SEQ ID NO: 194 - DNA, 19 bp, Artificial Sequence
  siRNA selective for SHIP1
cctgctggaaccatggcaa

SEQ ID NO: 195 - DNA, 19 bp, Artificial Sequence
  siRNA selective for SHIP1
ggagctgctttccaggaca

SEQ ID NO: 196 - DNA, 19 bp, Artificial Sequence
  siRNA selective for SHIP1
cgactgcaaagcatggaca

SEQ ID NO: 197 - DNA, 19 bp, Artificial Sequence
  siRNA selective for SHIP1
ctgcaaagcatggacacca

SEQ ID NO: 198 - DNA, 23 bp, Artificial Sequence
  siRNA selective for SHIP1
aaagcatggacaccagtgggctt

SEQ ID NO: 199 - DNA, 23 bp, Artificial Sequence
  siRNA selective for SHIP1
aagctggaccagctcatcgagtt

SEQ ID NO: 200 - DNA, 23 bp, Artificial Sequence
  siRNA selective for SHIP1
aagctgtgcccccttgggtgttt

SEQ ID NO: 201 - DNA, 23 bp, Artificial Sequence
  siRNA selective for SHIP1
aagaaactgaccacactgctctg

SEQ ID NO: 202 - DNA, 23 bp, Artificial Sequence
  siRNA selective for SHIP1
aaggcgtctccatgaggttcttc

SEQ ID NO: 203 - DNA, 23 bp, Artificial Sequence
  siRNA selective for SHIP1
aatgaggaagttctccgcagctc

SEQ ID NO: 204 - DNA, 23 bp, Artificial Sequence
  siRNA selective for SHIP1
aagcatggacaccagtgggcttc

SEQ ID NO: 205 - DNA, 23 bp, Artificial Sequence
  siRNA selective for SHIP1
aaacatggggctggtgacccatc

SEQ ID NO: 206 - DNA, 23 bp, Artificial Sequence
  siRNA selective for SHIP1
aaccatggcaacatcacccgctc

SEQ ID NO: 207 - DNA, 23 bp, Artificial Sequence
  siRNA selective for SHIP1
aacatcccgctgactgccagctc

SEQ ID NO: 208 - DNA, 23 bp, Artificial Sequence
  siRNA selective for SHIP1
aagaggcaacgggcggcaggttg

SEQ ID NO: 209 - DNA, 23 bp, Artificial Sequence
  siRNA selective for SHIP1
aagattatttaagcactcagctc

SEQ ID NO: 210 - DNA, 23 bp, Artificial Sequence
  siRNA selective for SHIP1
aattctgcccaatgaagatgata

SEQ ID NO: 211 - DNA, 23 bp, Artificial Sequence
  siRNA selective for SHIP1
gaaggcgtctccatgaggttctt

SEQ ID NO: 212 - DNA, 23 bp, Artificial Sequence
  siRNA selective for SHIP1
cagctcgcccaggactctgaatt

SEQ ID NO: 213 - DNA, 23 bp, Artificial Sequence
  siRNA selective for SHIP1
cagttgagaagctgtgccccctt

SEQ ID NO: 214 - DNA, 23 bp, Artificial Sequence
  siRNA selective for SHIP1
gaagctgtgcccccttgggtgtt

SEQ ID NO: 215 - DNA, 23 bp, Artificial Sequence
  siRNA selective for SHIP1
caagaggcaacgggcggcaggtt

SEQ ID NO: 216 - DNA, 23 bp, Artificial Sequence
  siRNA selective for SHIP1
cagggcccccccctctctctctt

SEQ ID NO: 217 - DNA, 23 bp, Artificial Sequence
  siRNA selective for SHIP1
caggaagtcagtcagttaagctg

SEQ ID NO: 218 - DNA, 23 bp, Artificial Sequence
  siRNA selective for SHIP1
caattgtacgctgcgaccagttg

SEQ ID NO: 219 - DNA, 23 bp, Artificial Sequence
  siRNA selective for SHIP1
cagaagagcatcttaaggccatc

SEQ ID NO: 220 - DNA, 23 bp, Artificial Sequence
  siRNA selective for SHIP1
gaagtcagtcagttaagctggtg

SEQ ID NO: 221 - DNA, 23 bp, Artificial Sequence
  siRNA selective for SHIP1
gaggccaaggaggttcctttttc

SEQ ID NO: 222 - DNA, 23 bp, Artificial Sequence
  siRNA selective for SHIP1
gaggacacagaaagtgtcgtgtc

SEQ ID NO: 223 - DNA, 23 bp, Artificial Sequence
  siRNA selective for SHIP1
gaagttctccgcagctcagtttc

SEQ ID NO: 224 - DNA, 23 bp, Artificial Sequence
  siRNA selective for SHIP1
taggccatgcttcttcagaagtg

SEQ ID NO: 225 - DNA, 23 bp, Artificial Sequence
  siRNA selective for SHIP1
cagaattctgcccaatgaagatg

SEQ ID NO: 226 - DNA, 23 bp, Artificial Sequence
  siRNA selective for SHIP1
cacttacagaattctgcccaatg

SEQ ID NO: 227 - DNA, 23 bp, Artificial Sequence
  siRNA selective for SHIP1
cagaagtggccacaactctcctg

SEQ ID NO: 228 - DNA, 23 bp, Artificial Sequence
  siRNA selective for SHIP1
cagtgggcttccagaagagcatc

SEQ ID NO: 229 - DNA, 23 bp, Artificial Sequence
  siRNA selective for SHIP1
gaagacagggtccagcagtcttc

SEQ ID NO: 230 - DNA, 23 bp, Artificial Sequence
  siRNA selective for SHIP1
catggtcccctgctggaaccatg

SEQ ID NO: 231 - DNA, 23 bp, Artificial Sequence
  siRNA selective for SHIP1
cacggccgcagaagaaccacttc

SEQ ID NO: 232 - DNA, 23 bp, Artificial Sequence
  siRNA selective for SHIP1
catcttaaggccatccaagatta

SEQ ID NO: 233 - DNA, 23 bp, Artificial Sequence
  siRNA selective for SHIP1
gacagggtccagcagtcttcctc

SEQ ID NO: 234 - DNA, 23 bp, Artificial Sequence
  siRNA selective for SHIP1
gataaattcactgttcaggcatc

SEQ ID NO: 235 - DNA, 23 bp, Artificial Sequence
  siRNA selective for SHIP1
gacgtctccagagccggtcattc

SEQ ID NO: 236 - DNA, 23 bp, Artificial Sequence
  siRNA selective for SHIP1
cactcagctcgcccaggactctg

SEQ ID NO: 237 - DNA, 23 bp, Artificial Sequence
  siRNA selective for SHIP1
cagggggacttcagctgccactg

SEQ ID NO: 238 - DNA, 23 bp, Artificial Sequence
  siRNA selective for SHIP1
catccaagattatttaagcactc

SEQ ID NO: 239 - DNA, 23 bp, Artificial Sequence
  siRNA selective for SHIP1
caggcaaggacgggagcttcctc

SEQ ID NO: 240 - DNA, 23 bp, Artificial Sequence
  siRNA selective for SHIP1
cagaaggctcgggggcctgtcta

SEQ ID NO: 241 - DNA, 23 bp, Artificial Sequence
  siRNA selective for SHIP1
gagaagctgtgcccccttgggtg

SEQ ID NO: 242 - DNA, 23 bp, Artificial Sequence
  siRNA selective for SHIP1
tatcggaattgcgtttacactta

SEQ ID NO: 243 - DNA, 23 bp, Artificial Sequence
  siRNA selective for SHIP1
caatgaagatgataaattcactg

SEQ ID NO: 244 - DNA, 23 bp, Artificial Sequence
  siRNA selective for SHIP1
gagaccagccggccgagcctctc

SEQ ID NO: 245 - DNA, 23 bp, Artificial Sequence
  siRNA selective for SHIP1
gacggccagggcccccccctctc

SEQ ID NO: 246 - DNA, 21 bp, Artificial Sequence
  siRNA selective for SHIP1
aagctgtgcccccttgggtgt

SEQ ID NO: 247 - DNA, 21 bp, Artificial Sequence
  siRNA selective for SHIP1
aagccctgagggagagcagaa

SEQ ID NO: 248 - DNA, 21 bp, Artificial Sequence
  siRNA selective for SHIP1
aaggctcgggggcctgtctag

SEQ ID NO: 249 - DNA, 21 bp, Artificial Sequence
  siRNA selective for SHIP1
aagaaccacttctctggccca

SEQ ID NO: 250 - DNA, 21 bp, Artificial Sequence
  siRNA selective for SHIP1
aaccacttctctggcccaccc

SEQ ID NO: 251 - DNA, 21 bp, Artificial Sequence
  siRNA selective for SHIP1
aagtggccacaactctcctga

SEQ ID NO: 252 - DNA, 21 bp, Artificial Sequence
  siRNA selective for SHIP1
aactctcctgacgtctccaga

SEQ ID NO: 253 - DNA, 21 bp, Artificial Sequence
  siRNA selective for SHIP1
aattgtacgctgcgaccagtt

SEQ ID NO: 254 - DNA, 21 bp, Artificial Sequence
  siRNA selective for SHIP1
aaggagagggctggcaagaga

SEQ ID NO: 255 - DNA, 21 bp, Artificial Sequence
  siRNA selective for SHIP1
aagagagccgcggcagccgtg

SEQ ID NO: 256 - DNA, 21 bp, Artificial Sequence
  siRNA selective for SHIP1
aatgaggaagttctccgcagc

SEQ ID NO: 257 - DNA, 21 bp, Artificial Sequence
  siRNA selective for SHIP1
aagttctccgcagctcagttt

SEQ ID NO: 258 - DNA, 21 bp, Artificial Sequence
  siRNA selective for SHIP1
aaacaggaagtcagtcagtta

SEQ ID NO: 259 - DNA, 21 bp, Artificial Sequence
  siRNA selective for SHIP1
aagtcagtcagttaagctggt

SEQ ID NO: 260 - DNA, 21 bp, Artificial Sequence
  siRNA selective for SHIP1
aagctggtggcagcagccgag

SEQ ID NO: 261 - DNA, 21 bp, Artificial Sequence
  siRNA selective for SHIP1
aagaggcaacgggcggcaggt

SEQ ID NO: 262 - DNA, 21 bp, Artificial Sequence
  siRNA selective for SHIP1
aacgggcggcaggttgcagtg

SEQ ID NO: 263 - DNA, 21 bp, Artificial Sequence
  siRNA selective for SHIP1
aaccatggcaacatcacccgc

SEQ ID NO: 264 - DNA, 21 bp, Artificial Sequence
  siRNA selective for SHIP1
aacatcacccgctccaaggcg

SEQ ID NO: 265 - DNA, 21 bp, Artificial Sequence
  siRNA selective for SHIP1
aaggcggaggagctgctttcc

SEQ ID NO: 266 - DNA, 21 bp, Artificial Sequence
  siRNA selective for SHIP1
aaggacgggagcttcctcgtg

SEQ ID NO: 267 - DNA, 21 bp, Artificial Sequence
  siRNA selective for SHIP1
aattgcgtttacacttacaga

SEQ ID NO: 268 - DNA, 21 bp, Artificial Sequence
  siRNA selective for SHIP1
aattctgcccaatgaagatga

SEQ ID NO: 269 - DNA, 21 bp, Artificial Sequence
  siRNA selective for SHIP1
aatgaagatgataaattcact

SEQ ID NO: 270 - DNA, 21 bp, Artificial Sequence
  siRNA selective for SHIP1
aagatgataaattcactgttc

SEQ ID NO: 271 - DNA, 21 bp, Artificial Sequence
  siRNA selective for SHIP1
aaattcactgttcaggcatcc

SEQ ID NO: 272 - DNA, 21 bp, Artificial Sequence
  siRNA selective for SHIP1
aaggcgtctccatgaggttct

SEQ ID NO: 273 - DNA, 20 bp, Artificial Sequence
  siRNA selective for SHIP1
agctggaccagctcatcgag

SEQ ID NO: 274 - DNA, 21 bp, Artificial Sequence
  siRNA selective for SHIP1
aacatggggctggtgacccat

SEQ ID NO: 275 - DNA, 21 bp, Artificial Sequence
  siRNA selective for SHIP1
aataccctgtgccgctggagg

SEQ ID NO: 276 - DNA, 21 bp, Artificial Sequence
  siRNA selective for SHIP1
aagaggacacaggcgacgacc

SEQ ID NO: 277 - DNA, 21 bp, Artificial Sequence
  siRNA selective for SHIP1
aaagtgtcgtgtctccacccg

SEQ ID NO: 278 - DNA, 21 bp, Artificial Sequence
  siRNA selective for SHIP1
aagaaacatcccgctgactgc

SEQ ID NO: 279 - DNA, 21 bp, Artificial Sequence
  siRNA selective for SHIP1
aaacatcccgctgactgccag

SEQ ID NO: 280 - DNA, 21 bp, Artificial Sequence
  siRNA selective for SHIP1
aaacgagaatccccgagcgac

SEQ ID NO: 281 - DNA, 21 bp, Artificial Sequence
  siRNA selective for SHIP1
aatccccgagcgaccgagacc

SEQ ID NO: 282 - DNA, 21 bp, Artificial Sequence
  siRNA selective for SHIP1
aaagcatggacaccagtgggc

SEQ ID NO: 283 - DNA, 21 bp, Artificial Sequence
  siRNA selective for SHIP1
aagagcatcttaaggccatcc

SEQ ID NO: 284 - DNA, 21 bp, Artificial Sequence
  siRNA selective for SHIP1
aaggccatccaagattattta

SEQ ID NO: 285 - DNA, 21 bp, Artificial Sequence
  siRNA selective for SHIP1
aagattatttaagcactcagc

SEQ ID NO: 286 - DNA, 21 bp, Artificial Sequence
  siRNA selective for SHIP1
aagcactcagctcgcccagga

SEQ ID NO: 287 - DNA, 21 bp, Artificial Sequence
  siRNA selective for SHIP1
aatttgtgaagacagggtcca

SEQ ID NO: 288 - DNA, 21 bp, Artificial Sequence
  siRNA selective for SHIP1
aagacagggtccagcagtctt

SEQ ID NO: 289 - DNA, 21 bp, Artificial Sequence
  siRNA selective for SHIP1
aagaaactgaccacactgctc

SEQ ID NO: 290 - DNA, 21 bp, Artificial Sequence
  siRNA selective for SHIP1
aaactgaccacactgctctgc

SEQ ID NO: 291 - DNA, 21 bp, Artificial Sequence
  siRNA selective for SHIP1
gtcggtttctatctacttaaa

SEQ ID NO: 292 - DNA, 21 bp, Artificial Sequence
  siRNA selective for SHIP1
tacgaaaggacaagagaatta

SEQ ID NO: 293 - DNA, 21 bp, Artificial Sequence
  siRNA selective for SHIP1
gctaagacttcaccagtcaaa

SEQ ID NO: 294 - DNA, 21 bp, Artificial Sequence
  siRNA selective for SHIP1
ataacttgaccaacggaacaa

SEQ ID NO: 295 - DNA, 21 bp, Artificial Sequence
  siRNA selective for SHIP1
atcctattctagagtccatat

SEQ ID NO: 296 - DNA, 21 bp, Artificial Sequence
  siRNA selective for SHIP1
ccaatggtgcagccgctatta

SEQ ID NO: 297 - DNA, 21 bp, Artificial Sequence
  siRNA selective for SHIP1
tctgagttcagaccggagtaa

SEQ ID NO: 298 - DNA, 21 bp, Artificial Sequence
  siRNA selective for SHIP1
cagtcaaagcgaactactata

SEQ ID NO: 299 - DNA, 21 bp, Artificial Sequence
  siRNA selective for SHIP1
cgctattaaaggttcgtttgt

SEQ ID NO: 300 - DNA, 21 bp, Artificial Sequence
  siRNA selective for SHIP1
gagtaatccaggtcggtttct

SEQ ID NO: 301 - DNA, 21 bp, Artificial Sequence
  siRNA selective for SHIP1
gactctgaatttgtgaagaca

SEQ ID NO: 302 - DNA, 21 bp, Artificial Sequence
  siRNA selective for SHIP1
tcctgagacttaaacacttct

SEQ ID NO: 303 - DNA, 21 bp, Artificial Sequence
  siRNA selective for SHIP1
ctttctctctctctctcttgc

SEQ ID NO: 304 - DNA, 21 bp, Artificial Sequence
  siRNA selective for SHIP1
gagaaagagagagagagagaa

SEQ ID NO: 305 - DNA, 21 bp, Artificial Sequence
  siRNA selective for SHIP1
cttaaggccatccaagattat

SEQ ID NO: 306 - DNA, 21 bp, Artificial Sequence
  siRNA selective for SHIP1
tagaattccggtaggttctaa

SEQ ID NO: 307 - DNA, 21 bp, Artificial Sequence
  siRNA selective for SHIP1
cacctgaagaaactgaccaca

SEQ ID NO: 308 - DNA, 21 bp, Artificial Sequence
  siRNA selective for SHIP1
gagtggacttctttgactggt

SEQ ID NO: 309 - DNA, 21 bp, Artificial Sequence
  siRNA selective for SHIP1
ctctctctcttgcttggtttc

SEQ ID NO: 310 - DNA, 21 bp, Artificial Sequence
  siRNA selective for SHIP1
gagagagagagaacgaaccaa

SEQ ID NO: 311 - DNA, 21 bp, Artificial Sequence
  siRNA selective for SHIP1
gcgtttacacttacagaattc

SEQ ID NO: 312 - DNA, 21 bp, Artificial Sequence
  siRNA selective for SHIP1
aacgcaaatgtgaatgtctta

SEQ ID NO: 313 - DNA, 21 bp, Artificial Sequence
  siRNA selective for SHIP1
cgtttacacttacagaattct

SEQ ID NO: 314 - DNA, 21 bp, Artificial Sequence
  siRNA selective for SHIP1
acgcaaatgtgaatgtcttaa

SEQ ID NO: 315 - DNA, 21 bp, Artificial Sequence
  siRNA selective for SHIP1
gccatccaagattatttaagc

SEQ ID NO: 316 - DNA, 21 bp, Artificial Sequence
  siRNA selective for SHIP1
tccggtaggttctaataaatt

SEQ ID NO: 317 - DNA, 21 bp, Artificial Sequence
  siRNA selective for SHIP1
cctgaagaaactgaccacact

SEQ ID NO: 318 - DNA, 21 bp, Artificial Sequence
  siRNA selective for SHIP1
gtggacttctttgactggtgt

SEQ ID NO: 319 - DNA, 21 bp, Artificial Sequence
  siRNA selective for SHIP1
caggactctgaatttgtgaag

SEQ ID NO: 320 - DNA, 21 bp, Artificial Sequence
  siRNA selective for SHIP1
gggtcctgagacttaaacact

SEQ ID NO: 321 - DNA, 21 bp, Artificial Sequence
  siRNA selective for SHIP1
tctctctctctctcttgcttg

SEQ ID NO: 322 - DNA, 21 bp, Artificial Sequence
  siRNA selective for SHIP1
aaagagagagagagagaacga

SEQ ID NO: 323 - DNA, 21 bp, Artificial Sequence
  siRNA selective for SHIP1
tgcccaatgaagatgataaat

SEQ ID NO: 324 - DNA, 21 bp, Artificial Sequence
  siRNA selective for SHIP1
agacgggttacttctactatt

SEQ ID NO: 325 - DNA, 21 bp, Artificial Sequence
  siRNA selective for SHIP1
ggaggagctgctttccaggac

SEQ ID NO: 326 - DNA, 21 bp, Artificial Sequence
  siRNA selective for SHIP1
cgcctcctcgacgaaaggtcc

SEQ ID NO: 327 - DNA, 21 bp, Artificial Sequence
  siRNA selective for SHIP1
cccccctctctctctttctct

SEQ ID NO: 328 - DNA, 21 bp, Artificial Sequence
  siRNA selective for SHIP1
ggggggggagagagagaaaga

SEQ ID NO: 329 - DNA, 21 bp, Artificial Sequence
  siRNA selective for SHIP1
agtttcctttccctcactgag

SEQ ID NO: 330 - DNA, 21 bp, Artificial Sequence
  siRNA selective for SHIP1
agtcaaaggaaagggagtgac

SEQ ID NO: 331 - DNA, 21 bp, Artificial Sequence
  siRNA selective for SHIP1
ccccctctctctctttctctc

SEQ ID NO: 332 - DNA, 21 bp, Artificial Sequence
  siRNA selective for SHIP1
gggggggagagagagaaagag

SEQ ID NO: 333 - DNA, 21 bp, Artificial Sequence
  siRNA selective for SHIP1
ggtggtgtgtgggtcctgggg

SEQ ID NO: 334 - DNA, 21 bp, Artificial Sequence
  siRNA selective for SHIP1
agccaccacacacccaggacc

SEQ ID NO: 335 - DNA, 21 bp, Artificial Sequence
  siRNA selective for SHIP1
ccgaggaggcccacgcccacc

SEQ ID NO: 336 - DNA, 21 bp, Artificial Sequence
  siRNA selective for SHIP1
ccggctcctccgggtgcgggt

SEQ ID NO: 337 - DNA, 21 bp, Artificial Sequence
  siRNA selective for SHIP1
ccctctctctctttctctctc

SEQ ID NO: 338 - DNA, 21 bp, Artificial Sequence
  siRNA selective for SHIP1
gggggagagagagaaagagag

SEQ ID NO: 339 - DNA, 21 bp, Artificial Sequence
  siRNA selective for SHIP1
ggccgaggaggcccacgccca

SEQ ID NO: 340 - DNA, 21 bp, Artificial Sequence
  siRNA selective for SHIP1
ggccggctcctccgggtgcgg

SEQ ID NO: 341 - DNA, 21 bp, Artificial Sequence
  siRNA selective for SHIP1
gccgaggaggcccacgcccac

SEQ ID NO: 342 - DNA, 21 bp, Artificial Sequence
  siRNA selective for SHIP1
gccggctcctccgggtgcggg

SEQ ID NO: 343 - DNA, 21 bp, Artificial Sequence
  siRNA selective for SHIP1
tctttctctctctctctcttg

SEQ ID NO: 344 - DNA, 21 bp, Artificial Sequence
  siRNA selective for SHIP1
agagaaagagagagagagaga

SEQ ID NO: 345 - DNA, 21 bp, Artificial Sequence
  siRNA selective for SHIP1
cctctctctctttctctctct

SEQ ID NO: 346 - DNA, 21 bp, Artificial Sequence
  siRNA selective for SHIP1
ggggagagagagaaagagaga

SEQ ID NO: 347 - DNA, 21 bp, Artificial Sequence
  siRNA selective for SHIP1
ctctctctctttctctctctc

SEQ ID NO: 348 - DNA, 21 bp, Artificial Sequence
  siRNA selective for SHIP1
gggagagagagaaagagagag

SEQ ID NO: 349 - DNA, 21 bp, Artificial Sequence
  siRNA selective for SHIP1
ctctttctctctctctctctt

SEQ ID NO: 350 - DNA, 21 bp, Artificial Sequence
  siRNA selective for SHIP1
gagagaaagagagagagagag

SEQ ID NO: 351 - DNA, 21 bp, Artificial Sequence
  siRNA selective for SHIP1
tctctctctttctctctctct

SEQ ID NO: 352 - DNA, 21 bp, Artificial Sequence
  siRNA selective for SHIP1
ggagagagagaaagagagaga

SEQ ID NO: 353 - DNA, 21 bp, Artificial Sequence
  siRNA selective for SHIP1
ctctctctttctctctctctc

SEQ ID NO: 354 - DNA, 21 bp, Artificial Sequence
  siRNA selective for SHIP1
gagagagagaaagagagagag

SEQ ID NO: 355 - DNA, 21 bp, Artificial Sequence
  siRNA selective for SHIP1
tctctctttctctctctctct

SEQ ID NO: 356 - DNA, 21 bp, Artificial Sequence
  siRNA selective for SHIP1
agagagagaaagagagagaga

SEQ ID NO: 357 - DNA, 21 bp, Artificial Sequence
  siRNA selective for SHIP1
ctctctttctctctctctctc

SEQ ID NO: 358 - DNA, 21 bp, Artificial Sequence
  siRNA selective for SHIP1
gagagagaaagagagagagag

SEQ ID NO: 359 - DNA, 21 bp, Artificial Sequence
  siRNA selective for SHIP1
tctctttctctctctctctct

SEQ ID NO: 360 - DNA, 21 bp, Artificial Sequence
  siRNA selective for SHIP1
agagagaaagagagagagaga

SEQ ID NO: 361 - DNA, 19 bp, Artificial Sequence
  siRNA selective for SHIP1
cccatatcacccaagaagt

SEQ ID NO: 362 - DNA, 97 bp, Artificial Sequence
  shRNA selective for SHIP1
tgctgttgacagtgagcgagcccatatcacccaagaagtttagtgaagccacagatgtaaacttcttgggtgatatgggcgtgcctactgcctcgga

SEQ ID NO: 363 - DNA, 19 bp, Artificial Sequence
  siRNA selective for SHIP1
gcttccagaagagcatctt

SEQ ID NO: 364 - DNA, 97 bp, Artificial Sequence
  shRNA selective for SHIP1
tgctgttgacagtgagcgaggcttccagaagagcatcttatagtgaagccacagatgtataagatgctcttctggaagccctgcctactgcctcgga

SEQ ID NO: 365 - DNA, 19 bp, Artificial Sequence
  siRNA selective for SHIP1
catatcctgatcagcatta

SEQ ID NO: 366 - DNA, 97 bp, Artificial Sequence
  shRNA selective for SHIP1
tgctgttgacagtgagcgcgcatatcctgatcagcattaatagtgaagccacagatgtattaatgctgatcaggatatgcttgcctactgcctcgga

SEQ ID NO: 367 - DNA, 19 bp, Artificial Sequence
  siRNA selective for SHIP1
ctgtatcggaattgcgttt

SEQ ID NO: 368 - DNA, 97 bp, Artificial Sequence
  shRNA selective for SHIP1
tgctgttgacagtgagcgcgctgtatcggaattgcgtttatagtgaagccacagatgtataaacgcaattccgatacagcatgcctactgcctcgga

SEQ ID NO: 369 - DNA, 19 bp, Artificial Sequence
  siRNA selective for SHIP1
cttatgaggatggaaggaa

SEQ ID NO: 370 - DNA, 97 bp, Artificial Sequence
  shRNA selective for SHIP1
tgctgttgacagtgagcgcgcttatgaggatggaaggaattagtgaagccacagatgtaattccttccatcctcataagcttgcctactgcctcgga

SEQ ID NO: 371 - DNA, 19 bp, Artificial Sequence
  siRNA selective for SHIP1
ctgctttccaggacaggca

SEQ ID NO: 372 - DNA, 97 bp, Artificial Sequence
  shRNA selective for SHIP1
tgctgttgacagtgagcgcgctgctttccaggacaggcaatagtgaagccacagatgtattgcctgtcctggaaagcagcttgcctactgcctcgga

SEQ ID NO: 373 - DNA, 19 bp, Artificial Sequence
  siRNA selective for SHIP1
ctgaaagccatccaggatt

SEQ ID NO: 374 - DNA, 97 bp, Artificial Sequence
  shRNA selective for SHIP1
tgctgttgacagtgagcgccctgaaagccatccaggattatagtgaagccacagatgtataatcctggatggctttcaggttgcctactgcctcgga

SEQ ID NO: 375 - DNA, 19 bp, Artificial Sequence
  siRNA selective for SHIP1
ccgcccatatcacccaaga

SEQ ID NO: 376 - DNA, 97 bp, Artificial Sequence
  shRNA selective for SHIP1
tgctgttgacagtgagcgcgccgcccatatcacccaagaatagtgaagccacagatgtattcttgggtgatatgggcggcttgcctactgcctcgga

SEQ ID NO: 377 - DNA, 19 bp, Artificial Sequence
  siRNA selective for SHIP1
gtgcgtgccagcgagtcca

SEQ ID NO: 378 - DNA, 97 bp, Artificial Sequence
  shRNA selective for SHIP1
tgctgttgacagtgagcgccgtgcgtgccagcgagtccattagtgaagccacagatgtaatggactcgctggcacgcacgatgcctactgcctcgga

SEQ ID NO: 379 - DNA, 19 bp, Artificial Sequence
  siRNA selective for SHIP1
ctctgcgtgctgtatcgga

SEQ ID NO: 380 - DNA, 97 bp, Artificial Sequence
  shRNA selective for SHIP1
tgctgttgacagtgagcgagctctgcgtgctgtatcggaatagtgaagccacagatgtattccgatacagcacgcagagcgtgcctactgcctcgga

SEQ ID NO: 381 - DNA, 19 bp, Artificial Sequence
  siRNA selective for SHIP1
ctcattaagtcacagaaat

SEQ ID NO: 382 - DNA, 97 bp, Artificial Sequence
  shRNA selective for SHIP1
tgctgttgacagtgagcgcgctcattaagtcacagaaatttagtgaagccacagatgtaaatttctgtgacttaatgagcttgcctactgcctcgga

SEQ ID NO: 383 - DNA, 19 bp, Artificial Sequence
  siRNA selective for SHIP1
cgagtcctctggaagtctt

SEQ ID NO: 384 - DNA, 97 bp, Artificial Sequence
  shRNA selective for SHIP1
tgctgttgacagtgagcgcccgagtcctctggaagtcttatagtgaagccacagatgtataagacttccagaggactcggttgcctactgcctcgga

SEQ ID NO: 385 - DNA, 19 bp, Artificial Sequence
  siRNA selective for SHIP1
gagtccatctcccgggcat

SEQ ID NO: 386 - DNA, 97 bp, Artificial Sequence
  shRNA selective for SHIP1
tgctgttgacagtgagcgacgagtccatctcccgggcatatagtgaagccacagatgtatatgcccgggagatggactcgctgcctactgcctcgga

SEQ ID NO: 387 - DNA, 19 bp, Artificial Sequence
  siRNA selective for SHIP1
gagagactcttcccaagct

SEQ ID NO: 388 - DNA, 97 bp, Artificial Sequence
  shRNA selective for SHIP1
tgctgttgacagtgagcgcggagagactcttcccaagctatagtgaagccacagatgtatagcttgggaagagtctctccatgcctactgcctcgga

SEQ ID NO: 389 - DNA, 19 bp, Artificial Sequence
  siRNA selective for SHIP1
cgggatgaatccagtggaa

SEQ ID NO: 390 - DNA, 97 bp, Artificial Sequence
  shRNA selective for SHIP1
tgctgttgacagtgagcgcgcgggatgaatccagtggaattagtgaagccacagatgtaattccactggattcatcccgcttgcctactgcctcgga

SEQ ID NO: 391 - DNA, 19 bp, Artificial Sequence
  siRNA selective for SHIP1
ccgagcctctccgagacat

SEQ ID NO: 392 - DNA, 97 bp, Artificial Sequence
  shRNA selective for SHIP1
tgctgttgacagtgagcgagccgagcctctccgagacatttagtgaagccacagatgtaaatgtctcggagaggctcggcctgcctactgcctcgga

SEQ ID NO: 393 - DNA, 19 bp, Artificial Sequence
  siRNA selective for SHIP1
cccaaacccaccagtttaa

SEQ ID NO: 394 - DNA, 97 bp, Artificial Sequence
  shRNA selective for SHIP1
tgctgttgacagtgagcgcgcccaaacccaccagtttaaatagtgaagccacagatgtatttaaactggtgggtttgggcatgcctactgcctcgga

SEQ ID NO: 395 - DNA, 19 bp, Artificial Sequence
  siRNA selective for SHIP1
gctggtgacccatctgcaa

SEQ ID NO: 396 - DNA, 97 bp, Artificial Sequence
  shRNA selective for SHIP1
tgctgttgacagtgagcgaggctggtgacccatctgcaattagtgaagccacagatgtaattgcagatgggtcaccagccctgcctactgcctcgga

SEQ ID NO: 397 - DNA, 19 bp, Artificial Sequence
  siRNA selective for SHIP1
ctgacgaagcccgagatgt

SEQ ID NO: 398 - DNA, 97 bp, Artificial Sequence
  shRNA selective for SHIP1
tgctgttgacagtgagcgagctgacgaagcccgagatgtttagtgaagccacagatgtaaacatctcgggcttcgtcagcgtgcctactgcctcgga

SEQ ID NO: 399 - DNA, 19 bp, Artificial Sequence
  siRNA selective for SHIP1
cccatatcacccaagaagt

SEQ ID NO: 400 - DNA, 97 bp, Artificial Sequence; identical to SEQ ID NO: 362
  shRNA selective for SHIP1

SEQ ID NO: 401 - DNA, 19 bp, Artificial Sequence
  siRNA selective for SHIP1
gcttccagaagagcatctt

SEQ ID NO: 402 - DNA, 97 bp, Artificial Sequence; identical to SEQ ID NO: 364
  shRNA selective for SHIP1

SEQ ID NO: 403 - DNA, 19 bp, Artificial Sequence
  siRNA selective for SHIP1
ctgtatcggaattgcgttt

SEQ ID NO: 404 - DNA, 97 bp, Artificial Sequence; identical to SEQ ID NO: 368
  shRNA selective for SHIP1

SEQ ID NO: 405 - DNA, 19 bp, Artificial Sequence
  siRNA selective for SHIP1
cttatgaggatggaaggaa

SEQ ID NO: 406 - DNA, 97 bp, Artificial Sequence; identical to SEQ ID NO: 370
  shRNA selective for SHIP1

SEQ ID NO: 407 - DNA, 19 bp, Artificial Sequence
  siRNA selective for SHIP1
ctgctttccaggacaggca

SEQ ID NO: 408 - DNA, 97 bp, Artificial Sequence; identical to SEQ ID NO: 372
  shRNA selective for SHIP1

SEQ ID NO: 409 - DNA, 19 bp, Artificial Sequence
  siRNA selective for SHIP1
ccgcccatatcacccaaga

SEQ ID NO: 410 - DNA, 97 bp, Artificial Sequence; identical to SEQ ID NO: 376
  shRNA selective for SHIP1

SEQ ID NO: 411 - DNA, 19 bp, Artificial Sequence
  siRNA selective for SHIP1
gtgcgtgccagcgagtcca

SEQ ID NO: 412 - DNA, 97 bp, Artificial Sequence; identical to SEQ ID NO: 378
  shRNA selective for SHIP1

SEQ ID NO: 413 - DNA, 19 bp, Artificial Sequence
  siRNA selective for SHIP1
ctctgcgtgctgtatcgga

SEQ ID NO: 414 - DNA, 97 bp, Artificial Sequence; identical to SEQ ID NO: 380
  shRNA selective for SHIP1

SEQ ID NO: 415 - DNA, 19 bp, Artificial Sequence
  siRNA selective for SHIP1
ctcattaagtcacagaaat

SEQ ID NO: 416 - DNA, 97 bp, Artificial Sequence; identical to SEQ ID NO: 382
  shRNA selective for SHIP1

SEQ ID NO: 417 - DNA, 19 bp, Artificial Sequence
  siRNA selective for SHIP1
cgagtcctctggaagtctt

SEQ ID NO: 418 - DNA, 97 bp, Artificial Sequence; identical to SEQ ID NO: 384
  shRNA selective for SHIP1

SEQ ID NO: 419 - DNA, 19 bp, Artificial Sequence
  siRNA selective for SHIP1
gagtccatctcccgggcat

SEQ ID NO: 420 - DNA, 97 bp, Artificial Sequence; identical to SEQ ID NO: 386
  shRNA selective for SHIP1

SEQ ID NO: 421 - DNA, 19 bp, Artificial Sequence
  siRNA selective for SHIP1
ccgagcctctccgagacat

SEQ ID NO: 422 - DNA, 97 bp, Artificial Sequence; identical to SEQ ID NO: 392
  shRNA selective for SHIP1

SEQ ID NO: 423 - DNA, 19 bp, Artificial Sequence
  siRNA selective for SHIP1
cccaaacccaccagtttaa

SEQ ID NO: 424 - DNA, 97 bp, Artificial Sequence; identical to SEQ ID NO: 394
  shRNA selective for SHIP1

SEQ ID NO: 425 - DNA, 19 bp, Artificial Sequence
  siRNA selective for SHIP1
gctggtgacccatctgcaa

SEQ ID NO: 426 - DNA, 97 bp, Artificial Sequence; identical to SEQ ID NO: 396
  shRNA selective for SHIP1

SEQ ID NO: 427 - DNA, 19 bp, Artificial Sequence
  siRNA selective for SHIP1
ctgacgaagcccgagatgt

SEQ ID NO: 428 - DNA, 97 bp, Artificial Sequence; identical to SEQ ID NO: 398
  shRNA selective for SHIP1